(19) United States
(12) Patent Application Publication
(10) Pub. No.: US 2023/0371363 A1
(43) Pub. Date: Nov. 16, 2023

(54) COMPOUNDS THAT CAN BE USED FOR STRUCTURING FUNCTIONAL LAYERS OF ORGANIC ELECTROLUMINESCENT DEVICES

(71) Applicant: Merck Patent GmbH, Darmstadt (DE)

(72) Inventor: Philipp STOESSEL, Darmstadt (DE)

(21) Appl. No.: 18/029,114

(22) PCT Filed: Sep. 28, 2021
(86) PCT No.: PCT/EP2021/076552

§ 371 Date: Mar. 29, 2023

(30) Foreign Application Priority Data
Sep. 30, 2020 (EP) 20199158.5

(51) Int. Cl. H10K 85/60; C07C 22/04; C07D 235/04; C07D 401/14; C07D 251/24; C07D 403/04; C07F 5/02

(52) U.S. Cl. CPC H10K 85/615; H10K 85/654; C07C 22/04; C07D 235/04; C07D 401/14; C07D 251/24; C07D 403/04; C07F 5/027

(57) ABSTRACT

The present invention relates to the use of compounds for structuring of at least one functional layer of an organic electronic device. The present invention further relates to preferred compounds suitable for use in electronic devices, and to electronic devices, especially organic electroluminescent devices, comprising these compounds.

The present invention relates to compounds for use in electronic devices, especially in organic electroluminescent devices, and to electronic devices, especially organic electroluminescent devices comprising these compounds.

Organic electronic devices, for example organic electroluminescent devices, generally comprise multiple layers of organic materials disposed between conductive thin-film electrodes. If a voltage is applied to electrodes, holes and electrons are respectively injected by an anode and a cathode. Holes and electrons can then combine to give a bound state, which is referred to as an exciton. Excitons can decay especially in an emitting layer with emission of photons.

In more recent devices, attempts have been made to configure the respective layers with minimum width in order to improve the transparency of the layers or other desirable properties, for example quantum efficiency. However, a reduction in the thickness of a layer is accompanied by an increase in its sheet resistance.

An electrode having a high sheet resistance is generally undesirable for use in organic electroluminescent devices, since it creates a large drop in current resistance (IR) when a device is used, which has an adverse effect on the performance and efficiency of organic electroluminescent devices. The IR drop can be compensated for to a certain degree by increasing the power supply level. But if the power supply level for a pixel is increased, the voltages delivered to other components are also increased in order to maintain proper operation of the device, and are therefore unfavourable.

In order to reduce the power supply specifications for OLED devices with the highest emission, the formation of collector guide structures or auxiliary electrodes on the devices has been proposed as solutions.

For example, such an auxiliary electrode can be implemented by depositing a conductive coating connected to an electrode in an electrically conductive manner. Such an auxiliary electrode can serve to guide current more effectively to various regions of the device, such that sheet resistance and any associated IR drop of the electrode is reduced.

Since an auxiliary electrode is typically provided on an OLED stack containing an anode, one or more organic layers and a cathode, the structuring of the auxiliary electrode is traditionally achieved using a shadow mask with mask openings, through which a conductive coating is selectively deposited, for example by physical vapour deposition (PVD) methods. However, this method is very prone to error, it being necessary to clean the masks used in a complex manner or dispose of them after use. Therefore, such processes are not in commercial use.

A further procedure for structuring is set out in WO 2019/150327 A1; according to this application, relatively few compounds are effectively suitable for prevention of metal deposition, and these compounds have a very complex structure. Furthermore, the publication does not provide any clear teaching about appropriate compounds since some structurally very similar compounds show very different results.

There is generally still a need for improvement in electroluminescent devices.

There is generally still a need for improvement in these compounds that can especially be used for the structuring of functional layers. For instance, these compounds should especially be usable for production of improved auxiliary electrodes or similar structures. At the same time, other properties of the organic electronic devices, especially the lifetime thereof and colour purity thereof, but also the efficiency thereof and operating voltage thereof, should not be adversely affected.

It is therefore an object of the present invention to provide compounds which are suitable for use in an organic electronic device, especially in an organic electroluminescent device, and which lead to good device properties when used in this device, and to provide the corresponding electronic device.

More particularly, it is an object of the present invention to provide compounds with which layers of electronic devices can be structured easily, reliably and inexpensively. At the same time, it should especially be possible to create structures that reduce the resistance of the electronic device or the layers thereof with the aid of the present compounds.

It is a further object of the present invention to provide compounds which lead to a high lifetime, good efficiency and low operating voltage.

In addition, the compounds should have excellent processibility, and the compounds should especially show good solubility.

A further object of the present invention can be considered that of providing compounds suitable for use in phosphorescent or fluorescent electroluminescent devices, especially in an antideposition layer.

A further object can be considered that of providing electronic devices having excellent performance as inexpensively as possible and in constant quality.

Furthermore, it should be possible to use or adapt the electronic devices for many purposes. More particularly, the performance of the electronic devices should be maintained over a broad temperature range.

It has been found that, surprisingly, this object is achieved by particular compounds described in detail below that are of very good suitability for use in organic electronic devices, preferably electroluminescent devices, and lead to organic electronic devices, preferably organic electroluminescent devices, that show very good properties, especially in relation to lifetime, colour purity, efficiency and operating voltage. The present invention therefore provides these compounds and electronic devices, especially organic electroluminescent devices, comprising such compounds.

The present invention therefore provides for use of a compound for structuring of at least one functional layer of an organic electronic device, wherein the compound comprises at least one fluorinated alkyl radical having at least two carbon atoms.

Functional layers of an electronic device are known to the person skilled in the art, these being described above and hereinafter, and so reference is made thereto.

The term “structuring” herein refers to the creation of a structure in or on a functional layer. These structures may serve here, for example, for creation of electrically conductive units, especially of auxiliary electrodes, that result in a reduction in the resistance of the electronic device and/or in the operating voltage, this being described above and hereinafter, and so reference is made thereto.

In a preferred embodiment, it may be the case that the fluorinated alkyl radical comprises at least 2 and more preferably at least 3 fluorine atoms.

The fluorinated alkyl radical preferably comprises not more than 20, preferably not more than 16, more preferably not more than 12 and especially preferably not more than 10 carbon atoms.

It may further be the case that the fluorinated alkyl radical has a numerical ratio of fluorine atoms to carbon atoms of at least 0.5, preferably of at least 0.75 and more preferably of at least 1.

In a further configuration, it may be the case that the fluorinated alkyl radical has a numerical ratio of hydrogen atoms to fluorine atoms of not more than 1, preferably not more than 0.75 and more preferably not more than 0.5, where the fluorinated alkyl radical more preferably comprises not more than 10, preferably not more than 6, more preferably not more than 4 and especially preferably no hydrogen atoms.

It may further be the case that the fluorinated alkyl radical comprises preferably 2 to 20, more preferably 3 to 10, carbon atoms.

It may additionally be the case that the fluorinated alkyl radical comprises or is a cyclic group, where preferably some of the carbon atoms have bonds to at least two hydrogen atoms and some of the carbon atoms have bonds to at least two fluorine atoms. The fluorinated alkyl radical is preferably linear or branched, more preferably linear, where preferably some of the carbon atoms have bonds to at least two hydrogen atoms and some of the carbon atoms have bonds to at least two fluorine atoms.

In a preferred configuration, it may be the case that the fluorinated alkyl radical has a block structure, where some of the carbon atoms have bonds to at least two hydrogen atoms and some of the carbon atoms have bonds to at least two fluorine atoms. The term “block structure” is known in the specialist field and includes the possibility of the fluorinated alkyl radical having block structures, where a single CH₂, CHF or CF₂ group may also be considered as a block.

In a preferred embodiment, it may be the case that the fluorinated alkyl radical has a structure of the formulae FA-1 to FA-16

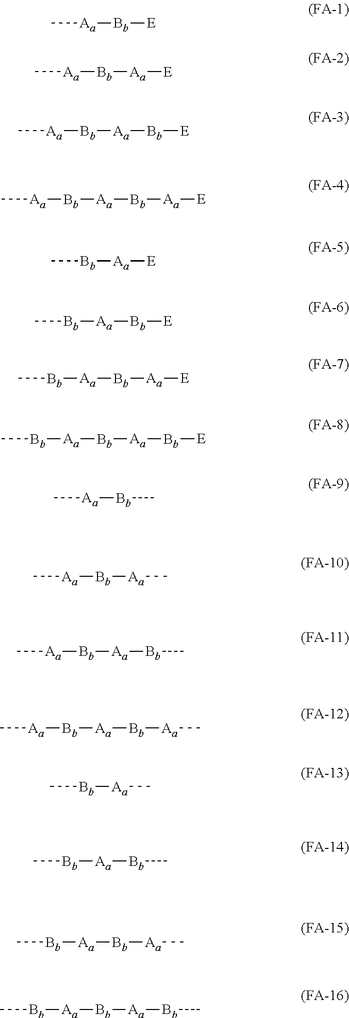

where the dotted line represents the site of attachment of the fluorinated alkyl radical, and in addition:
A is a group of the formula —(CₓH₂ₓ)—, —(CₓHₓDₓ)—, —(CₓD₂ₓ)—, where x is an integer in the range from 1 to 6, preferably 1 to 4, more preferably one or two, where A is more preferably selected from —(CH₂)—, —(CHD)— or —(CD₂)—, —(CH₂CH₂)—, —(CHD—CHD)— or —(CD₂CD₂)— and is especially preferably —(CH₂)— or —(CH₂CH₂)—;
B is a group of the formula —(C_{y}F_{2y})—, —(C_{y}F_{y}H_{y})—, —(C_{y}F_{y}D_{y})—, where y is an integer in the range from 1 to 6, preferably 1 to 4, more preferably 1, 2 or 3, where B is more preferably selected from —(CF₂CF₂CF₂)—, —(CFH—CFH—CFH)—, —(CFD—CFD—CFD)—, —(CF₂CF₂)—, —(CFH—CFH)—, —(CFD—CFD)—, —(CF₂)—, —(CFH)— or —(CFD)—, and is especially preferably —(CF₂CF₂CF₂)—, —(CF₂CF₂)—, or —(CF₂)—;
E is selected from H, D or F, preferably F;
a is an integer in the range from 1 to 6, preferably 1 to 4, more preferably 1 or 2;
b is an integer in the range from 1 to 6, preferably 1 to 4, more preferably 1 or 2;
   where the structures of the formulae (FA-9) to (FA-16) may form a ring, but are preferably linear or branched, and are more preferably linear, and are connected at two sites to further groups in the compound, preference being given to the structures of the formulae (FA-1) to (FA-8), and particular preference to the structures of the formulae (FA-1) to (FA-4).

The present invention preferably provides for the use of a compound comprising at least one structuring element of the formula (SE-I), (SE-II) and/or (SE-III)

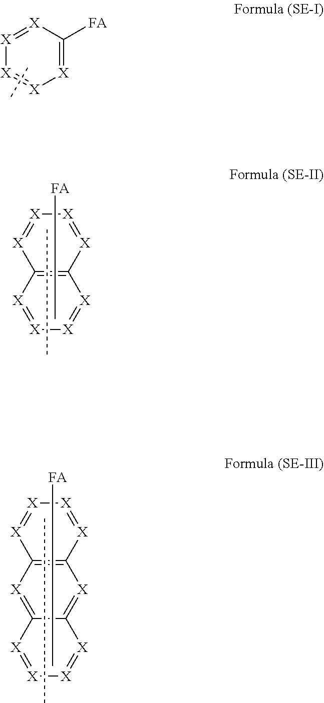

where the FA group is a fluorinated alkyl radical which has at least two carbon atoms and may be substituted by one or more R radicals, but is preferably unsubstituted, the dotted bonds represent the bonding site, and in addition:
X is CR, N or C if a group binds to X, preferably CR or C;
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹,
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more preferably adjacent R¹ radicals together may form a ring system; at the same time, one or more R¹ radicals may form a ring system with a further part of the compound;
Ar″ is the same or different at each instance and is an aromatic or heteroaromatic ring system which has 5 to 30 aromatic ring atoms and may be substituted by one or more R² radicals; at the same time, it is possible for two Ar″ radicals bonded to the same carbon atom, silicon atom, nitrogen atom, phosphorus atom or boron atom also to be joined together via a bridge by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, preferably adjacent substituents R² together may form a ring system.

Preference is given here to structures of the formula (SE-I) and (SE-II), and particular preference to structures of the formula (SE-I).

It may further be the case that the structuring element of formula (SE-I), (SE-II) and/or (SE-III) has exactly 1, 2, 3 or 4 FA groups, where one or more of the FA groups is optionally given by one or more of the substituents R.

It may preferably be the case that the structuring element of formula (SE-I), (SE-II) and/or (SE-III) has at least 1, preferably at least 2 and more preferably at least 3 fluorine atoms.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. Here, an aryl group or heteroaryl group is understood to mean either a simple aromatic ring, i.e. benzene, or a simple heteroaromatic ring, for example pyridine, pyrimidine, thiophene, etc., or a condensed (fused) aryl or heteroaryl group, for example naphthalene, anthracene, phenanthrene, quinoline, isoquinoline, etc. Aromatic systems joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, further preferably F or CN, especially preferably CN.

An aromatic or heteroaromatic ring system which has 5 to 60 or 5 to 40 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

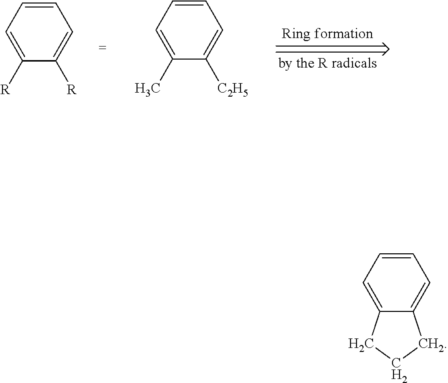

In addition, however, the abovementioned wording shall also be understood to mean that, if one of the two radicals is hydrogen, the second radical binds to the position to which the hydrogen atom was bonded, forming a ring. This shall be illustrated by the following scheme:

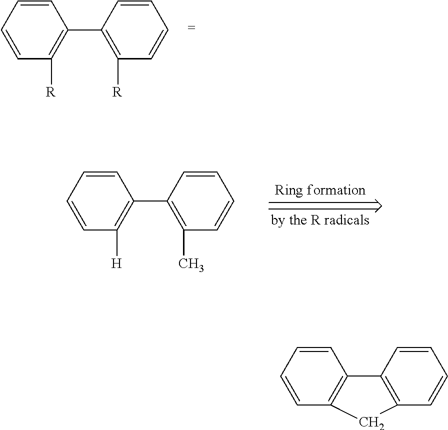

It may preferably be the case that the FA group in a structuring element of formula (SE-I), (SE-II) and/or (SE-III) has at least 1, preferably at least 2 and more preferably at least 3 fluorine atoms. It may further be the case that at least one X group, preferably at least two of the X groups, in a structuring element of formula (SE-I), (SE-II) and/or (SE-III) comprises at least 1, preferably at least 2 and more preferably at least 3 fluorine atoms, where more preferably at least one X group, preferably at least two of the X groups, represent(s) a radical of the formula CF.

In a further preferred configuration, it may be the case that the FA group of the structuring element of formula (SE-I), (SE-II) and/or (SE-III) comprises, preferably corresponds to, at least one of the structures of the formulae (FA-1) to (FA-16) shown above.

In a preferred configuration of the present invention, it may be the case that the structuring element of the formula (SE-I), (SE-II) and/or (SE-III) can be represented by a formula (SE-I) to (SE-21)

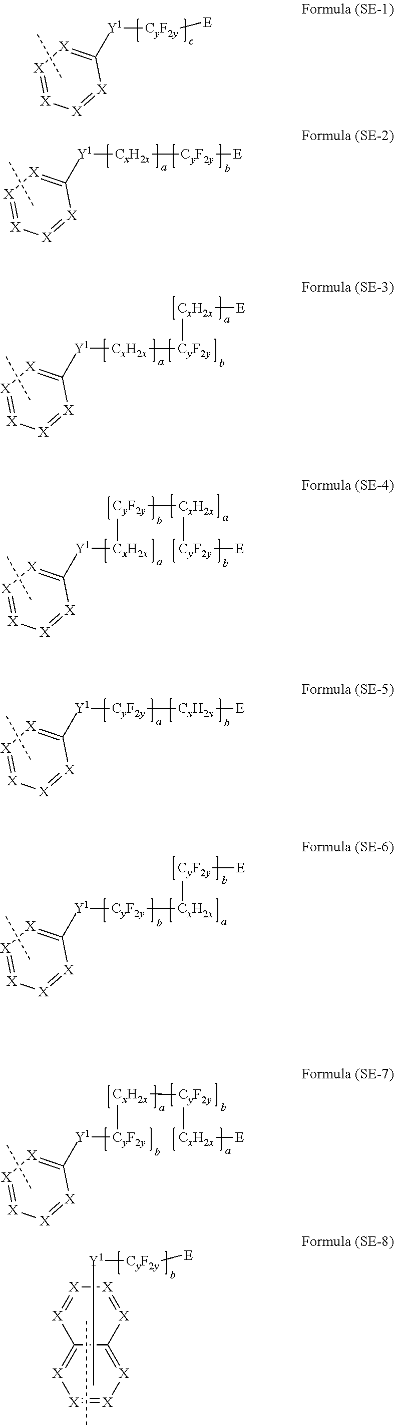

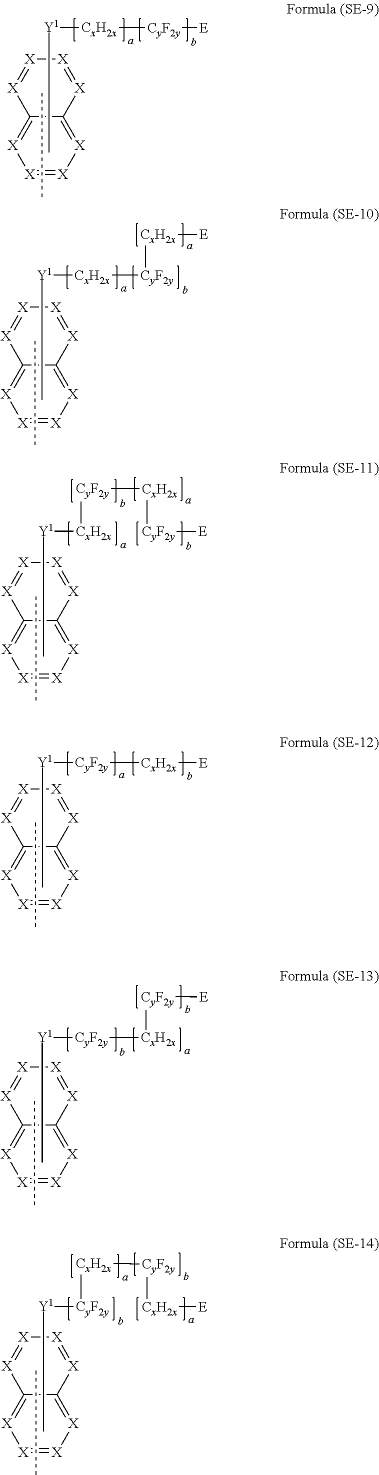

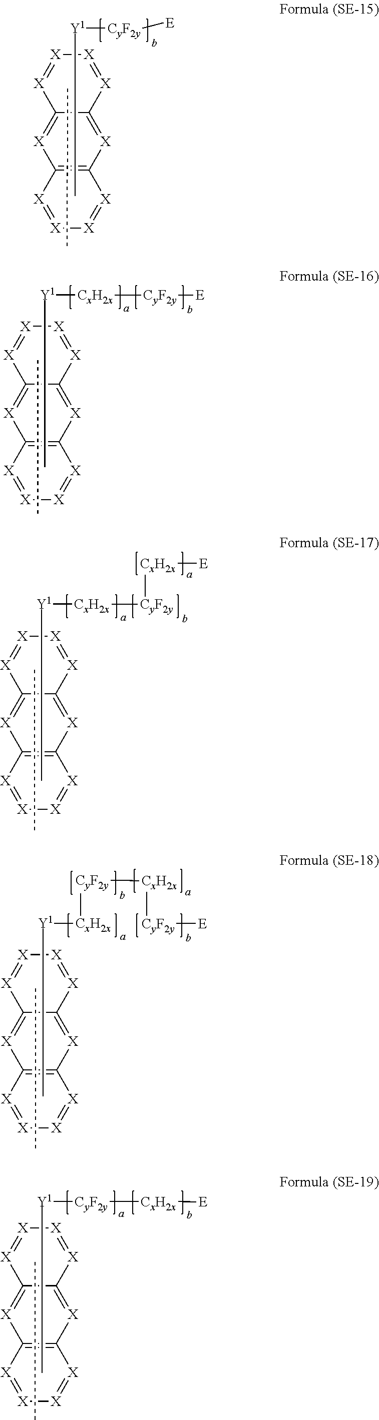

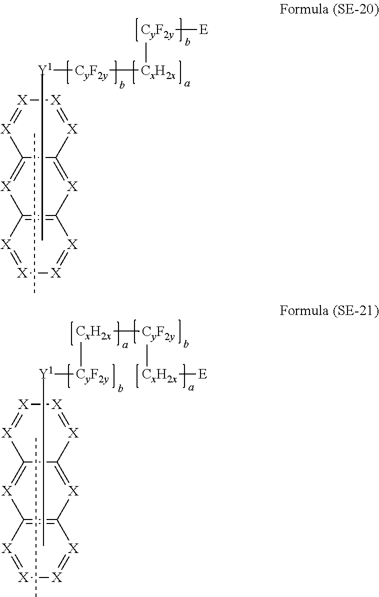

where the symbol X has the definition given above, especially for formula (SE-I), (SE-II) and/or (SE-III), the dotted bonds means attachment site, and in addition:
E is selected from H, D or F, preferably H or F;
Y¹ is the same or different at each instance and is a bond, O, S, NR³ or C(═O), preferably a bond, O, S, NR³, more preferably a bond, O or S, especially preferably a bond;
R³ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, the R³ radical may form a ring system together with an adjacent R or R¹ radical, where the R² and Ar″ radicals have the definitions given above, especially for formula (SE-I), (SE-II) and/or (SE-III),
the index a is the same or different at each instance and is 1, 2, 3, 4, 5 or 6, preferably 1, 2, 3 or 4, more preferably 1 or 2;
the index b is the same or different at each instance and is 1, 2, 3, 4, 5 or 6, preferably 1, 2, 3 or 4, more preferably 1 or 2;
the index c is 2, 3, 4, 5, 6, 7, 8, 9 or 10, preferably 2, 3, 4, 5 or 6, more preferably 2, 3 or 4, most preferably 2 or 3;
the index x is the same or different at each instance and is 1, 2, 3, 4, 5 or 6, preferably 1, 2, 3 or 4, more preferably 1 or 2;
the index y is the same or different at each instance and is 1, 2, 3, 4, 5 or 6, preferably 1, 2, 3 or 4, more preferably 1 or 2.

Preferably, in formula (SE-I), (SE-II), (SE-III) and/or (SE-I) to (SE-21), not more than two, preferably not more than one, of the X group(s) is N; more preferably, all X groups are CR or C.

It may preferably further be the case that the structuring element of the formula (SE-I), (SE-II) and/or (SE-III) can be represented by a formula (SE-1a) to (SE-21a)

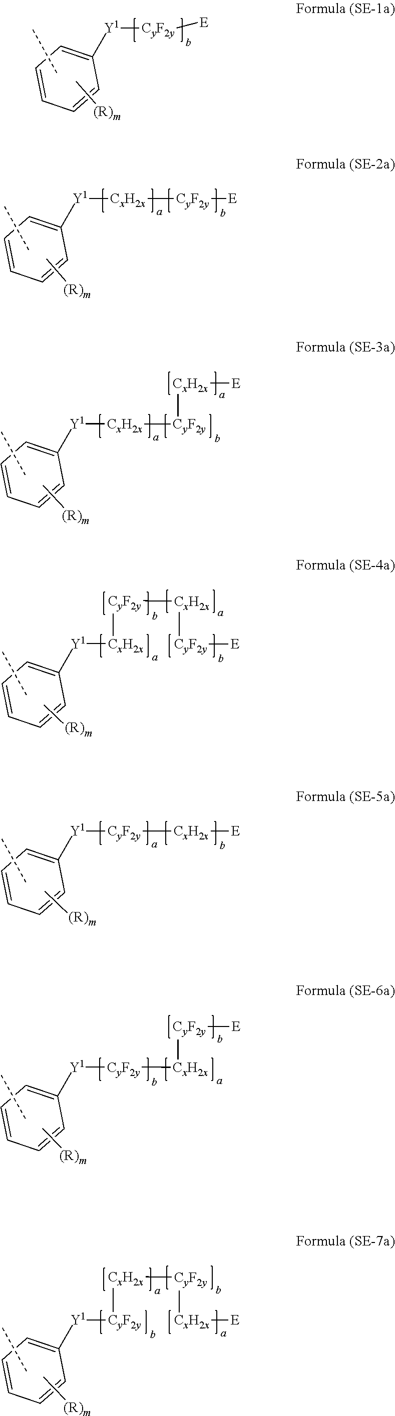

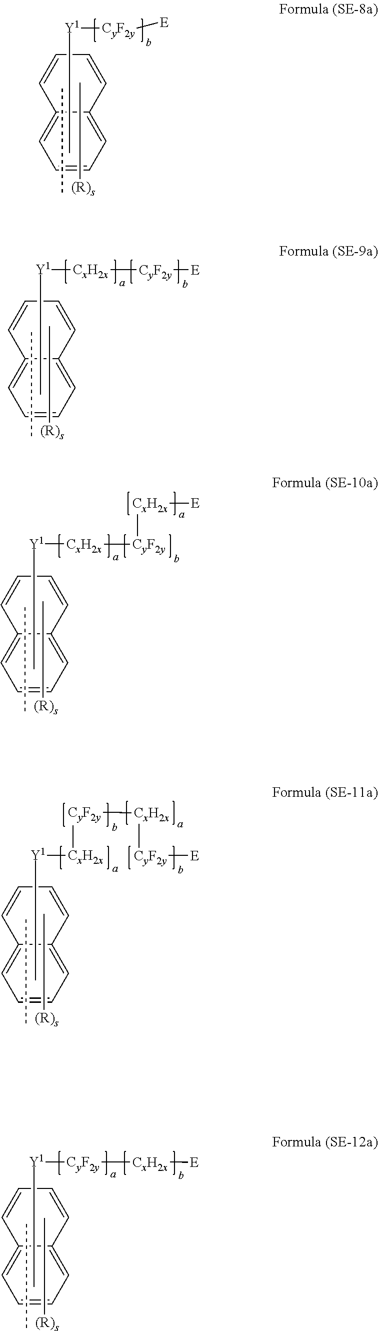

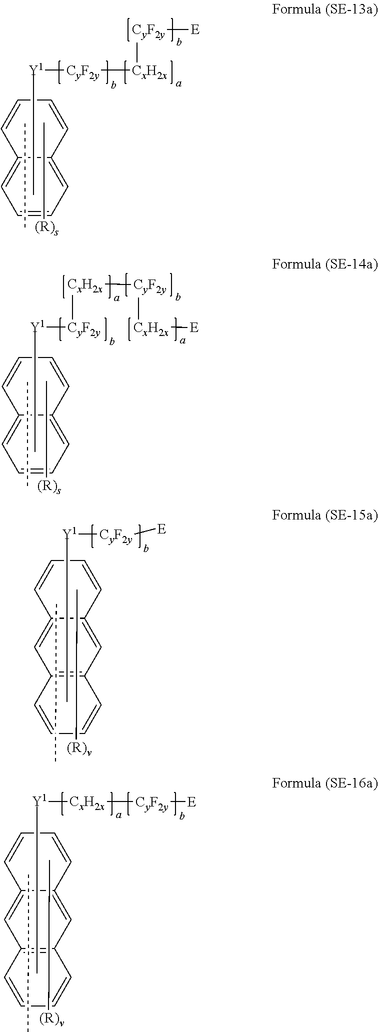

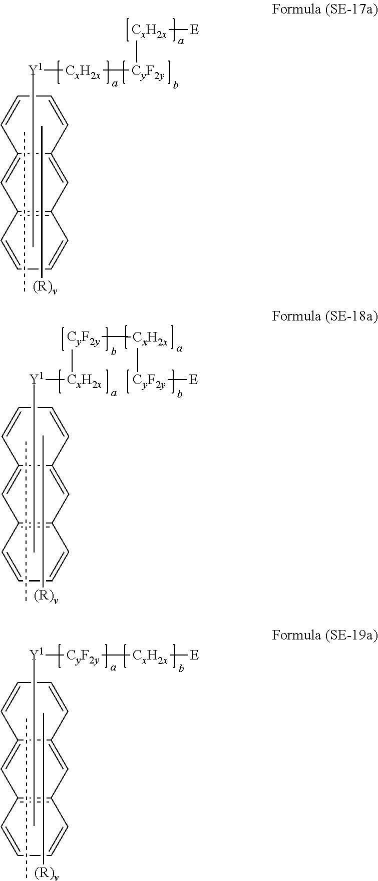

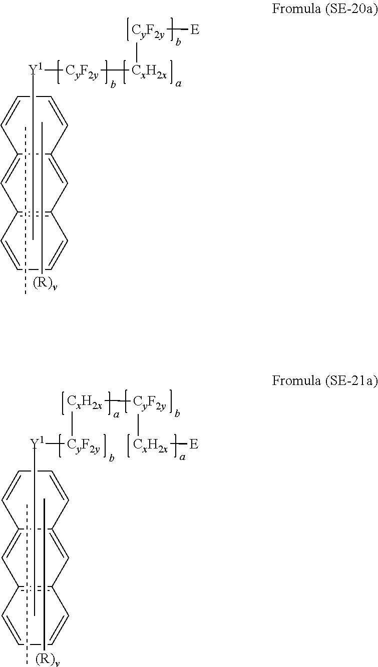

where the symbol R has the definition given above, especially for formula (SE-I), (SE-II) and/or (SE-III), the symbols Y¹ and E and the indices a, b, c, x and y have the definitions given above, especially for formulae (SE-I) to (SE-21), the dotted bond indicates attachment site, and the further symbols are as follows:
m is 0, 1, 2, 3 or 4, preferably 0, 1 or 2, more preferably 0 or 1;
s is 0, 1, 2, 3, 4, 5 or 6, preferably 0, 1, 2, 3 or 4, more preferably 0, 1 or 2;
v is 0, 1, 2, 3, 4, 5, 6, 7 or 8, preferably 0, 1, 2, 3 or 4, more preferably 0, 1 or 2.

Preferably, two R radicals together with the heteroaromatic or aromatic groups to which these R radicals bind do not form a fused aromatic or heteroaromatic ring system, this including possible substituents R¹, R² by which the R radicals may be substituted. This is especially true of the structures of the formulae (SE-I), (SE-II), (SE-III), (SE-I) to (SE-21) and (SE-1a) to (SE-21a) and the further preferred configurations of these structures and compounds that are described above and hereinafter.

It may preferably be the case that the sum total of the indices a and x is not more than 10, preferably not more than 7 and more preferably not more than 5, and that of the indices b and y is not more than 10, preferably not more than 7 and more preferably not more than 5. This preference is applicable especially to structures of the formulae (SE-1a) to (SE-21a) and the further preferred configurations of these structures and compounds that are described above and hereinafter.

In a further preferred configuration, it may be the case that at least two, preferably at least three, of the R and/or R¹ radicals are F or a fluorinated alkyl radical having 1 to 20 carbon atoms. This preference is applicable especially to structures of the formulae (SE-I), (SE-II), (SE-III), (SE-I) to (SE-21) and (SE-1a) to (SE-21a) and the further preferred configurations of these structures and compounds that are described above and hereinafter.

A compound usable with preference for the use according to the invention preferably comprises at least one aromatic or heteroaromatic ring system having at least two, preferably having at least three, fused aromatic or heteroaromatic rings.

In a preferred configuration, it may the case that the aromatic or heteroaromatic ring system having two, preferably having three, fused aromatic or heteroaromatic rings is selected from the groups of the formulae (Ar-1) to (Ar-18)

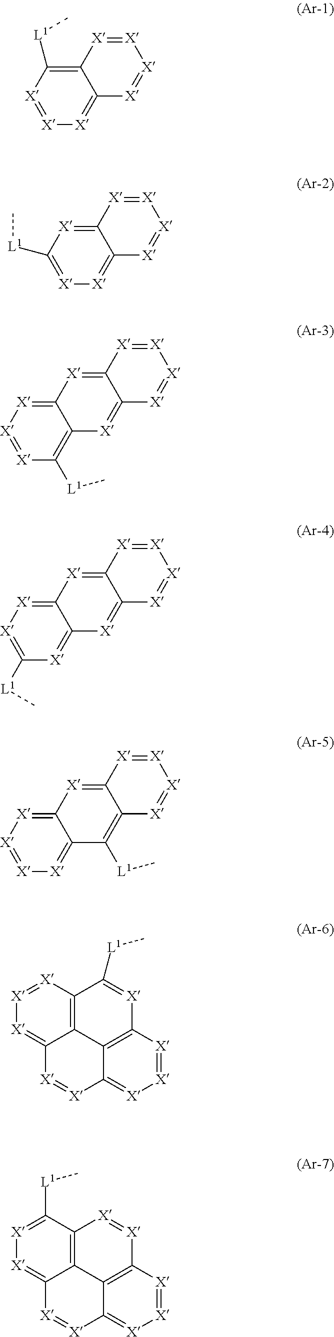

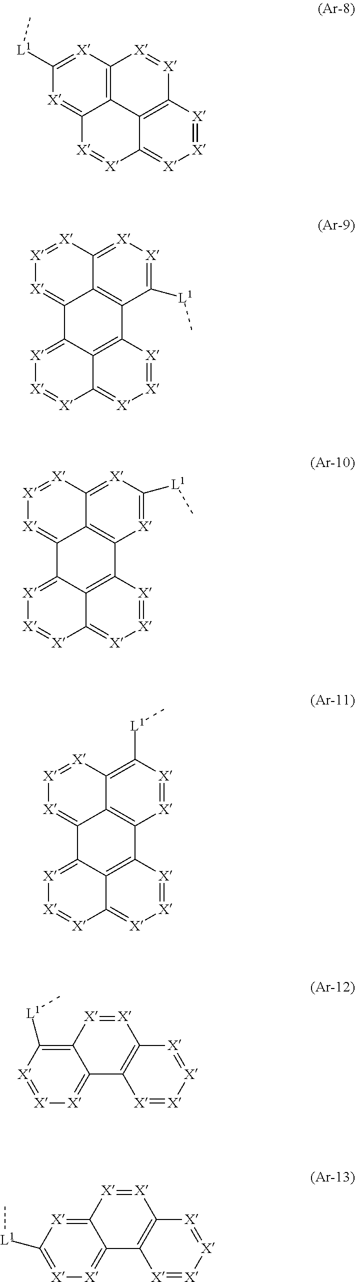

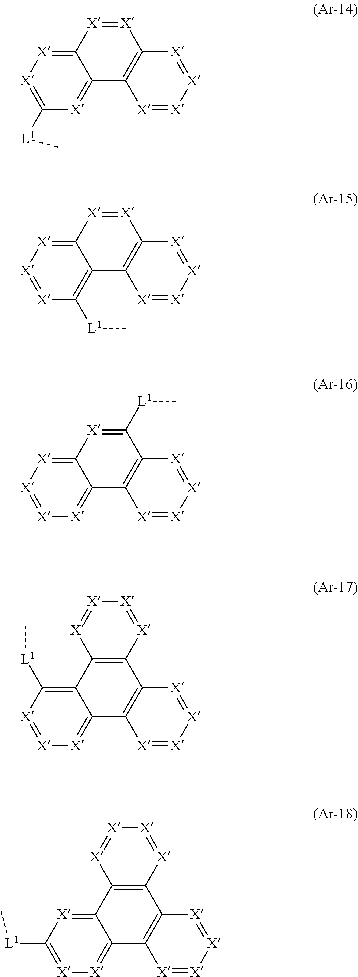

where X′ is N or CR^{a}, preferably CR^{a}, L¹ represents a bond or an aromatic or heteroaromatic ring system which has 5 to 40, preferably 5 to 30, aromatic ring atoms and may be substituted by one or more R¹ radicals, where the dotted bond marks the position of attachment, and in addition:

R^{a} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R^{a} radicals may also form a ring system with one another or a further group, for example with one or more of the R or R¹ radicals, where the symbols R¹ and Ar′ have the definitions given above, especially for formula (SE-I), (SE-II) and/or (SE-III).

It may preferably the case that, in formulae (Ar-1) to (Ar-18), not more than four, preferably not more than two and more preferably not more than one of the X′ group(s) is/are N, more preferably all X′ groups are CR^{a}.

In a particularly preferred configuration, it may the case that the aromatic or heteroaromatic ring system having two, preferably having three, fused aromatic or heteroaromatic rings is selected from the groups of the formulae (Ar′-1) to (Ar′-18)

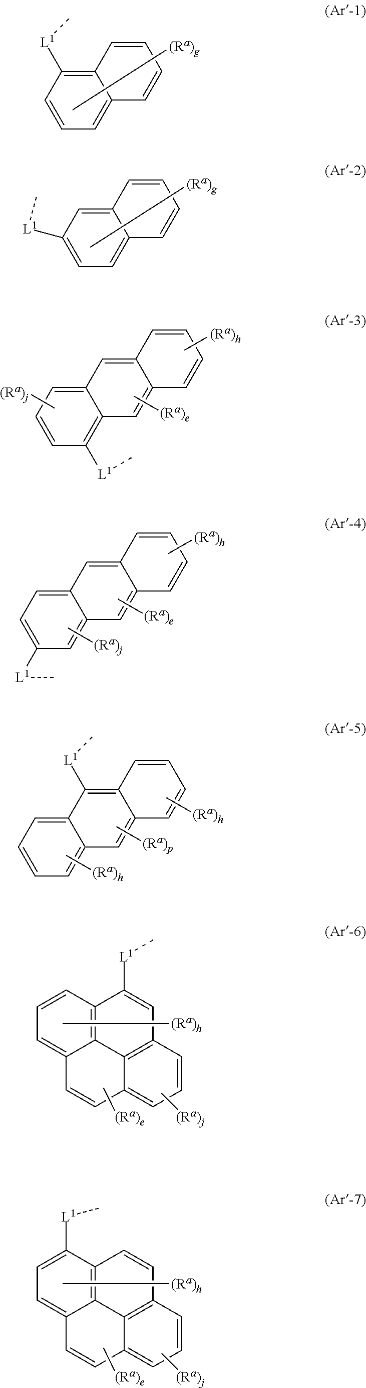

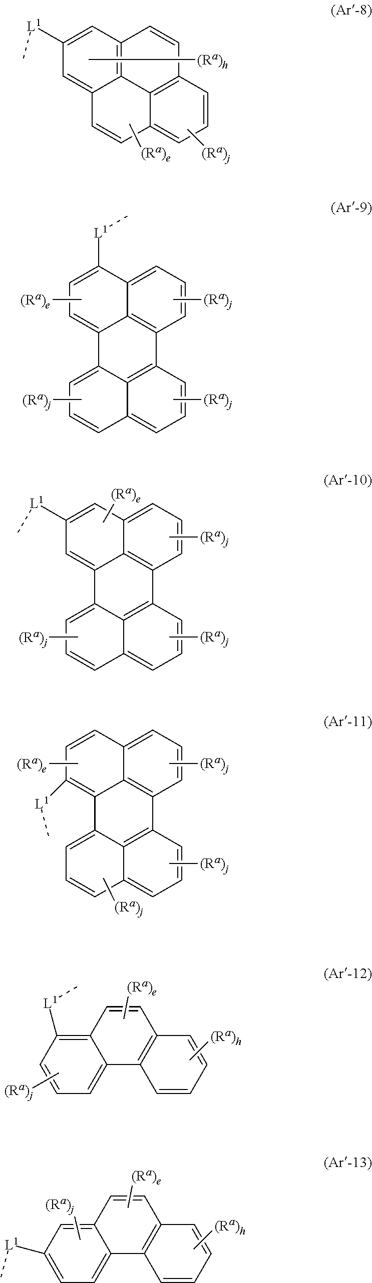

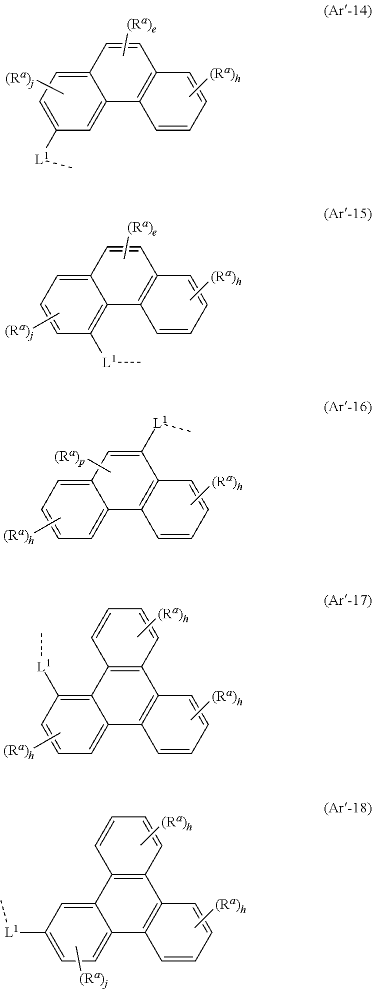

where L¹ represents a bond or an aromatic or heteroaromatic ring system which has 5 to 40, preferably 5 to 30, aromatic ring atoms and may be substituted by one or more R¹ radicals, where R¹ has the definition set out above, especially for formula (SE-I), (SE-II) and/or (SE-III), R^{a} has the definition set out above, especially for formulae (Ar-1) to (Ar-18), the dotted bond marks the position of attachment and the indices are as follows:
p is 0 or 1;
e is 0, 1 or 2, preferably 0 or 1;
j at each instance is independently 0, 1, 2 or 3, preferably 0, 1 or 2, more preferably preferably 0 or 1;
h at each instance is independently 0, 1, 2, 3 or 4, preferably 0, 1 or 2, more preferably preferably 0 or 1;
g is an integer in the range from 0 to 7, preferably 0, 1, 2, 3, 4, 5 or 6, particularly preferably 0, 1, 2, 3 or 4, especially preferably 0, 1 or 2.

It may further be the case that the sum total of the indices p, e, j, h and g in the structures of the formula (Ar-1) to (Ar-18) is not more than 3 in each case, preferably not more than 2 and more preferably not more than 1.

It may further be the case that the compound comprises at least one radical selected from the group of the phenyls, fluorenes, indenofluorenes, spirobifluorenes, carbazoles, indenocarbazoles, indolocarbazoles, spirocarbazoles, pyrimidines, triazines, quinazolines, quinoxalines, pyridines, quinolines, isoquinolines, lactams, triarylamines, dibenzofurans, dibenzothienes, imidazoles, benzimidazoles, benzoxazoles, benzthiazoles, 5-arylphenanthridin-6-ones, 9,10-dehydrophenanthrenes, fluoranthenes, naphthalenes, phenanthrenes, triphenylenes, anthracenes, benzanthracenes, fluoradenes, pyrenes, perylenes, chrysenes, borazines, boroxines, boroles, borazoles, azaboroles, ketones, phosphine oxides, arylsilanes, siloxanes and combinations thereof, where preferably at least one of the R^{a} and/or R radicals is selected from the aforementioned group.

It may further be the case that the compound comprises at least one radical selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 9,9′-diarylfluorenyl 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3-, 4- or 9-carbazolyl, 1- or 2-naphthyl, anthracenyl, preferably 9-anthracenyl, trans- and cis-indenofluorenyl, indenocarbazolyl, indolocarbazolyl, spirocarbazolyl, 5-aryl-phenanthridin-6-on-yl, 9,10-dehydrophenanthrenyl, fluoranthenyl, tolyl, mesityl, phenoxytolyl, anisolyl, triarylaminyl, bis(triarylaminyl), tris(triarylaminyl), hexamethylindanyl, tetralinyl, monocycloalkyl, biscycloalkyl, tricycloalkyl, alkyl, for example tert-butyl, methyl, propyl, alkoxyl, alkylsulfanyl, alkylaryl, triarylsilyl, trialkylsilyl, xanthenyl, 10-arylphenoxazinyl, phenanthrenyl and/or triphenylenyl, each of which may be substituted by one or more radicals, but are preferably unsubstituted, particular preference being given to phenyl, spirobifluorene, fluorene, dibenzofuran, dibenzothiophene, anthracene, phenanthrene, triphenylene groups, where preferably at least one of the R^{a} and/or R radicals is selected from the aforementioned group.

It may additionally be the case that the compound contains one or more crosslinkable groups.

It may preferably be the case that the compound has a molecular weight of not more than 5000 g/mol, preferably not more than 4000 g/mol, particularly preferably not more than 3000 g/mol, especially preferably not more than 2000 g/mol and most preferably not more than 1200 g/mol.

In a preferred embodiment, the compound preferably has a glass transition temperature of at least 100° C., more preferably of at least 120° C., even more preferably of at least 150° C. and especially preferably of at least 180° C., determined in accordance with DIN 51005.

The present invention further provides novel compounds with which functional layers can be structured and are of excellent suitability for production of improved electronic devices.

The present invention therefore further provides a compound comprising at least one structure of the formula (I), preferably a compound of formula (I),

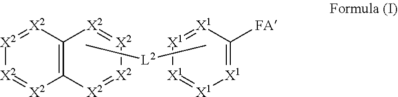

where the FA′ group is a fluorinated alkyl radical which has at least two carbon atoms and may be substituted by one or more R radicals, but is preferably unsubstituted, where the symbol R has the definition given above, especially for formula (SE-I), (SE-II) and/or (SE-III), and the further symbols are as follows:
X¹ is CR^{b}, N or C if the L² group binds to X¹, preferably CR^{b} or C;
X² is CR^{c}, N or C if the L² group binds to X², preferably CR^{c} or C;
L² is a linking group, preferably a bond or an aromatic or heteroaromatic ring system which has 5 to 40, preferably 5 to 30, aromatic ring atoms and may be substituted by one or more R¹ radicals, where the symbol R¹ has the definition given above, especially for formula (SE-I), (SE-II) and/or (SE-III),
R^{b} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, CEC, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R^{b} radicals may also form a ring system with one another or a further group, for example with one or more of the R^{c} radicals, where the symbols R¹ and Ar have the definition set out above, especially for formula (SE-I), (SE-II) and/or (SE-III);
R^{c} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —O(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R^{c} radicals may also form a ring system with one another or a further group, for example with one or more of the R^{b} radicals, where the symbols R¹ and Ar have the definition set out above, especially for formula (SE-I), (SE-II) and/or (SE-III).

In a further-preferred configuration, it may be the case that the compounds of the invention comprise at least one structure of the formulae (I-1) to (I-7), where the compounds of the invention may more preferably be selected from the compounds of the formulae (I-1) to (I-7)

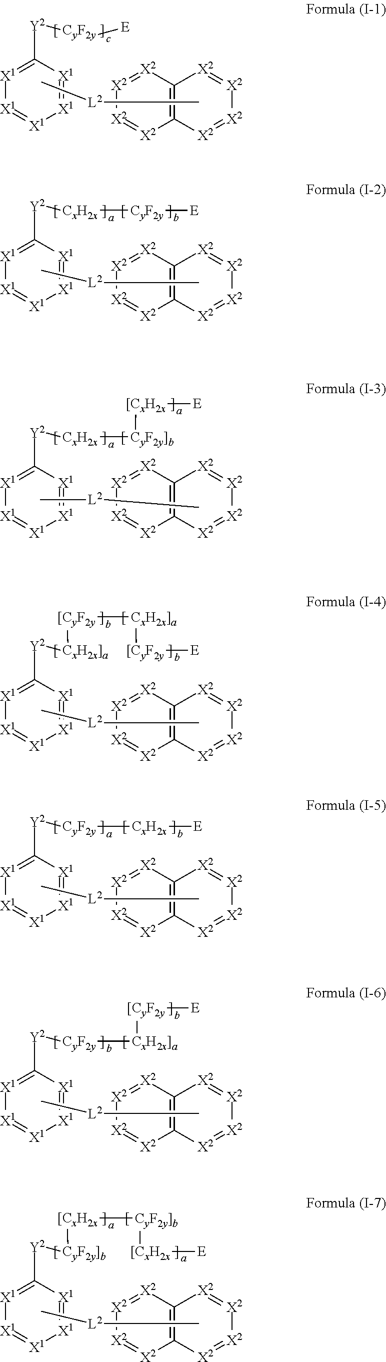

where the symbols L², X¹ and X² have the definition given above, especially for formula (I), and the further symbols are as follows:
E is selected from H, D or F, preferably H or F;
Y² is the same or different at each instance and is a bond, O, S, NR⁴ or C(═O), preferably a bond, O, S, NR⁴, more preferably a bond, O or S, especially preferably a bond;
R⁴ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, the R⁴ radical may form a ring system together with another part of the compound, where the symbol R² has the definition set out above, especially for formula (SE-I), (SE-II) and/or (SE-III),
a is the same or different at each instance and is 1, 2, 3, 4, 5 or 6, preferably 1, 2, 3 or 4, more preferably 1 or 2;
b is the same or different at each instance and is 1, 2, 3, 4, 5 or 6, preferably 1, 2, 3 or 4, more preferably 1 or 2;
c is 2, 3, 4, 5, 6, 7, 8, 9 or 10, preferably 2, 3, 4, 5 or 6, more preferably 2, 3 or 4, most preferably 2 or 3;
x is the same or different at each instance and is 1, 2, 3, 4, 5 or 6, preferably 1, 2, 3 or 4, more preferably 1 or 2;
y is the same or different at each instance and is 1, 2, 3, 4, 5 or 6, preferably 1, 2, 3 or 4, more preferably 1 or 2.

In a further-preferred configuration, it may be the case that the compounds of the invention comprise a structure of the formulae (Ia-1) to (Ia-7), where the compounds of the invention may more preferably be selected from the compounds of the formulae (Ia-1) to (Ia-7),

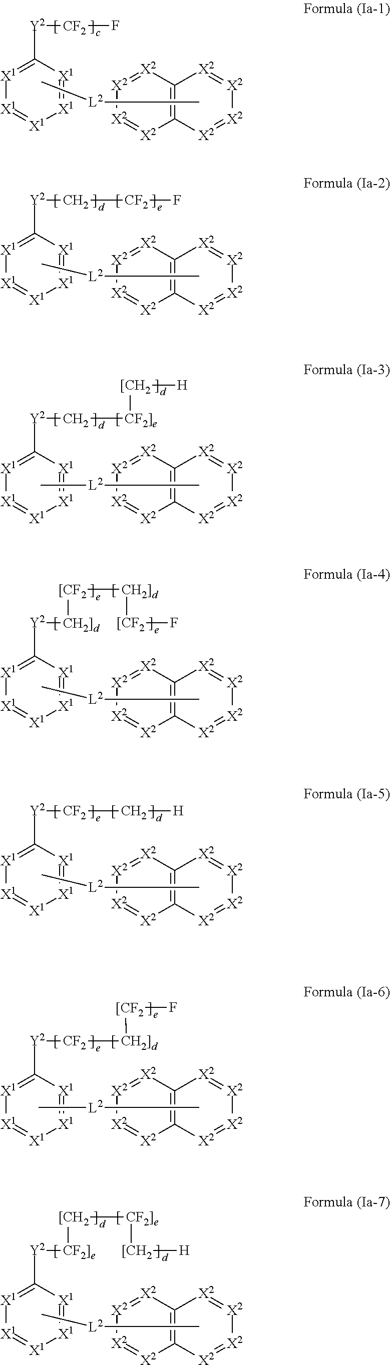

where the symbols L², X¹ and X² have the definitions given above, especially for formula (I), the symbol Y² and the index c have the definitions given above, especially for formulae (I-1) to (I-7), and in addition:
d is the same or different at each instance and is 1, 2, 3, 4, 5, 6, 7, 8, 9 or 10, preferably 1, 2, 3, 4, 5 or 6, more preferably 1, 2, 3 or 4, most preferably 1 or 2;
e is the same or different at each instance and is 1, 2, 3, 4, 5, 6, 7, 8, 9 or 10, preferably 1, 2, 3, 4, 5 or 6, more preferably 1, 2, 3 or 4, most preferably 1 or 2.

In a further-preferred configuration, it may be the case that the compounds of the invention comprise a structure of the formulae (Ib-1) to (Ib-7), where the compounds of the invention may more preferably be selected from the compounds of the formulae (Ib-1) to (Ib-7),

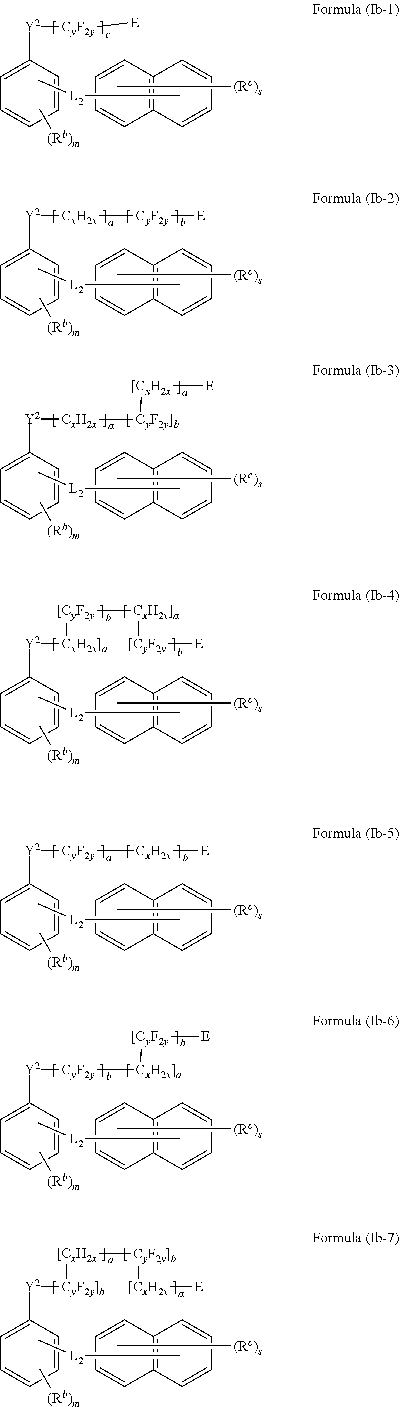

where the symbols L², R^{b} and R^{c} have the definitions given above, especially for formula (I), the symbols Y² and E and the indices a, b, c, x and y have the definitions given above, especially for formulae (I-1) to (I-7), the index s is 0, 1, 2, 3, 4, 5, 6 or 7, preferably 0, 1, 2, 3 or 4, more preferably 0, 1 or 2; and the index m is 0, 1, 2, 3 or 4, preferably 0, 1 or 2, more preferably 0 or 1.

In a further-preferred configuration, it may be the case that the compounds of the invention comprise a structure of the formulae (Ic-1) to (Ic-7), where the compounds of the invention may more preferably be selected from the compounds of the formulae (Ic-1) to (Ic-7),

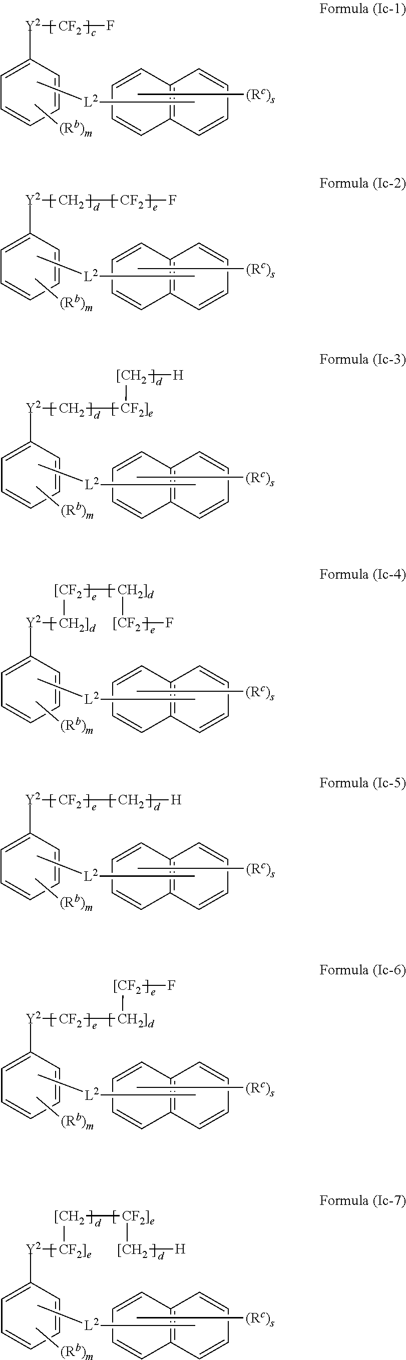

where the symbols L², R^{b} and R^{c} have the definitions given above, especially for formula (I), the symbol Y² and the index c have the definitions given above, especially for formulae (I-1) to (I-7), the indices d and e have the definitions given above, especially for formulae (Ia-1) to (Ia-7), the index s is 0, 1, 2, 3, 4, 5, 6 or 7, preferably 0, 1, 2, 3 or 4, more preferably 0, 1 or 2; and the index m is 0, 1, 2, 3 or 4, preferably 0, 1 or 2, more preferably 0 or 1.

The present invention preferably further provides a compound comprising at least one structure of the formula (II), preferably a compound of formula (II),

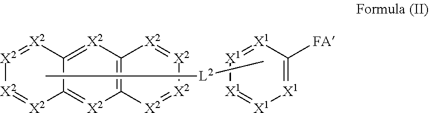

where the FA′ group is a fluorinated alkyl radical which has at least two carbon atoms and may be substituted by one or more R radicals, but is preferably unsubstituted, where symbol R has the definition given above, especially for formula (SE-I), (SE-II) and/or (SE-III), and the symbols L², X¹ and X² have the definitions given above, especially for formula (I).

In a further-preferred configuration, it may be the case that the compounds of the invention comprise at least one structure of the formulae (II-1) to (II-7), where the compounds of the invention may more preferably be selected from the compounds of the formulae (II-1) to (II-7)

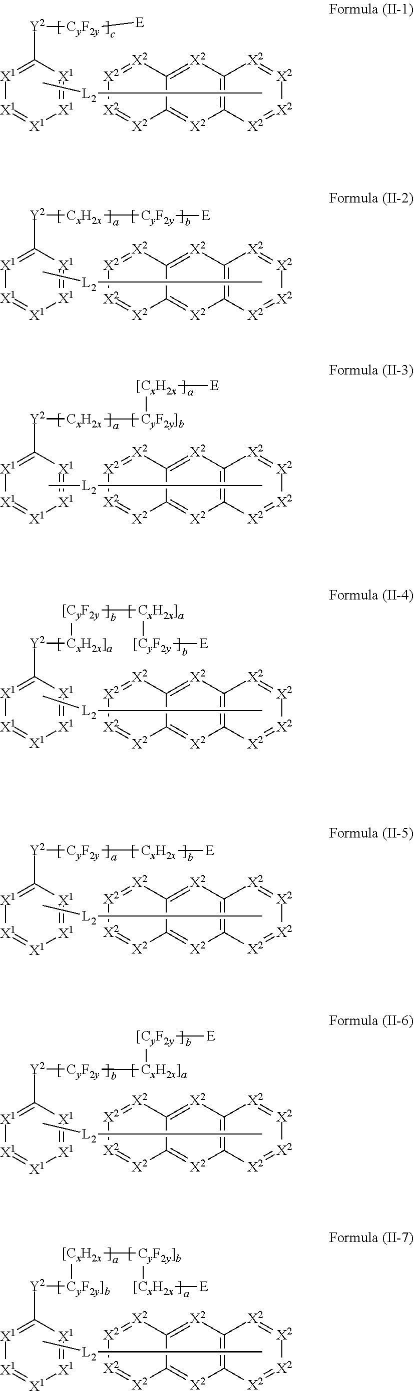

where the symbols L², X¹ and X² have the definitions given above, especially for formula (I), the symbols Y² and E and the indices a, b, c, x and y have the definitions given above, especially for formulae (I-1) to (I-7).

In a further-preferred configuration, it may be the case that the compounds of the invention comprise a structure of the formulae (IIa-1) to (IIa-7), where the compounds of the invention may more preferably be selected from the compounds of the formulae (IIa-1) to (IIa-7),

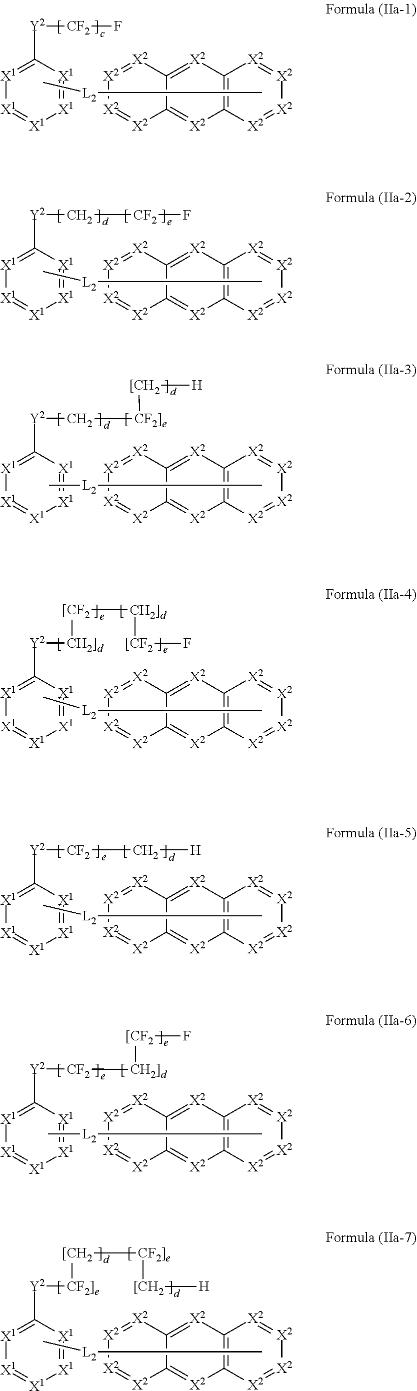

where the symbols L², X¹ and X² have the definitions given above, especially for formula (I), the symbol Y² and the index c have the definitions given above, especially for formulae (I-1) to (I-7), the indices d and e have the definitions given above, especially for formulae (Ia-1) to (Ia-7).

In a further-preferred configuration, it may be the case that the compounds of the invention comprise a structure of the formulae (IIb-1) to (IIb-7), where the compounds of the invention may more preferably be selected from the compounds of the formulae (IIb-1) to (IIb-7),

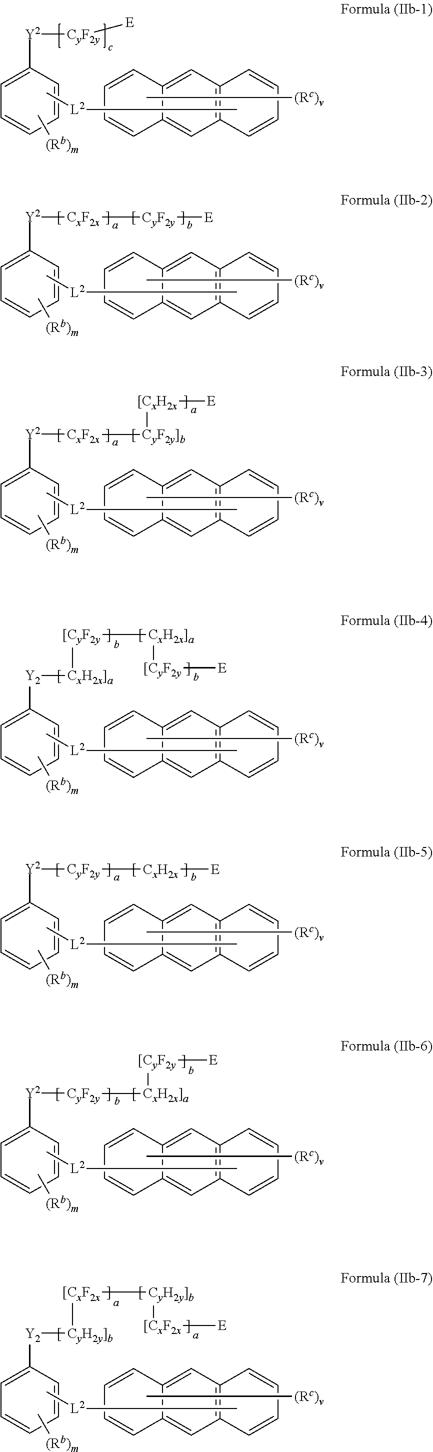

where the symbols L², R^{b} and R^{c} have the definitions given above, especially for formula (I), the symbols Y² and E and the indices a, b, c, x and y have the definitions given above, especially for formulae (I-1) to (I-7), the index v is 0, 1, 2, 3, 4, 5, 6, 7, 8 or 9, preferably 0, 1, 2, 3 or 4, more preferably 0, 1 or 2; and the index m is 0, 1, 2, 3 or 4, preferably 0, 1 or 2, more preferably 0 or 1.

In a further-preferred configuration, it may be the case that the compounds of the invention comprise a structure of the formulae (IIc-1) to (IIc-7), where the compounds of the invention may more preferably be selected from the compounds of the formulae (IIc-1) to (IIc-7),

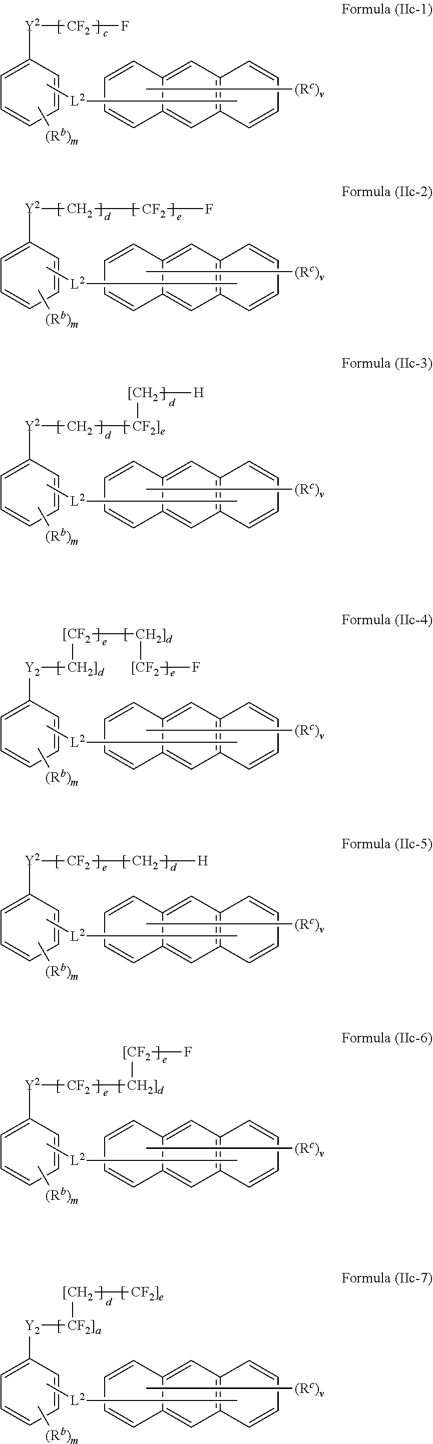

where the symbols L², R^{b} and R^{c} have the definitions given above, especially for formula (I), the symbol Y² and the index c have the definitions given above, especially for formulae (I-1) to (I-7), the indices d and e have the definitions given above, especially for formulae (Ia-1) to (Ia-7), the index v is 0, 1, 2, 3, 4, 5, 6, 7, 8 or 9, preferably 0, 1, 2, 3 or 4, more preferably 0, 1 or 2; and the index m is 0, 1, 2, 3 or 4, preferably 0, 1 or 2, more preferably 0 or 1.

The present invention preferably further provides a compound comprising at least one structure of the formula (III), preferably a compound of formula (III),

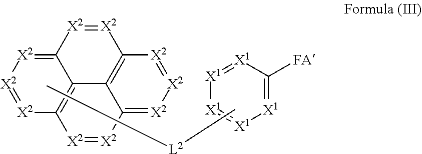

where the FA′ group is a fluorinated alkyl radical which has at least two carbon atoms and may be substituted by one or more R radicals, but is preferably unsubstituted, where the symbol R has the definition given above, especially for formula (SE-I), (SE-II) and/or (SE-III), and the symbols L², X¹ and X² have the definitions given above, especially for formula (I).

In a further-preferred configuration, it may be the case that the compounds of the invention comprise at least one structure of the formulae (III-1) to (III-7), where the compounds of the invention may more preferably be selected from the compounds of the formulae (III-1) to (III-7)

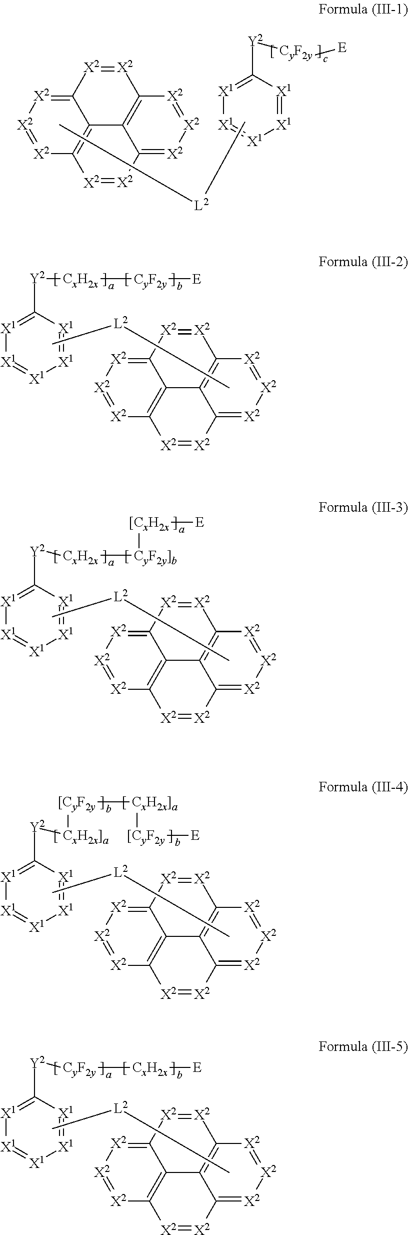

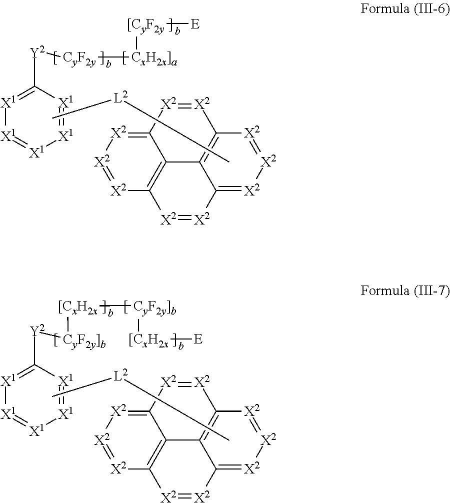

where the symbols L², X¹ and X² have the definitions given above, especially for formula (I), the symbols Y² and E and the indices a, b, c, x and y have the definitions given above, especially for formulae (I-1) to (I-7).

In a further-preferred configuration, it may be the case that the compounds of the invention comprise a structure of the formulae (IIIa-1) to (IIIa-7), where the compounds of the invention may more preferably be selected from the compounds of the formulae (IIIa-1) to (IIIa-7),

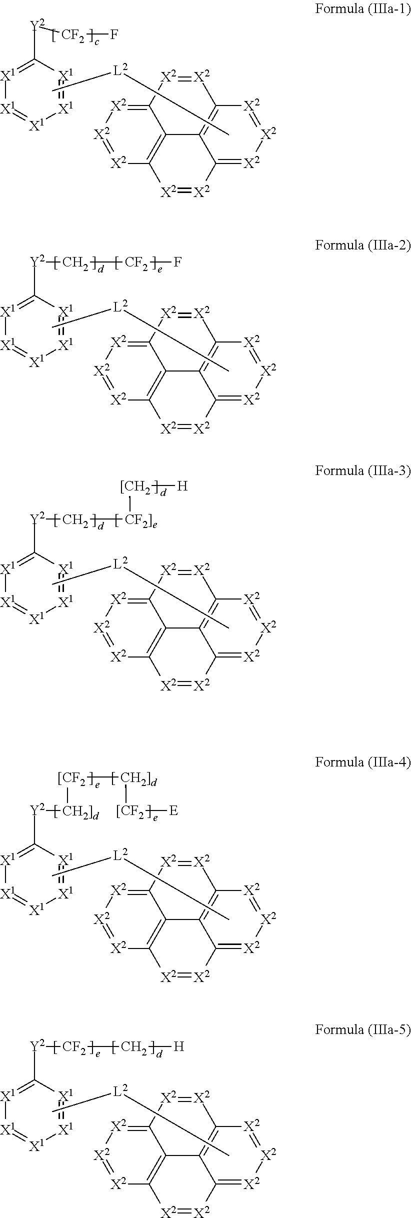

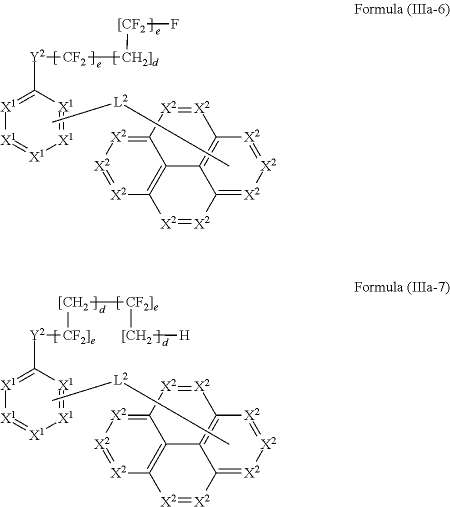

where the symbols L², X¹ and X² have the definitions given above, especially for formula (I), the symbol Y² and the index c have the definitions given above, especially for formulae (I-1) to (I-7), the indices d and e have the definitions given above, especially for formulae (Ia-1) to (Ia-7).

In a further-preferred configuration, it may be the case that the compounds of the invention comprise a structure of the formulae (IIIb-1) to (IIIb-7), where the compounds of the invention may more preferably be selected from the compounds of the formulae (IIIb-1) to (IIIb-7),

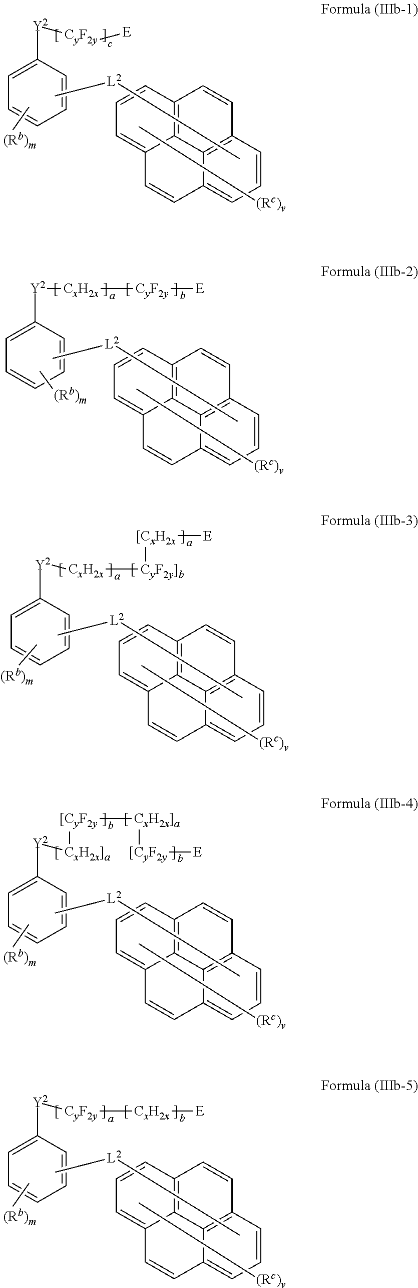

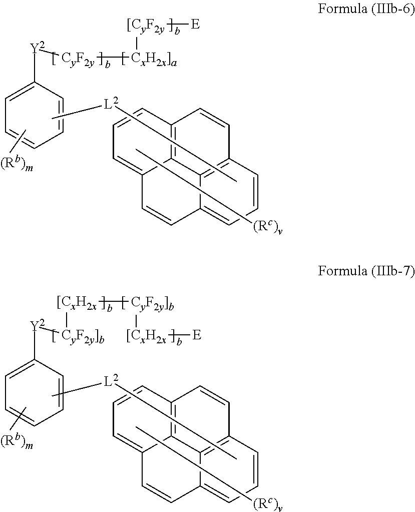

where the symbols L², R^{b} and R^{c} have the definitions given above, especially for formula (I), the symbols Y² and E and the indices a, b, c, x and y have the definitions given above, especially for formulae (I-1) to (I-7), the index v is 0, 1, 2, 3, 4, 5, 6, 7, 8 or 9, preferably 0, 1, 2, 3 or 4, more preferably 0, 1 or 2; and the index m is 0, 1, 2, 3 or 4, preferably 0, 1 or 2, more preferably 0 or 1.

In a further-preferred configuration, it may be the case that the compounds of the invention comprise a structure of the formulae (IIIc-1) to (IIIc-7), where the compounds of the invention may more preferably be selected from the compounds of the formulae (IIIc-1) to (IIIc-7),

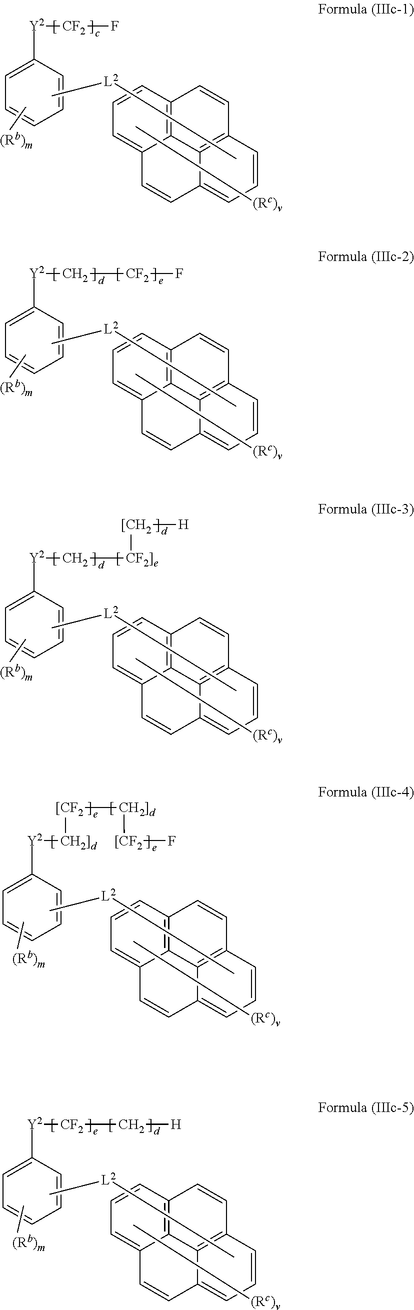

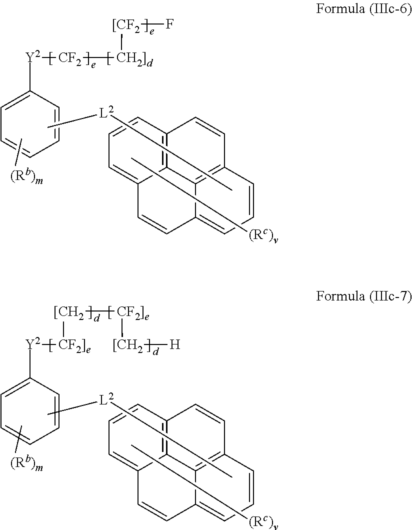

where the symbols L², R^{b} and R^{c} have the definitions given above, especially for formula (I), the symbol Y² and the index c have the definitions given above, especially for formulae (I-1) to (I-7), the indices d and e have the definitions given above, especially for formulae (Ia-1) to (Ia-7), the index v is 0, 1, 2, 3, 4, 5, 6, 7, 8 or 9, preferably 0, 1, 2, 3 or 4, more preferably 0, 1 or 2; and the index m is 0, 1, 2, 3 or 4, preferably 0, 1 or 2, more preferably 0 or 1.

The present invention preferably further provides a compound comprising at least one structure of the formula (IV), preferably a compound of formula (IV),

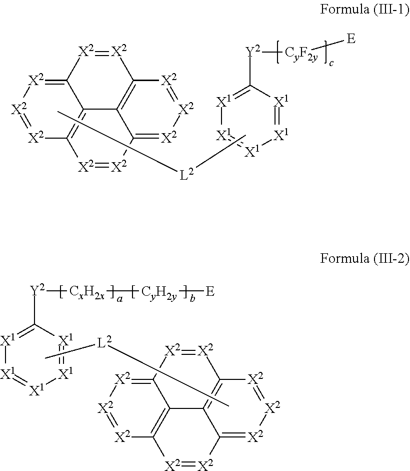

where the FA′ group is a fluorinated alkyl radical which has at least two carbon atoms and may be substituted by one or more R radicals, but is preferably unsubstituted, where the symbol R has the definition given above, especially for formula (SE-I), (SE-II) and/or (SE-III), and the symbols L², X¹ and X² have the definitions given above, especially for formula (I).

In a further-preferred configuration, it may be the case that the compounds of the invention comprise at least one structure of the formulae (IV-1) to (IV-7), where the compounds of the invention may more preferably be selected from the compounds of the formulae (IV-1) to (IV-7)

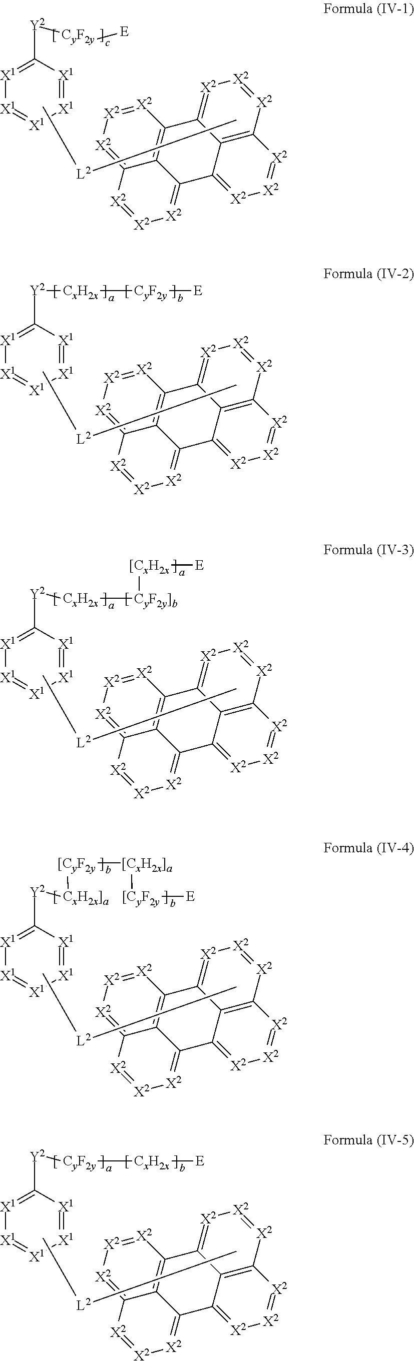

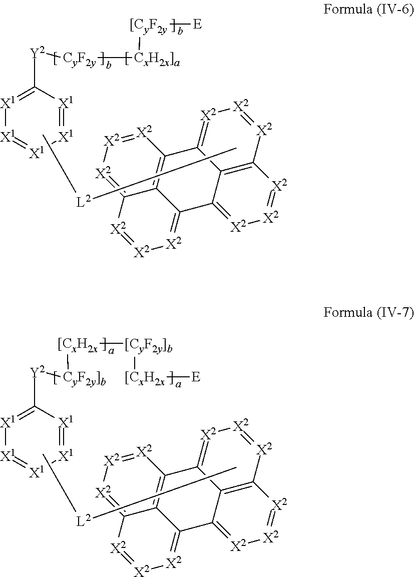

where the symbols L², X¹ and X² have the definitions given above, especially for formula (I), the symbols Y² and E and the indices a, b, c, x and y have the definitions given above, especially for formulae (I-1) to (I-7).

In a further-preferred configuration, it may be the case that the compounds of the invention comprise a structure of the formulae (IVa-1) to (IVa-7), where the compounds of the invention may more preferably be selected from the compounds of the formulae (IVa-1) to (IVa-7),

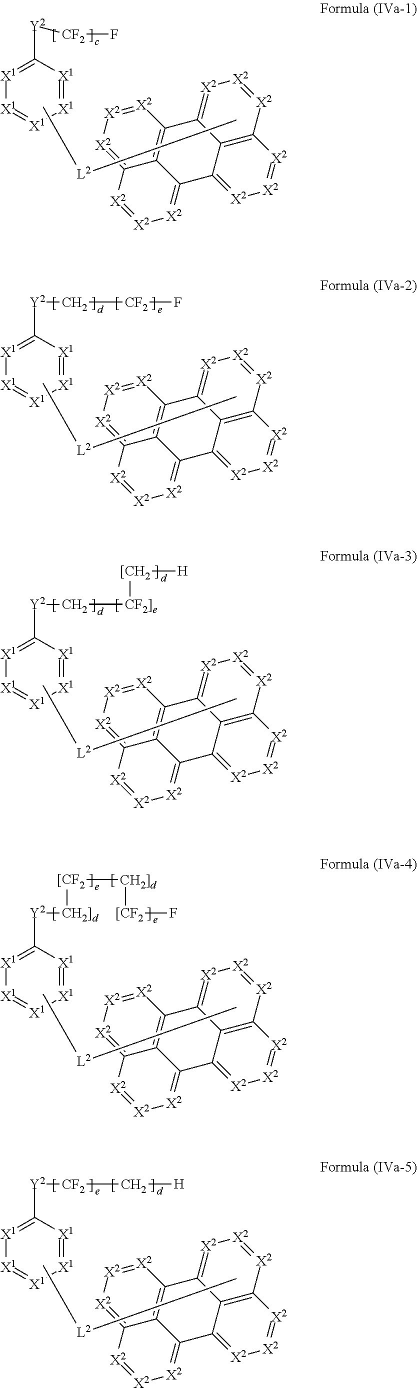

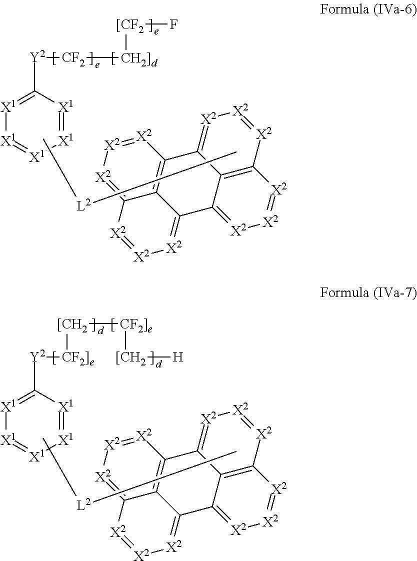

where the symbols L², X¹ and X² have the definitions given above, especially for formula (I), the symbol Y² and the index c have the definitions given above, especially for formulae (I-1) to (I-7), the indices d and e have the definitions given above, especially for formulae (Ia-1) to (Ia-7).

In a further-preferred configuration, it may be the case that the compounds of the invention comprise a structure of the formulae (IVb-1) to (IVb-7), where the compounds of the invention may more preferably be selected from the compounds of the formulae (IVb-1) to (IVb-7),

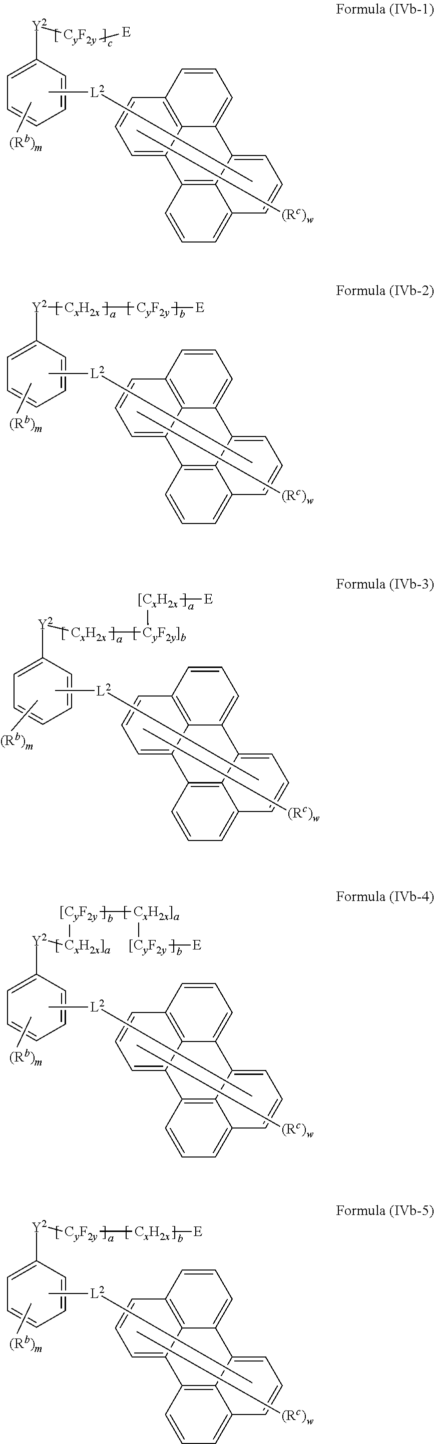

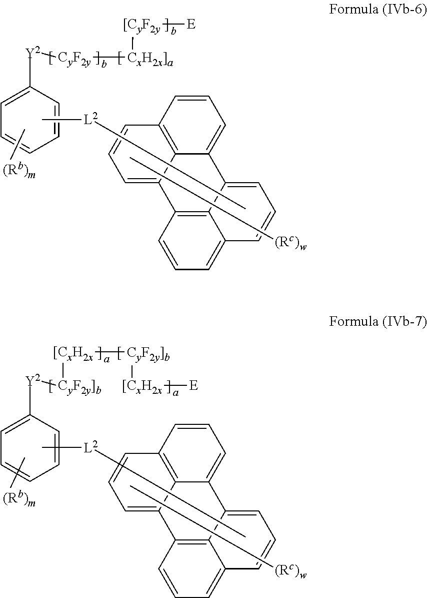

where the symbols L², R^{b} and R^{c} have the definitions given above, especially for formula (I), the symbols Y² and E and the indices a, b, c, x and y have the definitions given above, especially for formulae (I-1) to (I-7), the index w is 0, 1, 2, 3, 4, 5, 6, 7, 8, 9, 10 or 11, preferably 0, 1, 2, 3 or 4, more preferably 0, 1 or 2; and the index m is 0, 1, 2, 3 or 4, preferably 0, 1 or 2, more preferably 0 or 1.

In a further-preferred configuration, it may be the case that the compounds of the invention comprise a structure of the formulae (IVc-1) to (IVc-7), where the compounds of the invention may more preferably be selected from the compounds of the formulae (IVc-1) to (IVc-7),

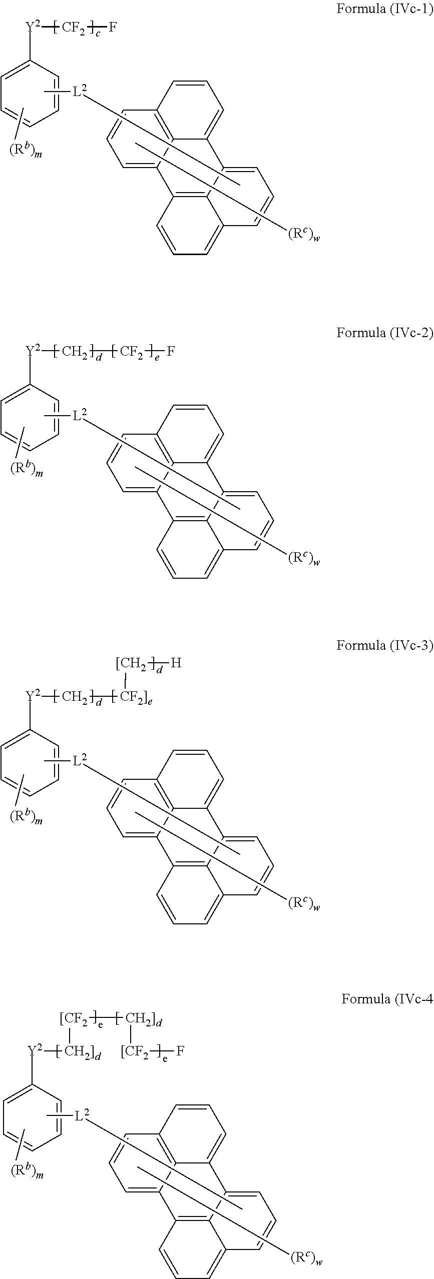

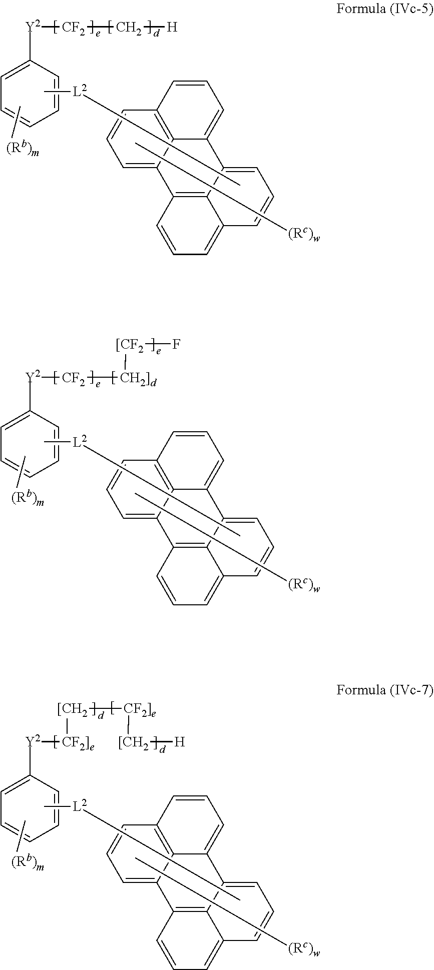

where the symbols L², R^{b} and R^{c} have the definitions given above, especially for formula (I), the symbol Y² and the index c have the definitions given above, especially for formulae (I-1) to (I-7), the indices d and e have the definitions given above, especially for formulae (Ia-1) to (Ia-7), the index w is 0, 1, 2, 3, 4, 5, 6, 7, 8, 9, 10 or 11, preferably 0, 1, 2, 3 or 4, more preferably 0, 1 or 2; and the index m is 0, 1, 2, 3 or 4, preferably 0, 1 or 2, more preferably 0 or 1.

The present invention preferably further provides a compound comprising at least one structure of the formula (V), preferably a compound of formula (V),

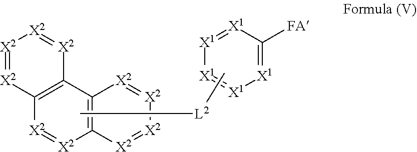

where the FA′ group is a fluorinated alkyl radical which has at least two carbon atoms and may be substituted by one or more R radicals, but is preferably unsubstituted, where the symbol R has the definition given above, especially for formula (SE-I), (SE-II) and/or (SE-III), and the symbols L², X¹ and X² have the definitions given above, especially for formula (I).

In a further-preferred configuration, it may be the case that the compounds of the invention comprise at least one structure of the formulae (V-1) to (V-7), where the compounds of the invention may more preferably be selected from the compounds of the formulae (V-1) to (V-7)

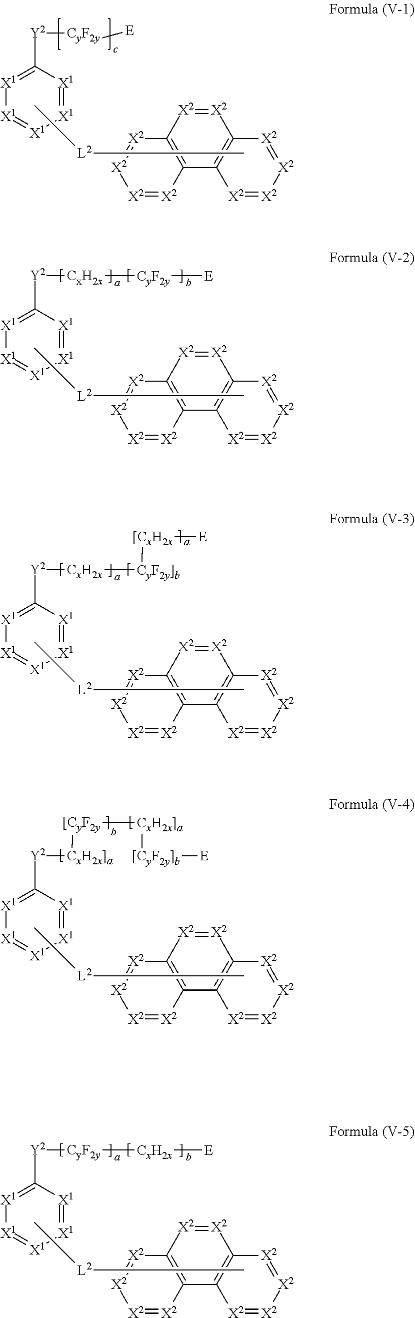

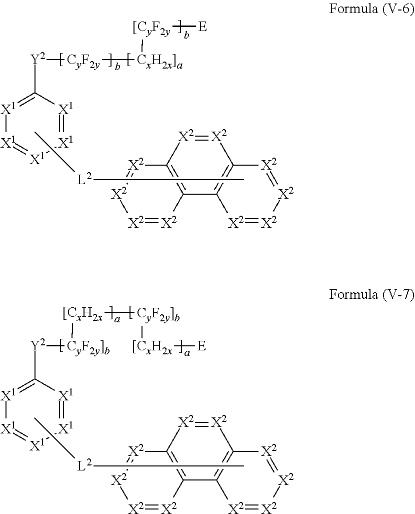

where the symbols L², X¹ and X² have the definitions given above, especially for formula (I), the symbols Y² and E and the indices a, b, c, x and y have the definitions given above, especially for formulae (I-1) to (I-7).

In a further-preferred configuration, it may be the case that the compounds of the invention comprise a structure of the formulae (Va-1) to (Va-7), where the compounds of the invention may more preferably be selected from the compounds of the formulae (Va-1) to (Va-7),

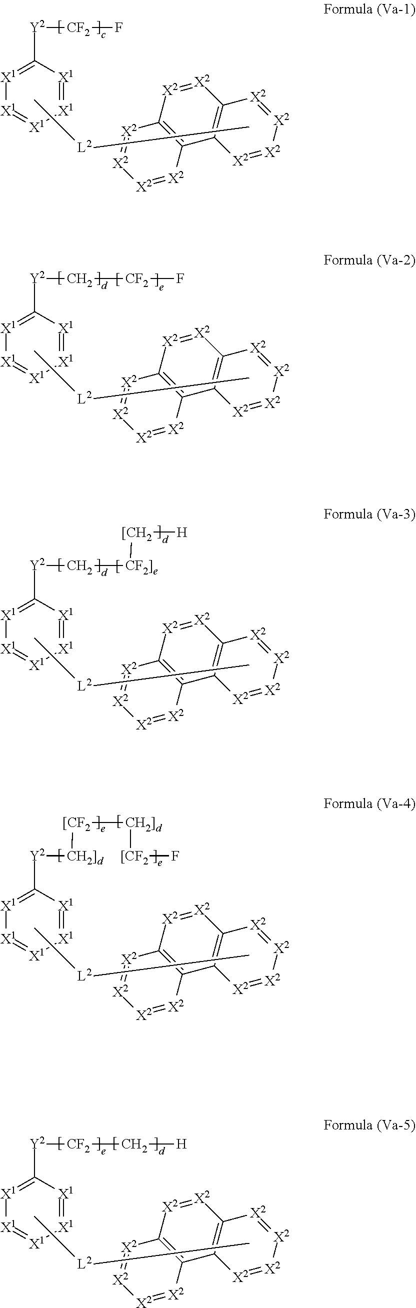

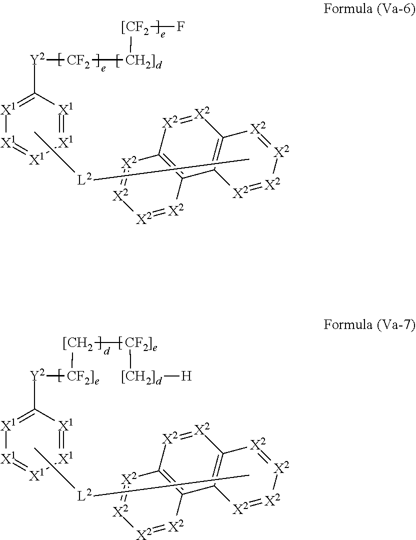

where the symbols L², X¹ and X² have the definitions given above, especially for formula (I), the symbol Y² and the index c have the definitions given above, especially for formulae (I-1) to (I-7), the indices d and e have the definitions given above, especially for formulae (Ia-1) to (Ia-7).

In a further-preferred configuration, it may be the case that the compounds of the invention comprise a structure of the formulae (Vb-1) to (Vb-7), where the compounds of the invention may more preferably be selected from the compounds of the formulae (Vb-1) to (Vb-7),

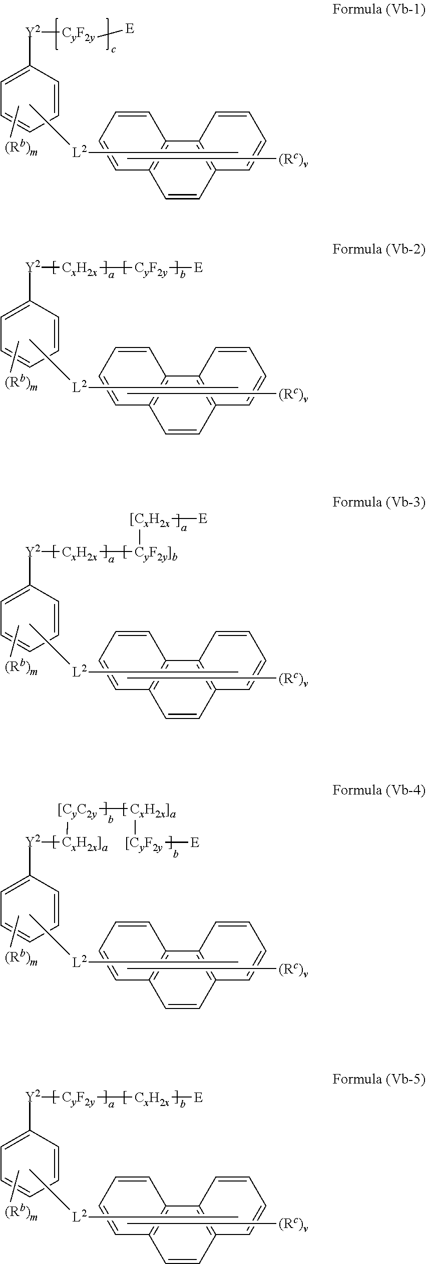

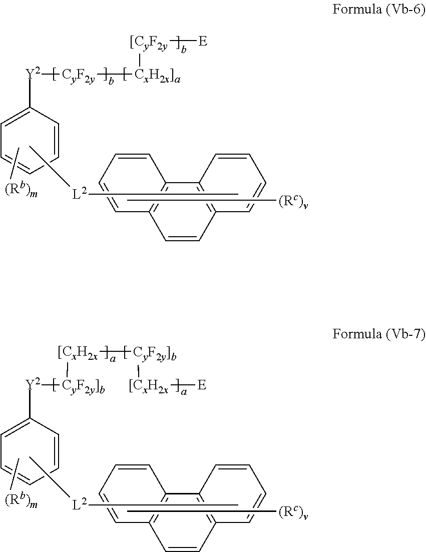

where the symbols L², R^{b} and R^{c} have the definitions given above, especially for formula (I), the symbols Y² and E and the indices a, b, c, x and y have the definitions given above, especially for formulae (I-1) to (I-7), the index v is 0, 1, 2, 3, 4, 5, 6, 7, 8 or 9, preferably 0, 1, 2, 3 or 4, more preferably 0, 1 or 2; and the index m is 0, 1, 2, 3 or 4, preferably 0, 1 or 2, more preferably 0 or 1.

In a further-preferred configuration, it may be the case that the compounds of the invention comprise a structure of the formulae (Vc-1) to (Vc-7), where the compounds of the invention may more preferably be selected from the compounds of the formulae (Vc-1) to (Vc-7),

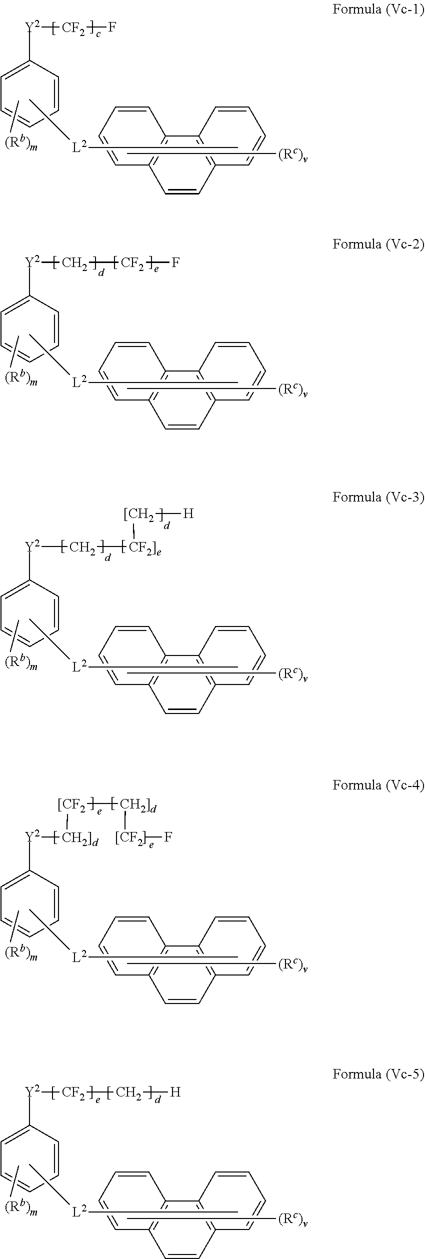

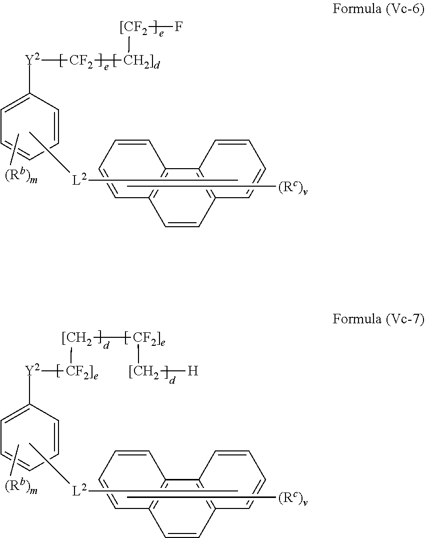

where the symbols L², R^{b} and R^{c} have the definitions given above, especially for formula (I), the symbol Y² and the index c have the definitions given above, especially for formulae (I-1) to (I-7), the indices d and e have the definitions given above, especially for formulae (Ia-1) to (Ia-7), the index v is 0, 1, 2, 3, 4, 5, 6, 7, 8 or 9, preferably 0, 1, 2, 3 or 4, more preferably 0, 1 or 2; and the index m is 0, 1, 2, 3 or 4, preferably 0, 1 or 2, more preferably 0 or 1.

The present invention preferably additionally provides a compound comprising at least one structure of the formula (VI), preferably a compound of formula (VI),

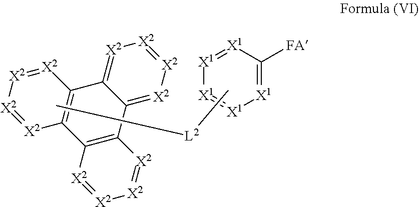

where the FA′ group is a fluorinated alkyl radical which has at least two carbon atoms and may be substituted by one or more R radicals, but is preferably unsubstituted, where the symbol R has the definition given above, especially for formula (SE-I), (SE-II) and/or (SE-III), and the symbols L², X¹ and X² have the definitions given above, especially for formula (I).

In a further-preferred configuration, it may be the case that the compounds of the invention comprise at least one structure of the formulae (VI-1) to (VI-7), where the compounds of the invention may more preferably be selected from the compounds of the formulae (VI-1) to (VI-7)

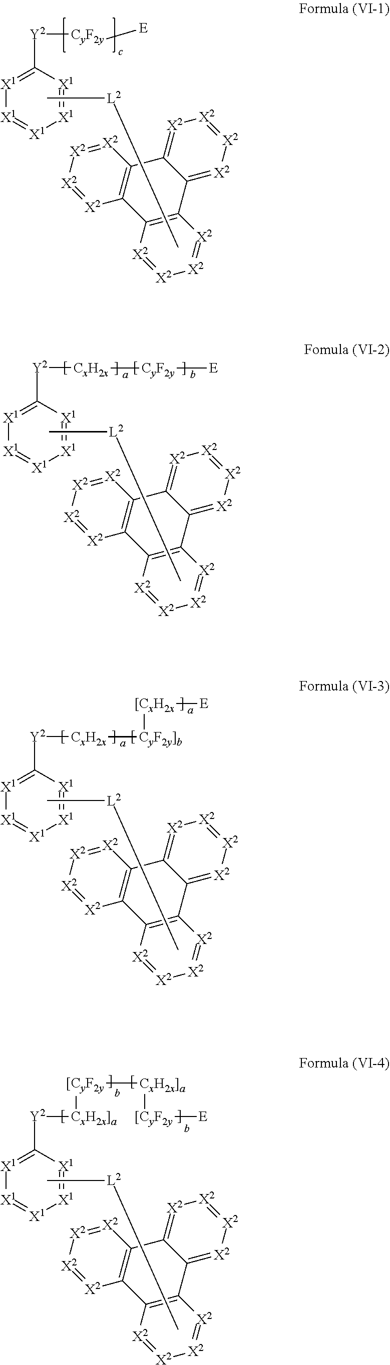

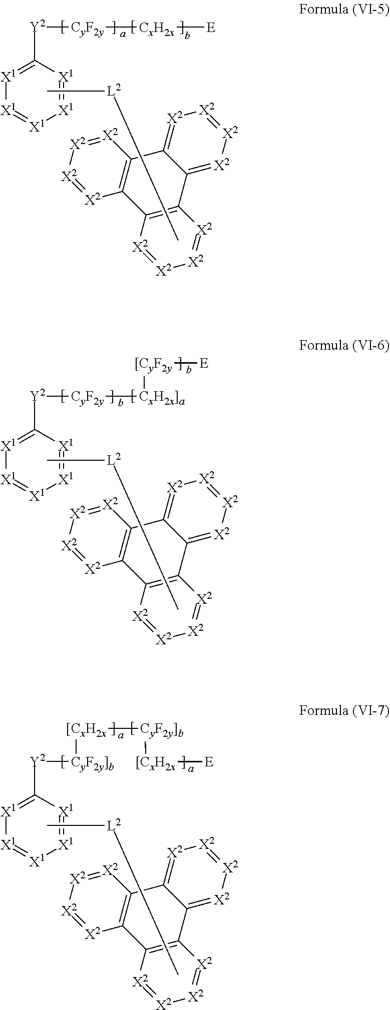

where the symbols L², X¹ and X² have the definitions given above, especially for formula (I), the symbols Y² and E and the indices a, b, c, x and y have the definitions given above, especially for formulae (I-1) to (I-7).

In a further-preferred configuration, it may be the case that the compounds of the invention comprise a structure of the formulae (VIa-1) to (VIa-7), where the compounds of the invention may more preferably be selected from the compounds of the formulae (VIa-1) to (VIa-7),

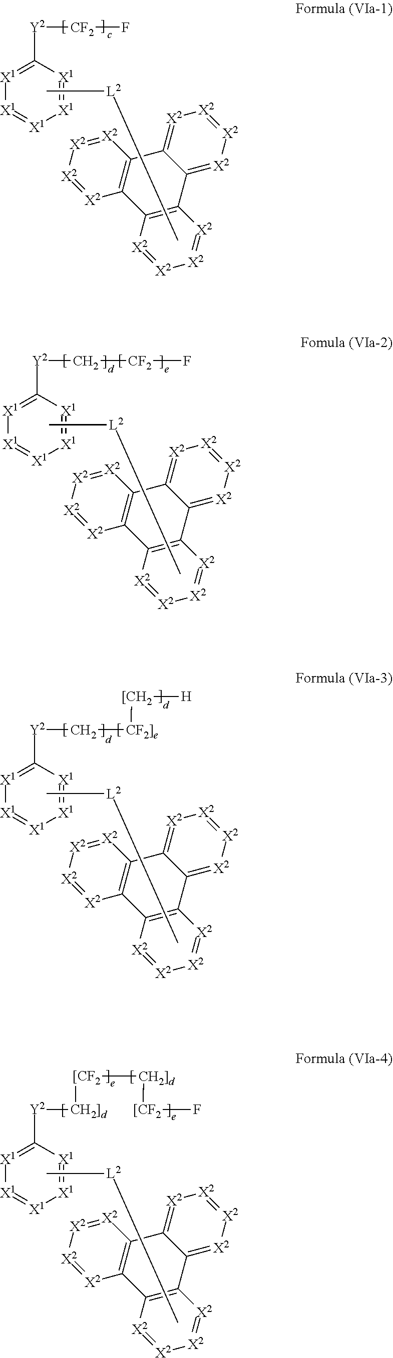

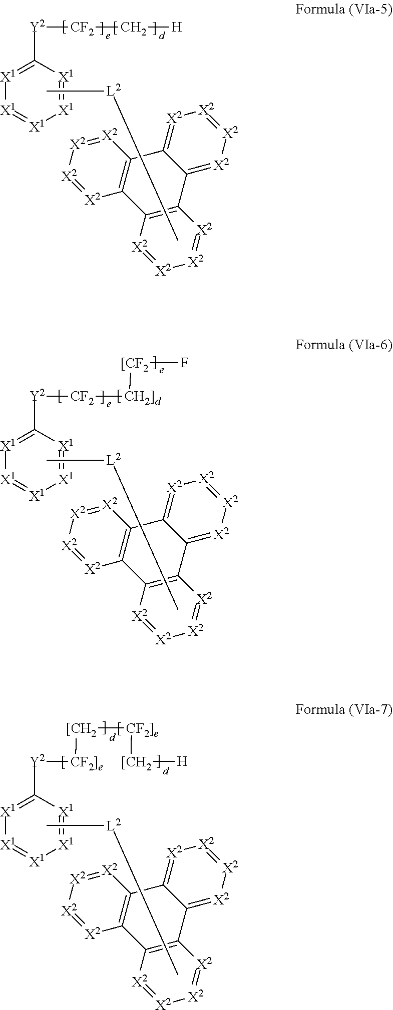

where the symbols L², X¹ and X² have the definitions given above, especially for formula (I), the symbol Y² and the index c have the definitions given above, especially for formulae (I-1) to (I-7), the indices d and e have the definitions given above, especially for formulae (Ia-1) to (Ia-7).

In a further-preferred configuration, it may be the case that the compounds of the invention comprise a structure of the formulae (VIb-1) to (VIb-7), where the compounds of the invention may more preferably be selected from the compounds of the formulae (VIb-1) to (VIb-7),

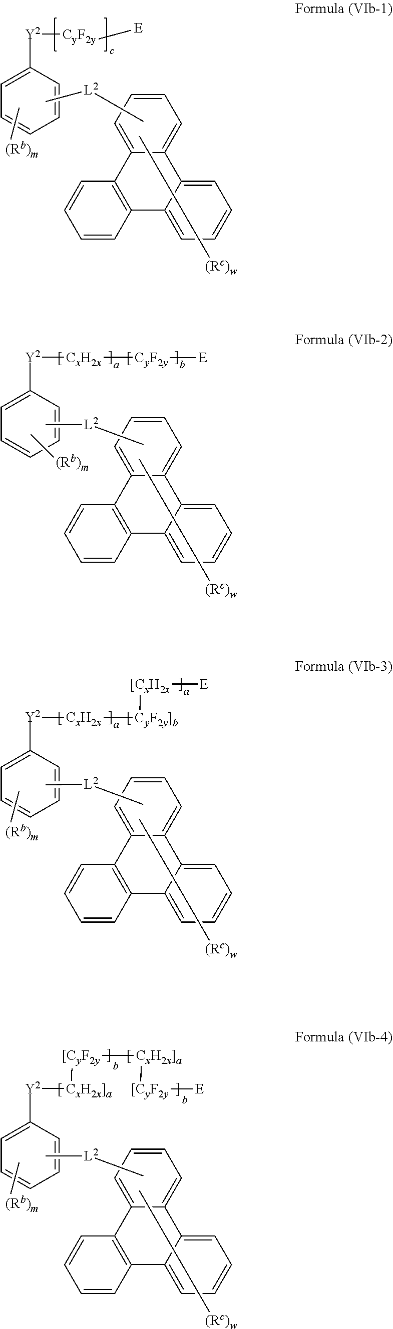

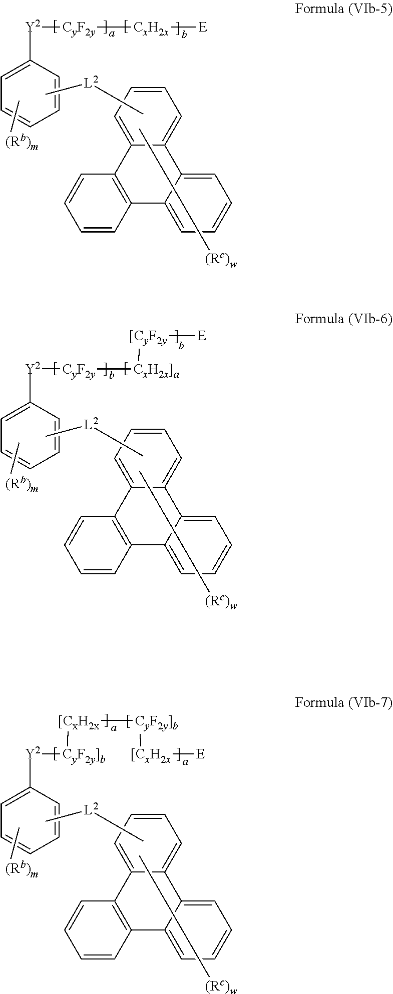

where the symbols L², R^{b} and R^{c} have the definitions given above, especially for formula (I), the symbols Y² and E and the indices a, b, c, x and y have the definitions given above, especially for formulae (I-1) to (I-7), the index w is 0, 1, 2, 3, 4, 5, 6, 7, 8, 9, 10 or 11, preferably 0, 1, 2, 3 or 4, more preferably 0, 1 or 2; and the index m is 0, 1, 2, 3 or 4, preferably 0, 1 or 2, more preferably 0 or 1.

In a further-preferred configuration, it may be the case that the compounds of the invention comprise a structure of the formulae (VIc-1) to (VIc-7), where the compounds of the invention may more preferably be selected from the compounds of the formulae (VIc-1) to (VIc-7),

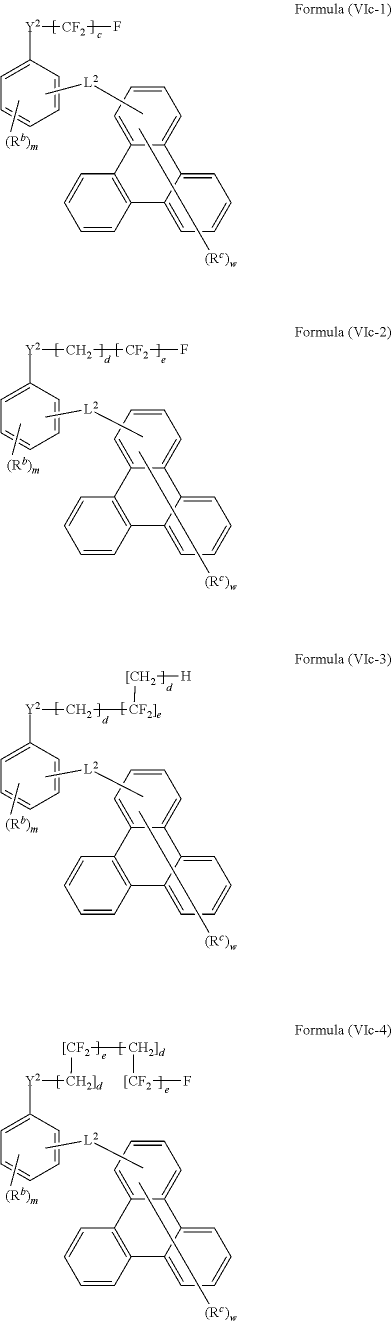

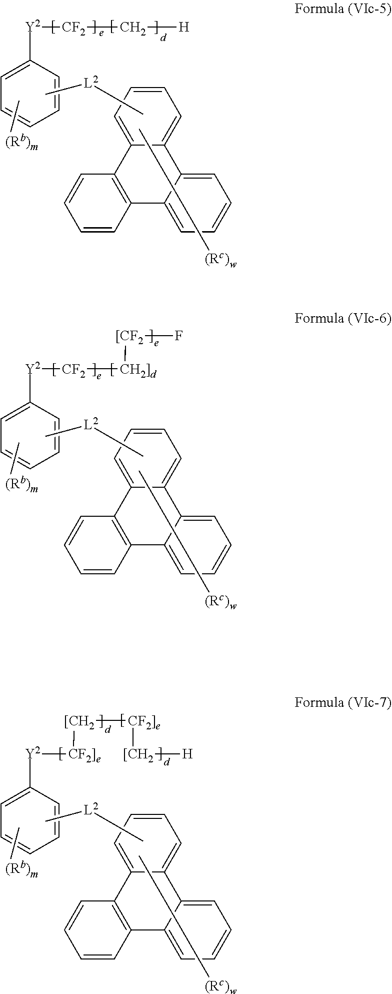

where the symbols L², R^{b} and R^{c} have the definitions given above, especially for formula (I), the symbol Y² and the index c have the definitions given above, especially for formulae (I-1) to (I-7), the indices d and e have the definitions given above, especially for formulae (Ia-1) to (Ia-7), the index w is 0, 1, 2, 3, 4, 5, 6, 7, 8, 9, 10 or 11, preferably 0, 1, 2, 3 or 4, more preferably 0, 1 or 2; and the index m is 0, 1, 2, 3 or 4, preferably 0, 1 or 2, more preferably 0 or 1.

Preferably, two R^{b} radicals together with the heteroaromatic or aromatic groups to which these R^{b} radicals bind do not form a fused aromatic or heteroaromatic ring system, this including possible substituents R¹, R² by which the R^{b} radicals may be substituted. This is applicable especially to the structures of the formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7), and the further preferred configurations of these structures and compounds that are described above and hereinafter.

In addition, in formulae (I), (II), (III), (IV), (V) and/or (VI) inter alia, it may be the case that the numerical ratio of fluorine atoms to carbon atoms in the FA′ group is at least 0.5, preferably at least 0.75 and more preferably at least 1.

It may preferably be the case, in formulae (I), (II), (III), (IV), (V) and/or (VI) inter alia, that the numerical ratio of hydrogen atoms to fluorine atoms in the FA′ group is not more than 1, preferably not more than 0.75 and more preferably not more than 0.5, where the FA′ group more preferably comprises not more than 10, preferably not more than 6, more preferably not more than 4 and especially preferably no hydrogen atoms.

It may additionally be the case, in formulae (I), (II), (III), (IV), (V) and/or (VI) inter alia, that the FA′ group comprises not more than 20, preferably not more than 16, more preferably not more than 12 and especially preferably not more than 10 carbon atoms.

In a particularly preferred configuration of the present invention, for example in formulae (I-1) to (I-7), (Ib-1) to (Ib-7), (II-1) to (II-7), (IIb-1) to (IIb-7), (III-1) to (III-7), (IIIb-1) to (IIIb-7), (IV-1) to (IV-7), (IVb-1) to (IVb-7), (V-1) to (V-7), (Vb-1) to (Vb-7), (VI-1) to (VI-7) and (VIb-1) to (VIb-7), it may be the case that the ratio of the indices a and b (a/b) is in the range from 4:1 to 1:8, preferably 2:1 to 1:4 and more preferably 1:1 to 1:3.

It may preferably be the case that the sum total of the indices a and x is not more than 10, preferably not more than 7 and more preferably not more than 5, and that of the indices b and y is not more than 10, preferably not more than 7 and more preferably not more than 5. This preference is applicable especially to structures of the formulae (I-1) to (I-7), (Ib-1) to (Ib-7), (II-1) to (II-7), (IIb-1) to (IIb-7), (III-1) to (III-7), (IIIb-1) to (IIIb-7), (IV-1) to (IV-7), (IVb-1) to (IVb-7), (V-1) to (V-7), (Vb-1) to (Vb-7), (VI-1) to (VI-7) and (VIb-1) to (VIb-7), and the further preferred configurations of these structures and compounds that are described above and hereinafter.

In a particularly preferred configuration of the present invention, for example in formulae (Ia-1) to (Ia-7), (Ic-1) to (Ic-7), (IIa-1) to (IIa-7), (IIc-1) to (IIc-7), (IIIa-1) to (IIIa-7), (IIIc-1) to (IIIc-7), (IVa-1) to (IVa-7), (IVc-1) to (IVc-7), (Va-1) to (Va-7), (Vc-1) to (Vc-7), (VIa-1) to (VIa-7) and (VIc-1) to (VIc-7), it may be the case that the ratio of the indices d and e (d/e) is in the range from 4:1 to 1:8, preferably 2:1 to 1:4 and more preferably 1:1 to 1:3.

Preferably, for example in formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7), it may be the case that these structures have a numerical ratio of fluorine atoms to carbon atoms of at least 0.5, preferably at least 0.75 and more preferably at least 1.

In addition, for example in formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7), it may be the case that at least one, preferably at least two, of the substituent(s) R^{b} is/are selected from F or a fluorinated alkyl radical having 1 to 20 carbon atoms.

If a radical, for example an R, R^{a}, R^{b}, R^{c}, R¹, R², R³ and/or R⁴ radical, includes or represents a fluorinated alkyl radical having 1 to 20 carbon atoms, it may preferably be the case that the fluorinated alkyl radical having 1 to 20 carbon atoms has a numerical ratio of hydrogen atoms to fluorine atoms of not more than 1, preferably not more than 0.75 and more preferably not more than 0.5, where the fluorinated alkyl radical having 1 to 20 carbon atoms more preferably has not more than 10, preferably not more than 6, more preferably not more than 4, hydrogen atoms, and especially preferably comprises none.

A compound usable in accordance with the invention may have a connecting group, which is set out in detail, for example, in structures (Ar-1) to (Ar-18) and/or (Ar′-1) to (Ar′-18) as the L¹ radical. In addition, the structures of the formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7) comprise connecting groups L².

In a further preferred embodiment of the invention, L¹, L² is a bond or an aromatic or heteroaromatic ring system which has 5 to 14 aromatic or heteroaromatic ring atoms, preferably an aromatic ring system which has 6 to 12 carbon atoms, and which may be substituted by one or more R¹ radicals, but is preferably unsubstituted, where R¹ may have the definition given above, especially for formula (SE-I), (SE-II) and/or (SE-III). More preferably, L¹, L² is an aromatic ring system having 6 to 10 aromatic ring atoms or a heteroaromatic ring system having 6 to 13 heteroaromatic ring atoms, each of which may be substituted by one or more R² radicals, but is preferably unsubstituted, where R² may have the definition given above, especially for formula (SE-I), (SE-II) and/or (SE-III).

Further preferably, the symbol L¹ set out in formulae (Ar-1) to (Ar-18) and/or (Ar′-1) to (Ar′-18) inter alia or the symbol L² set out in formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded directly, i.e. via an atom of the aromatic or heteroaromatic group, to the respective atom of the further group.

It may additionally be the case that the L¹ or L² group comprises an aromatic ring system having not more than two fused aromatic and/or heteroaromatic 6-membered rings, and preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L¹, L² are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R¹ radicals, but are preferably unsubstituted.

It may further be the case that the L¹ or L² group has not more than 1 nitrogen atom, preferably not more than 2 heteroatoms, especially preferably not more than one heteroatom and more preferably no heteroatom.

In a preferred embodiment, it may be the case that the compound comprises at least one connecting group selected from the formulae (L¹-1) to (L¹-74), or the L¹ radical in formulae (Ar-1) to (Ar-18) and/or (Ar′-1) to (Ar′-18) represents a bond or is a group selected from the formulae (L¹-1) to (L¹-74), or the L² radical in formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7) represents a bond or is a group selected from the formulae (L¹-1) to (L¹-74),

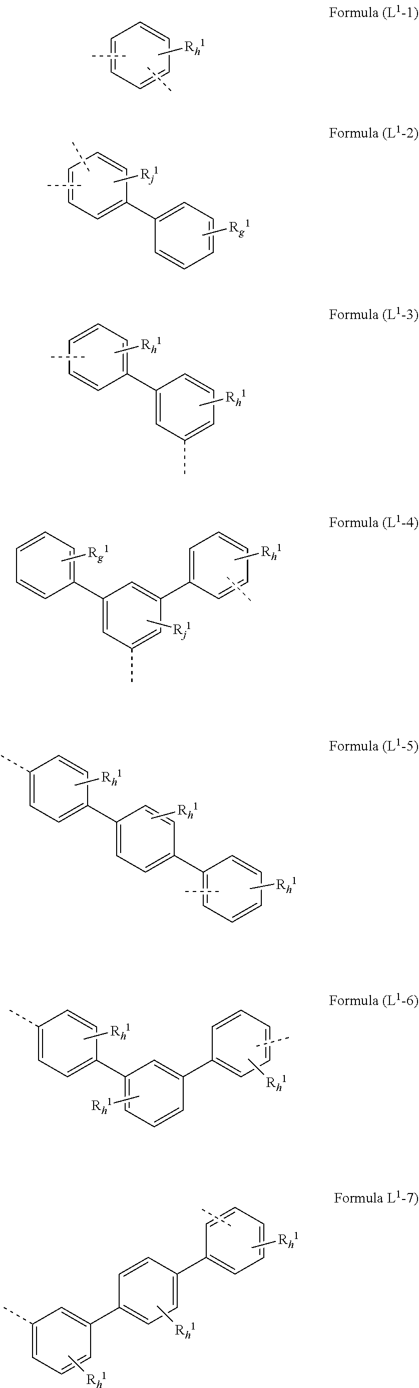

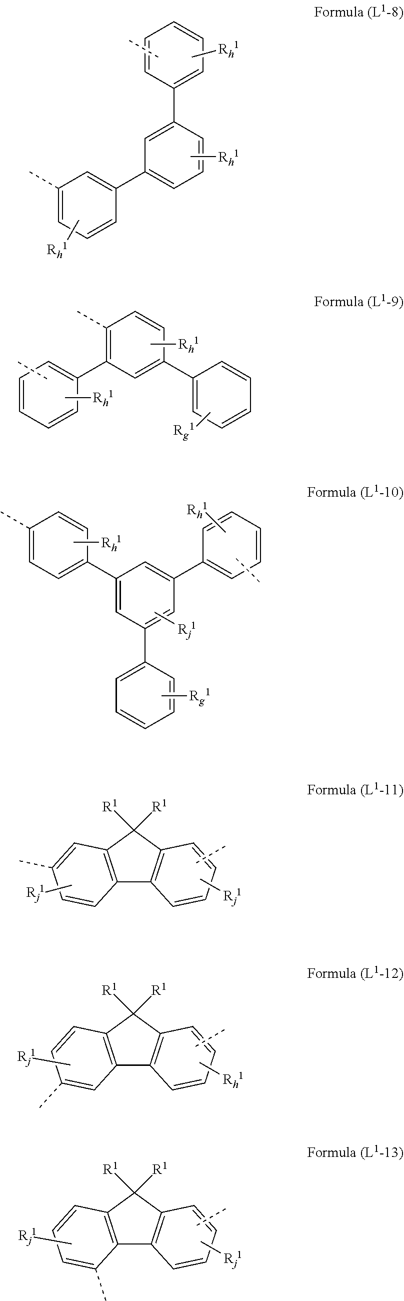

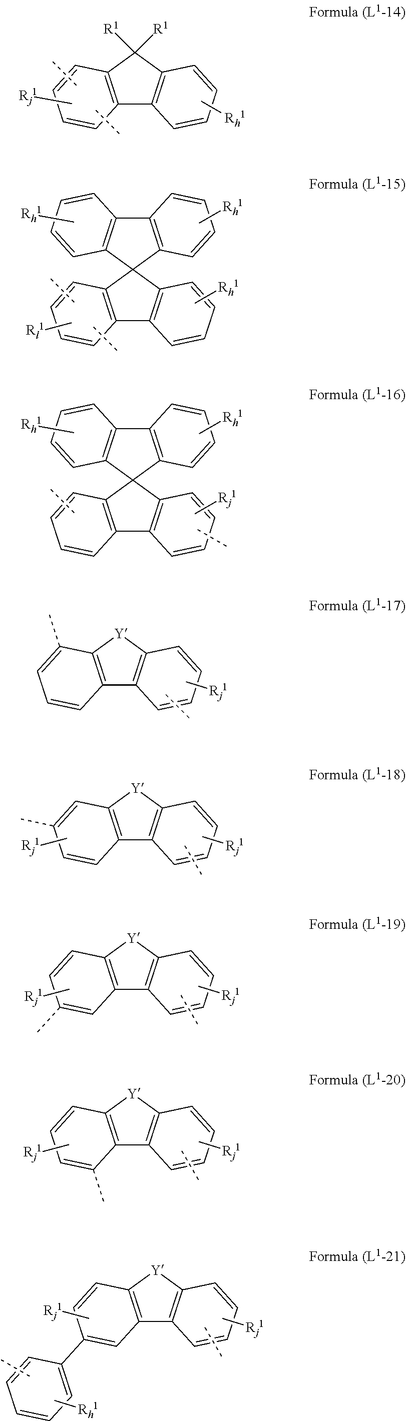

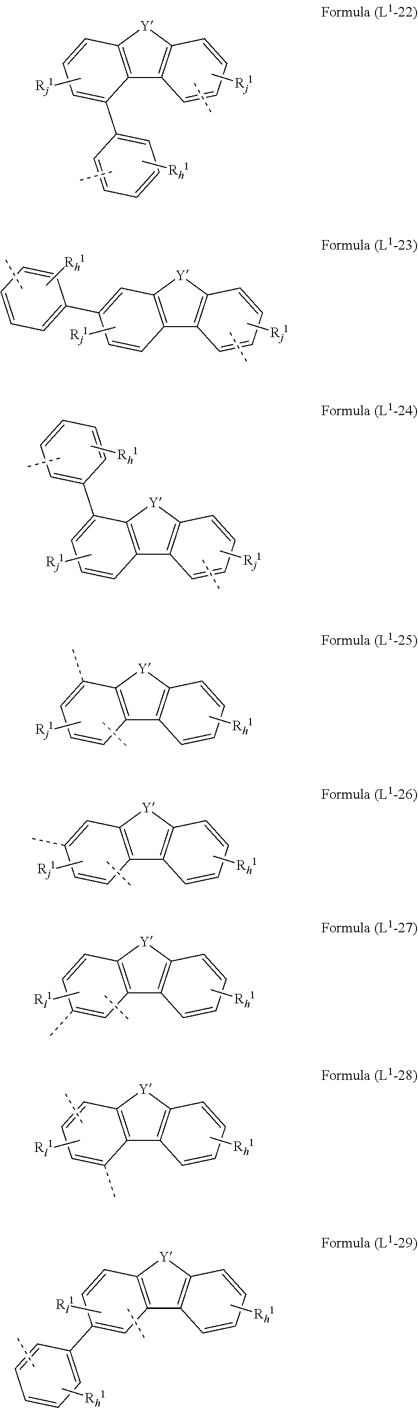

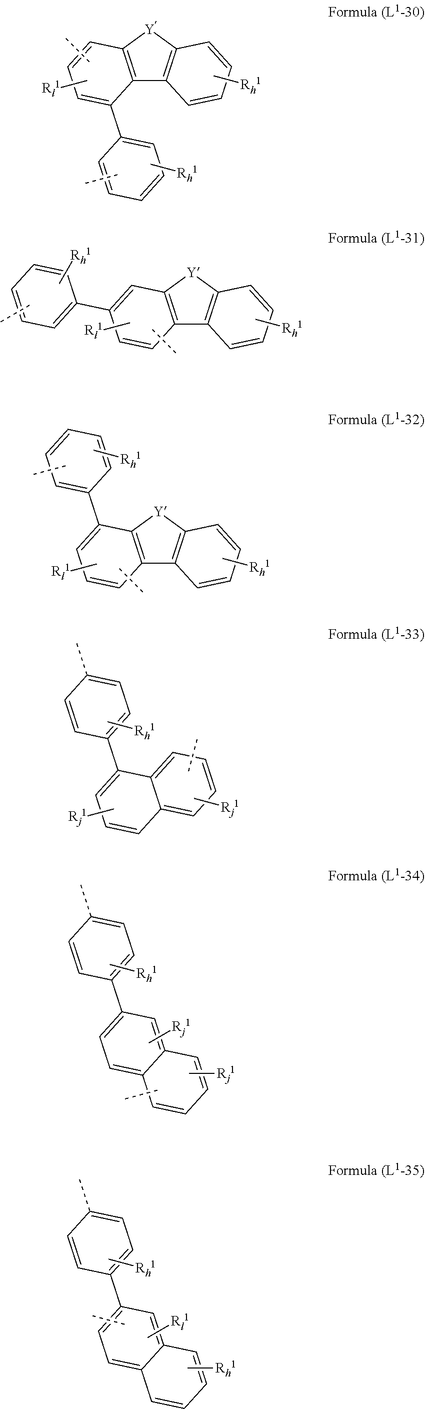

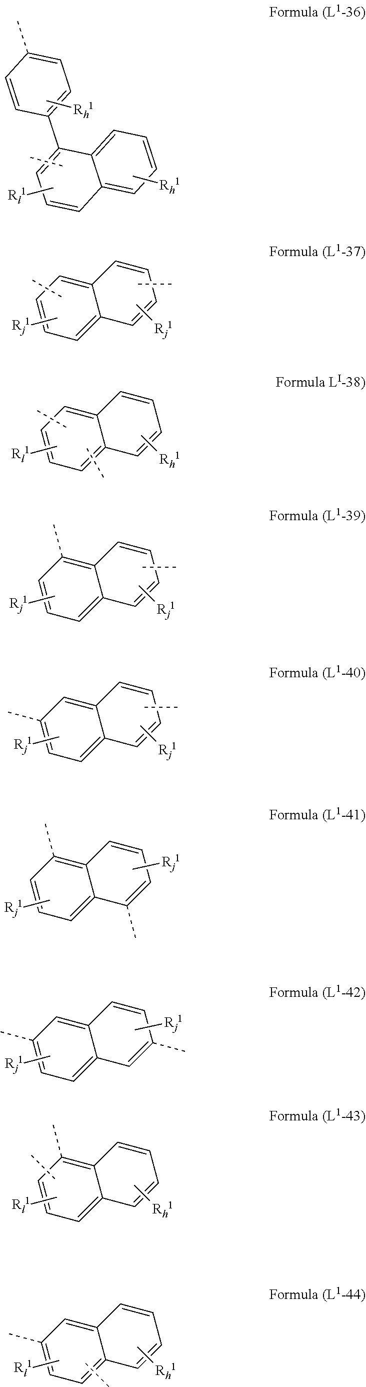

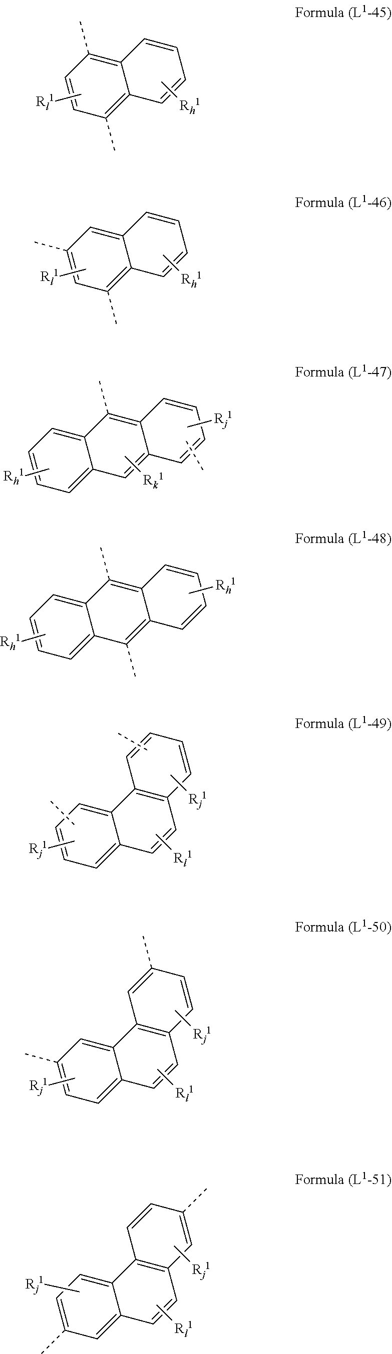

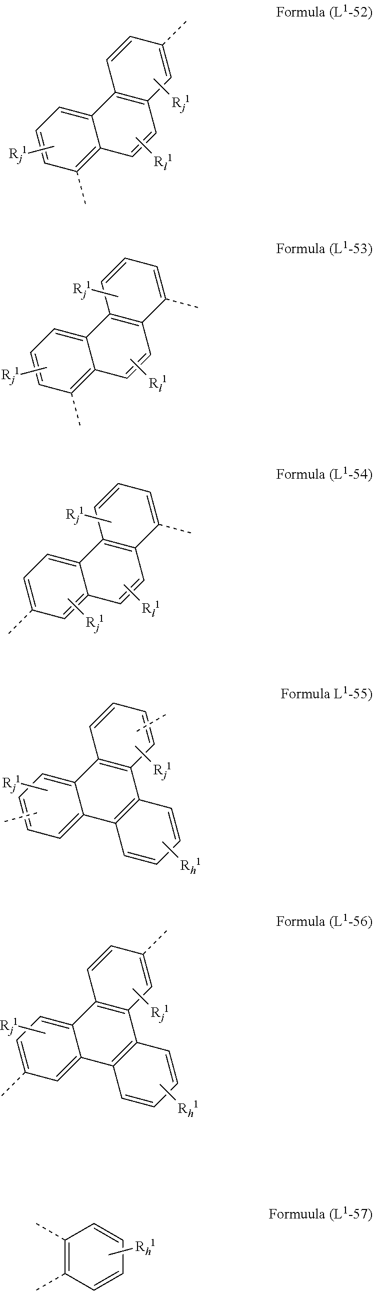

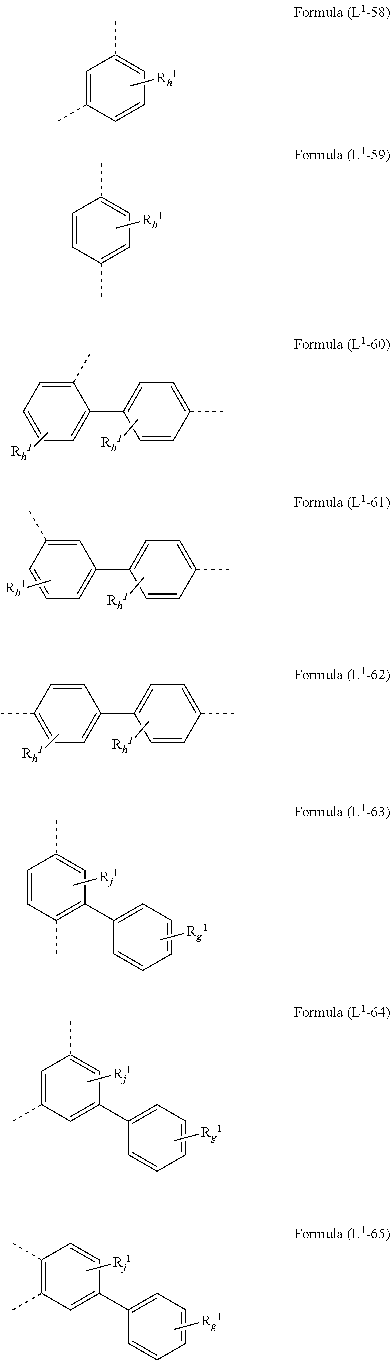

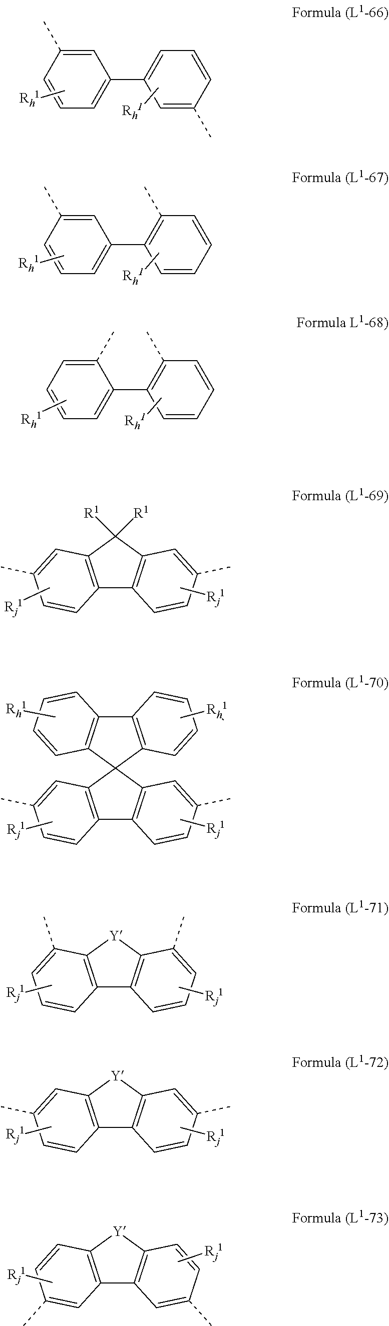

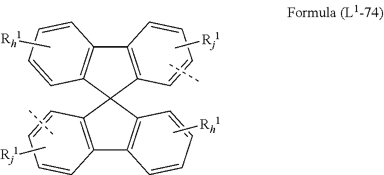

where the dotted bonds in each case mark the positions of attachment, the index k is 0 or 1, the index l is 0, 1 or 2, the index j at each instance is independently 0, 1, 2 or 3, the index h at each instance is independently 0, 1, 2, 3 or 4, the index g is 0, 1, 2, 3, 4 or 5; the symbol Y′ is O, S, BR¹ or NR¹, preferably O or NR¹; and the symbol R¹ has the definition given above, especially for formula (SE-I), (SE-II) and/or (SE-III).

The sum total of the indices k, l, g, h and j in the structures of the formula (L¹-1) to (L¹-74) is preferably not more than 3 in each case, preferably not more than 2 and especially preferably not more than 1.

Preferred compounds having a group of the formulae (Ar-1) to (Ar-18) and/or (Ar′-1) to (Ar′-18) comprise an L¹ group selected from a bond or one of the formulae (L¹-1) to (L¹-46) and/or (L¹-57) to (L¹-74), preferably of the formula (L¹-1) to (L¹-32) and/or (L¹-57) to (L¹-74), especially preferably of the formula (L¹-1) to (L¹-10) and/or (L¹-57) to (L¹-68). Advantageously, the sum total of the indices k, l, g, h and j in the structures of the formulae (L¹-1) to (L¹-46) and/or (L¹-57) to (L¹-74), preferably of the formula (L¹-1) to (L¹-32) and/or (L¹-57) to (L¹-74), especially preferably of the formula (L¹-1) to (L¹-10) and/or (L¹-57) to (L¹-68), may in each case be not more than 3, preferably not more than 2 and more preferably not more than 1.

Preferred compounds having a structure of the formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (111a-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7) comprise a L² group selected from a bond or one of the formulae (L¹-1) to (L¹-46) and/or (L¹-57) to (L¹-74), preferably of the formulae (L¹-1) to (L¹-32) and/or (L¹-57) to (L¹-74), especially preferably of the formula (L¹-1) to (L¹-10) and/or (L¹-57) to (L¹-68). Advantageously, the sum total of the indices k, l, g, h and j in the structures of the formulae (L¹-1) to (L¹-46) and/or (L¹-57) to (L¹-74), preferably of the formula (L¹-1) to (L¹-32) and/or (L¹-57) to (L¹-74), especially preferably of the formula (L¹-1) to (L¹-10) and/or (L¹-57) to (L¹-68), may in each case be not more than 3, preferably not more than 2 and more preferably not more than 1.

When two radicals that may especially be selected from R, R^{a}, R^{b}, R^{c}, R¹, R², R³ and/or R⁴ form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another. In addition, the ring systems provided with the substituents R, R^{a}, R^{b}, R^{c}, R¹, R², R³ and/or R⁴ may also be joined to one another via a bond, such that this can bring about a ring closure. In this case, each of the corresponding bonding sites has preferably been provided with a substituent R, R^{a}, R^{b}, R^{c}, R¹, R², R³ and/or R⁴.

It may preferably be the case that the substituents R, R^{a}, R^{b}, R^{c}, R¹, R², R³ and/or R⁴ of the structures set out above and hereinafter do not form any fused aromatic or heteroaromatic ring system, preferably any fused ring system. This includes the formation of a fused ring system with possible substituents R¹ and R² that may be bonded to the R^{a}, R^{b}, R^{c} and/or R or to R¹ radicals.

It may preferably be the case that at least one of the R, R^{a}, R^{b} and/or R^{c} radicals is selected from the group of the phenyls, fluorenes, indenofluorenes, spirobifluorenes, carbazoles, indenocarbazoles, indolocarbazoles, spirocarbazoles, pyrimidines, triazines, quinazolines, quinoxalines, pyridines, quinolines, isoquinolines, lactams, triarylamines, dibenzofurans, dibenzothienes, imidazoles, benzimidazoles, benzoxazoles, benzothiazoles, 5-arylphenanthridin-6-ones, 9,10-dehydrophenanthrenes, fluoranthenes, naphthalenes, phenanthrenes, anthracenes, benzanthracenes, fluoradenes, pyrenes, perylenes, chrysenes, borazines, boroxines, boroles, borazoles, azaboroles, ketones, phosphine oxides, arylsilanes, siloxanes and combinations thereof.

It may further be the case that at least one of the R, R^{a}, R^{b} and/or R^{c} radicals is selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 9,9′-diarylfluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3-, 4- or 9-carbazolyl, 1- or 2-naphthyl, anthracenyl, preferably 9-anthracenyl, trans- and cis-indenofluorenyl, indenocarbazolyl, indolocarbazolyl, spirocarbazolyl, 5-arylphenanthridin-6-onyl, 9,10-dehydrophenanthrenyl, fluoranthenyl, tolyl, mesityl, phenoxytolyl, anisolyl, triarylaminyl, bis(triarylaminyl), tris(triarylaminyl), hexamethylindanyl, tetralinyl, monocycloalkyl, biscycloalkyl, tricycloalkyl, alkyl, for example tert-butyl, methyl, propyl, alkoxy, alkylsulfanyl, alkylaryl, triarylsilyl, trialkylsilyl, xanthenyl, 10-arylphenoxazinyl, phenanthrenyl and/or triphenylenyl, each of which may be substituted by one or more radicals, but are preferably unsubstituted, particular preference being given to phenyl, spirobifluorene, fluorene, dibenzofuran, dibenzothiophene, anthracene, phenanthrene, triphenylene groups.

Preferred aromatic or heteroaromatic ring systems R, R^{a}, R^{b}, R^{c}, Ar′ and/or Ar are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3, 4 or 9 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹ or R radicals.

There follows a description of preferred substituents R, R^{a}, R^{b} and R^{c}. In a preferred embodiment of the invention, R, R^{a}, R^{b}, R^{c} are the same or different at each instance and are selected from the group consisting of H, D, F, CN, NO₂, Si(R¹)₃, B(OR¹)₂, a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R¹ radicals.

In a further-preferred embodiment of the invention, substituent R, R^{a}, R^{b}, R^{c} is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R¹ radicals.

It may further be the case that at least one substituent R, R^{a}, R^{b}, R^{c} is the same or different at each instance and is selected from the group consisting of H, D, an aromatic or heteroaromatic ring system which has 6 to 30 aromatic ring atoms and may be substituted by one or more R¹ radicals, and an N(Ar′)₂ group. In a further-preferred embodiment of the invention, the substituent R, R^{a}, R^{b}, R^{c} is the same or different at each instance and is selected from the group consisting of H, D, an aromatic or heteroaromatic ring system which has 6 to 30 aromatic ring atoms and may be substituted by one or more R¹ radicals, and an N(Ar′)₂ group. More preferably, substituent R, R^{a}, R^{b}, R^{c} is the same or different at each instance and is selected from the group consisting of H or an aromatic or heteroaromatic ring system having 6 to 24 aromatic ring atoms, preferably having 6 to 18 aromatic ring atoms, more preferably having 6 to 13 aromatic ring atoms, each of which may be substituted by one or more R¹ radicals.

Preferred aromatic or heteroaromatic ring systems for the substituent R, R^{a}, R^{b}, R^{c}, R¹, R³, R⁴ or Ar or Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹ or R² radicals. Particular preference is given to the structures R¹-1 to R¹-43 shown below, with particular preference for structures of the formulae R¹-1, R¹-3, R¹-4, R¹-10, R¹-11, R¹-12, R¹-13, R¹-14, R¹-16, R¹-17, R¹-18, R¹-19, R¹-20, R¹-21 and/or R¹-22: With regard to the structures R¹-1 to R¹-43, it should be stated that these are shown with a substituent R². In the case of the ring systems R, R^{a}, R^{b}, R^{c}, these substituents R² should be replaced by R¹.

When the groups set out above are substituted by substituents R¹, R³, R⁴, these substituents R¹, R³, R⁴ are preferably selected from the group consisting of H, D, F, CN, N(Ar″)₂, C(═O) Ar″, P(═O)(Ar″)₂, a straight-chain alkyl or alkoxy group having 1 to 10 carbon atoms or a branched or cyclic alkyl or alkoxy group having 3 to 10 carbon atoms or an alkenyl group having 2 to 10 carbon atoms, each of which may be substituted by one or more R² radicals, where one or more nonadjacent CH₂ groups may be replaced by O and where one or more hydrogen atoms may be replaced by D or F, an aromatic or heteroaromatic ring system which has 5 to 24 aromatic ring atoms and may be substituted in each case by one or more R² radicals, but is preferably unsubstituted, or an aralkyl or heteroaralkyl group which has 5 to 25 aromatic ring atoms and may be substituted by one or more R² radicals; at the same time, it is optionally possible for two substituents R¹, R³, R⁴ preferably bonded to adjacent carbon atoms to form a monocyclic or polycyclic, aliphatic, aromatic or heteroaromatic ring system which may be substituted by one or more R² radicals; where the R² and Ar″ groups have the definitions given above, especially for formula (SE-I), (SE-II) and/or (SE-III).

More preferably, these substituents R¹, R³, R⁴ are selected from the group consisting of H, D, F, CN, N(Ar″)₂, a straight-chain alkyl group having 1 to 8 carbon atoms, preferably having 1, 2, 3 or 4 carbon atoms, or a branched or cyclic alkyl group having 3 to 8 carbon atoms, preferably having 3 or 4 carbon atoms, or an alkenyl group having 2 to 8 carbon atoms, preferably having 2, 3 or 4 carbon atoms, each of which may be substituted by one or more R² radicals, but is preferably unsubstituted, or an aromatic or heteroaromatic ring system which has 5 to 24 aromatic ring atoms, preferably 6 to 18 aromatic ring atoms, more preferably 6 to 13 aromatic ring atoms, and may be substituted in each case by one or more nonaromatic R¹, R³, R⁴ radicals, but is preferably unsubstituted; at the same time, two substituents R¹, R³, R⁴ preferably bonded to adjacent carbon atoms may optionally form a monocyclic or polycyclic aliphatic ring system which may be substituted by one or more R² radicals, but is preferably unsubstituted, where Ar″ may have the definition set out above.

Most preferably, the substituents R¹, R³, R⁴ are selected from the group consisting of H or an aromatic or heteroaromatic ring system which has 6 to 18 aromatic ring atoms, preferably 6 to 13 aromatic ring atoms, and may be substituted in each case by one or more nonaromatic R² radicals, but is preferably unsubstituted. Examples of suitable substituents R¹ are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, 1-, 2-, 3- or 4-carbazolyl and indenocarbazolyl, each of which may be substituted by one or more R² radicals, but are preferably unsubstituted.

It may further be the case that the substituents R¹, R³, R⁴ of a ring system do not form a fused aromatic or heteroaromatic ring system, preferably any fused ring system, with other ring atoms of the ring system. This includes the formation of a fused ring system with possible substituents R² which may be bonded to the R¹, R³, R⁴ radicals.

It may further be the case that, in a structure of formula (SE-I), (SE-II), (SE-III), (SE-I) to (SE-21) and/or (SE-1a) to (SE-21a), at least one R¹ or Ar″ radical is a group selected from the formulae (R¹-1) to (R¹-43), or, in a structure of formula (Ar-1) to (Ar-18) and/or (Ar′-1) to (Ar′-18), at least one R¹ is a group selected from the formulae (R¹-1) to (R¹-43), or, in a structure of formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (11c-7), (III), (III-1) to (III-7), (IIIa-1) to (IIIa-7), (IIIb-1) to (IIIb-7), (IIIc-1) to (111c-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7), at least one R¹ is a group selected from the formulae (R¹-1) to (R¹-43),

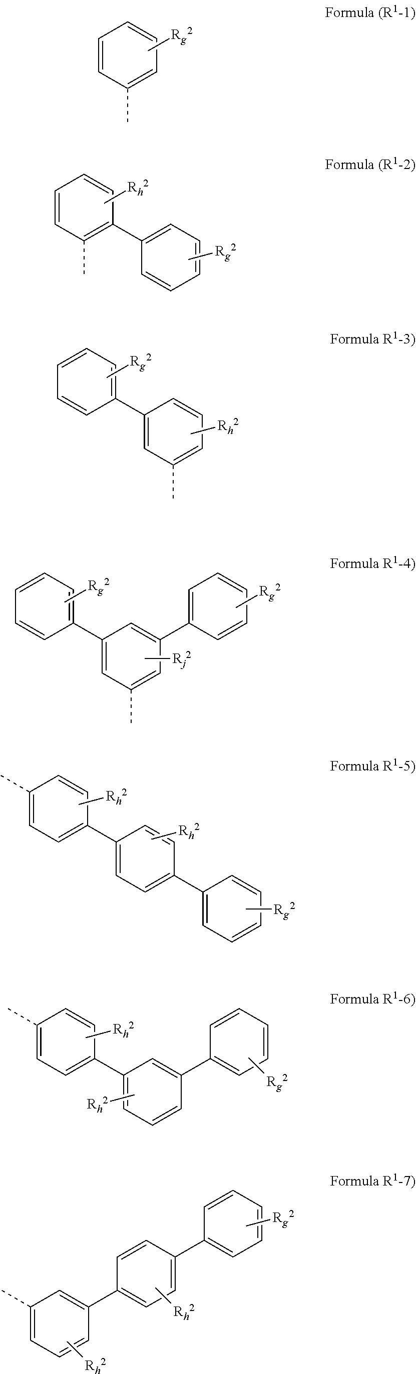

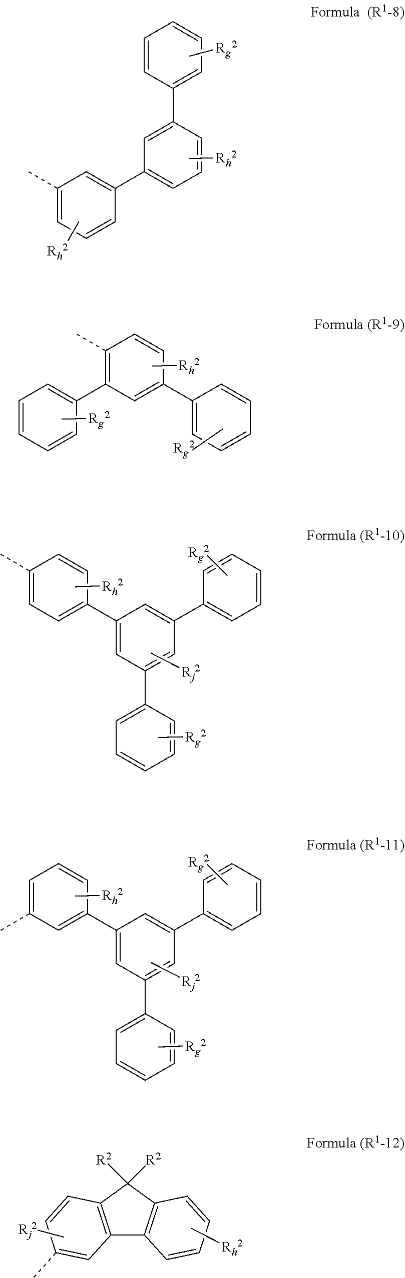

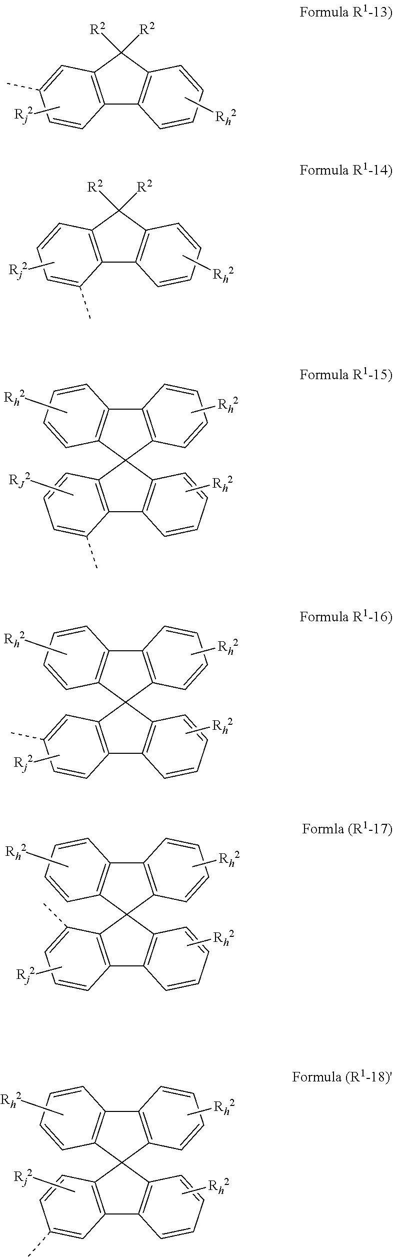

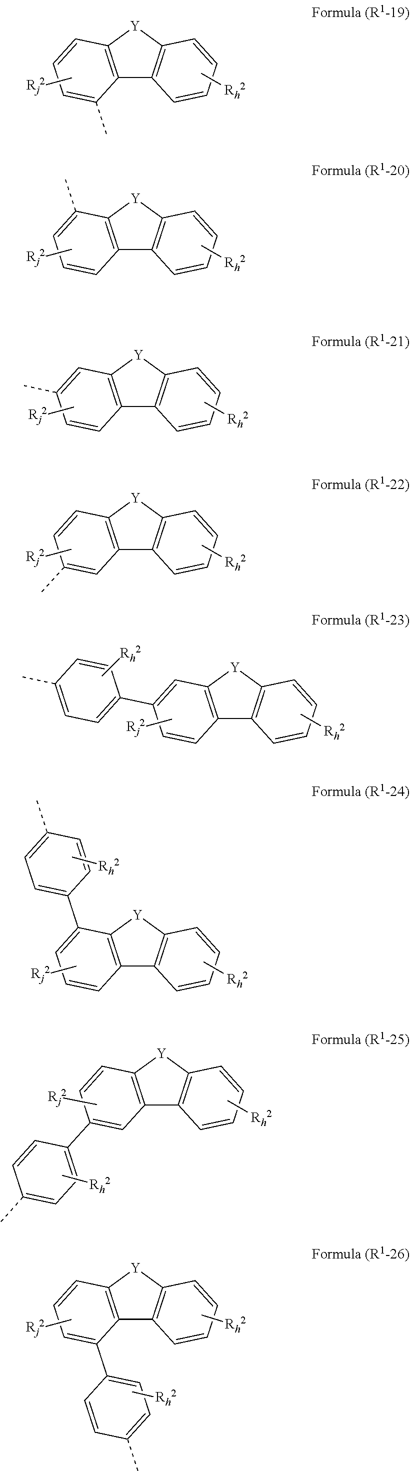

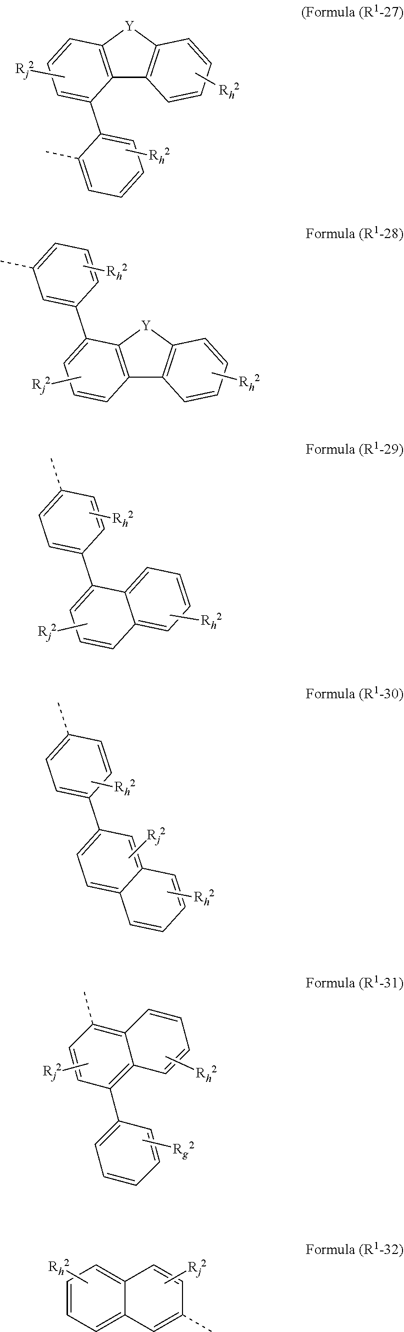

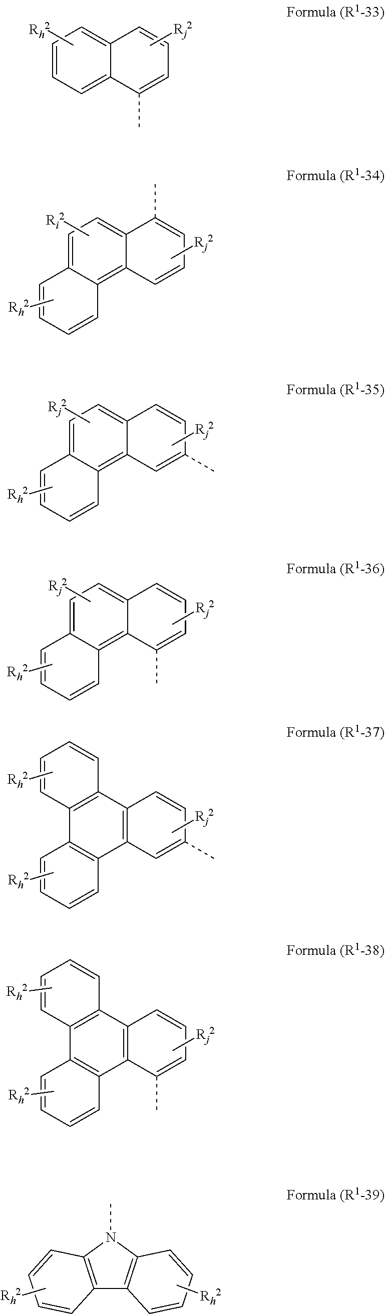

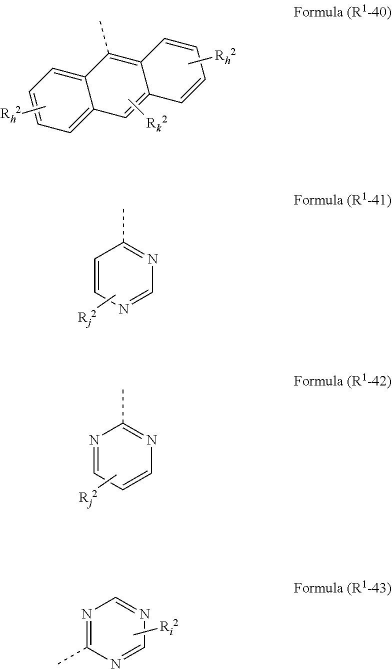

where the symbols used are as follows:
Y is O, S or NR², preferably 0 or 5,
k at each instance is independently 0 or 1;
i at each instance is independently 0, 1 or 2;
j at each instance is independently 0, 1, 2 or 3;
h at each instance is independently 0, 1, 2, 3 or 4;
g at each instance is independently 0, 1, 2, 3, 4 or 5;
R² has the definition given above, especially for formula (SE-I), (SE-II)
and/or (SE-III), and the dotted bond marks the position of attachment.

It may preferably be the case that the sum total of the indices k, i, j, h and g in the structures of the formula (R¹-1) to (R¹-43) in each case is not more than 3, preferably not more than 2 and more preferably not more than 1.

In a further preferred embodiment of the invention, R¹, R³, R⁴ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R² radicals. In a particularly preferred embodiment of the invention, R¹, R³, R⁴ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 13 aromatic ring atoms and may be substituted in each case by one or more R² radicals, but is preferably unsubstituted.

In a further preferred embodiment of the invention, R² is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

Preferred compounds usable in accordance with the invention and/or compounds of the invention preferably have a sublimation temperature which is preferably within a range of 150 to 400° C., more preferably in the range from 180 to 360° C. and especially preferably in the range from 220 to 340° C., measured to DIN 51006. The sublimation temperature is found here from the vacuum TGA measurement in which a material is sublimed or evaporated in a controlled manner. The measurement can be conducted with a TG 209 F1 Libra instrument from Netzsch with the following measurement conditions:
Sample weight: 1 mg
Crucible: open aluminium crucible
Heating rate: 5 K/min
Temperature range: 105-550° C.
Atmosphere: Vacuum 10-2 mbar (regulated)
Evacuation time before commencement of measurement: about 30 minutes. The sublimation temperature used is the temperature at which 5% weight loss occurs.

It may further be the case that the compound comprises at least two, preferably at least three, four or more, more preferably exactly two or exactly three, structuring elements according to the above-defined formula (SE-I), (SE-II), (SE-III) and/or at least two, preferably at least three, four or more, more preferably exactly two or exactly three, structures according to the above-defined formula (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7).

In a preferred configuration, a compound of the invention can be represented by at least one of the structures of formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (111a-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7). Preferably, compounds of the invention, preferably comprising structures of formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and/or (VIc-1) to (VIc-7), have a molecular weight of not more than 5000 g/mol, preferably not more than 4000 g/mol, particularly preferably not more than 3000 g/mol, especially preferably not more than 2000 g/mol and most preferably not more than 1200 g/mol.

In addition, it is a feature of preferred compounds of the invention that they are sublimable. These compounds generally have a molar mass of less than about 1200 g/mol.

The abovementioned preferred embodiments may be combined with one another as desired within the restrictions defined in claim 1. In a particularly preferred embodiment of the invention, the abovementioned preferences occur simultaneously.

Preferred embodiments of compounds of the invention are recited in detail in the examples, these compounds being usable alone or in combination with further compounds for all purposes of the invention.

Provided that the conditions essential to the invention are met, the abovementioned preferred embodiments can be combined with one another as desired. In a particularly preferred embodiment of the invention, the abovementioned preferred embodiments apply simultaneously.

The compounds usable in accordance with the invention and the novel compounds of the invention are preparable in principle by various methods. However, the processes described hereinafter have been found to be particularly suitable.

Therefore, the present invention further provides a process for preparing the compounds of the invention, in which, in a coupling reaction, a compound comprising at least one fluorinated alkyl radical having at least two carbon atoms is joined to a compound comprising at least one aromatic or heteroaromatic group.

Suitable compounds comprising at least one fluorinated alkyl radical having at least two carbon atoms are in many cases commercially available, with the starting compounds detailed in the examples being obtainable by known processes, and so reference is made thereto.

These compounds can be reacted with further compounds by known coupling reactions, the necessary conditions for this purpose being known to the person skilled in the art, and detailed specifications in the examples giving support to the person skilled in the art in conducting these reactions.

Particularly suitable and preferred coupling reactions which all lead to C—C bond formations and/or C—N bond formations are those according to BUCHWALD, SUZUKI, YAMAMOTO, STILLE, HECK, NEGISHI, SONOGASHIRA and HIYAMA. These reactions are widely known, and the examples will provide the person skilled in the art with further pointers.

The principles of the preparation processes detailed above are known in principle from the literature for similar compounds and can be adapted easily by the person skilled in the art for the preparation of the compounds of the invention. Further information can be found in the examples.

Particularly suitable compounds can be obtained with the following aryl bromides, listed by CAS number, with the boronic esters S: S1, S2, S3, S4, S5, S6, S7, S8, S9, S10, S11, S12, S13, S14, S15, S16, S17, S18, S9, S20, S21, S22, S23, S24, where the boronic esters S: S1, S2, S3, S4, S5, S6, S7, S8, S9, S10, S11, S12, S13, S14, S15, S16, S17, S18, S9, S20, S21, S22, S23, S24 are detailed in the examples. The compounds of the invention are prepared in yields of about 50-90%, where the regiochemistry of the C—C coupling is unambiguously fixed by the position of the aryl bromide and arylboronic acid coupling partners. If the aryl bromides are di-, tri-, tetrabromides etc., the stoichiometry is adjusted correspondingly such that all Br functions react under C—C coupling:

| | | |
|---|---|---|
| Boronic ester | Aryl bromide | Products |

Bromonaphthalenes

| | | |
|---|---|---|
| S1 to S24 | 90-11-9 | [90-11-9]-S1 to S24 |
| S1 to S24 | 37621-57-1 | [37621-57-1]-S1 to S24 |
| S1 to S24 | 1838583-18-8 | [1838583-18-8]-S1 to S24 |
| S1 to S24 | 59951-65-4 | [59951-65-4]-S1 to S24 |
| S1 to S24 | 911836-38-9 | [911836-38-9]-S1 to S24 |
| S1 to S24 | 1637445-80-7 | [1637445-80-7]-S1 to S24 |
| S1 to S24 | 1822374-60-6 | [1822374-60-6]-S1 to S24 |
| S1 to S24 | 2121574-80-7 | [2121574-80-7]-S1 to S24 |
| S1 to S24 | 897671-73-7 | [897671-73-7]-S1 to S24 |
| S1 to S24 | 1622010-82-5 | [1622010-82-5]-S1 to S24 |
| S1 to S24 | 1233141-64-4 | [1233141-64-4]-S1 to S24 |
| S1 to S24 | 2096506-54-4 | [2096506-54-4]-S1 to S24 |
| S1 to S24 | 2326502-69-4 | [2326502-69-4]-S1 to S24 |
| S1 to S24 | 2421137-98-4 | [2421137-98-4]-S1 to S24 |
| S1 to S24 | 1220509-64-7 | [1220509-64-7]-S1 to S24 |
| S1 to S24 | 2112854-24-5 | [2112854-24-5]-S1 to S24 |
| S1 to S24 | 1158227-46-3 | [1158227-46-3]-S1 to S24 |
| S1 to S24 | 2189205-14-6 | [2189205-14-6]-S1 to S24 |
| S1 to S24 | 1610364-54-9 | [1610364-54-9]-S1 to S24 |
| S1 to S24 | 19997-07-0 | [19997-07-0]-S1 to S24 |
| S1 to S24 | 1357178-46-1 | [1357178-46-1]-S1 to S24 |
| S1 to S24 | 1326240-05-4 | [1326240-05-4]-S1 to S24 |
| S1 to S24 | 1427062-03-0 | [1427062-03-0]-S1 to S24 |
| S1 to S24 | 1062556-32-4 | [1062556-32-4]-S1 to S24 |
| S1 to S24 | 1314040-11-3 | [1314040-11-3]-S1 to S24 |
| S1 to S24 | 2176471-19-3 | [2176471-19-3]-S1 to S24 |
| S1 to S24 | 2107993-61-1 | [2107993-61-1]-S1 to S24 |
| S1 to S24 | 1448787-71-0 | [1448787-71-0]-S1 to S24 |
| S1 to S24 | 380630-31-9 | [380630-31-9]-S1 to S24 |
| S1 to S24 | 936854-67-0 | [936854-67-0]-S1 to S24 |
| S1 to S24 | 2107993-47-3 | [2107993-47-3]-S1 to S24 |
| S1 to S24 | 1247085-35-3 | [1247085-35-3]-S1 to S24 |
| S1 to S24 | 1207378-69-3 | [1207378-69-3]-S1 to S24 |
| S1 to S24 | 1799604-89-9 | [1799604-89-9]-S1 to S24 |
| S1 to S24 | 1708125-77-2 | [1708125-77-2]-S1 to S24 |
| S1 to S24 | 1207337-63-0 | [1207337-63-0]-S1 to S24 |
| S1 to S24 | 2172969-33-2 | [2172969-33-2]-S1 to S24 |
| S1 to S24 | 1508273-12-8 | [1508273-12-8]-S1 to S24 |
| S1 to S24 | 1508273-17-3 | [1508273-17-3]-S1 to S24 |
| S1 to S24 | 380630-23-9 | [380630-23-9]-S1 to S24 |
| S1 to S24 | 1442439-06-6 | [1442439-06-6]-S1 to S24 |
| S1 to S24 | 2251815-62-8 | [2251815-62-8]-S1 to S24 |
| S1 to S24 | 1637445-89-6 | [1637445-89-6]-S1 to S24 |
| S1 to S24 | 2254697-66-8 | [2254697-66-8]-S1 to S24 |
| S1 to S24 | 1607017-05-9 | [1607017-05-9]-S1 to S24 |
| S1 to S24 | 1443330-46-8 | [1443330-46-8]-S1 to S24 |
| S1 to S24 | 1400556-26-4 | [1400556-26-4]-S1 to S24 |
| S1 to S24 | 1846602-68-3 | [1846602-68-3]-S1 to S24 |
| S1 to S24 | 1820789-10-3 | [1820789-10-3]-S1 to S24 |
| S1 to S24 | 2361069-49-8 | [2361069-49-8]-S1 to S24 |
| S1 to S24 | 2055375-04-5 | [2055375-04-5]-S1 to S24 |
| S1 to S24 | 1647103-06-7 | [1647103-06-7]-S1 to S24 |
| S1 to S24 | 1427316-88-8 | [1427316-88-8]-S1 to S24 |
| S1 to S24 | 1647103-06-7 | [1647103-06-7]-S1 to S24 |
| S1 to S24 | 1544651-04-8 | [1544651-04-8]-S1 to S24 |
| S1 to S24 | 49610-35-7 | [49610-35-7]-S1 to S24 |

Bromoanthracenes

| | | |
|---|---|---|
| S1 to S24 | 1564-64-3 | [1564-64-3]-S1 to S24 |
| S1 to S24 | 23674-17-1 | [23674-17-1]-S1 to S24 |
| S1 to S24 | 23674-20-6 | [23674-20-6]-S1 to S24 |
| S1 to S24 | 1185333-14-5 | [1185333-14-5]-S1 to S24 |
| S1 to S24 | 1420692-37-0 | [1420692-37-0]-S1 to S24 |
| S1 to S24 | 1314653-61-6 | [1314653-61-6]-S1 to S24 |
| S1 to S24 | 1450898-43-7 | [1450898-43-7]-S1 to S24 |
| S1 to S24 | 400607-04-7 | [400607-04-7]-S1 to S24 |
| S1 to S24 | 474688-73-8 | [474688-73-8]-S1 to S24 |
| S1 to S24 | 1263295-26-6 | [1263295-26-6]-S1 to S24 |
| S1 to S24 | 1383720-14-6 | [1383720-14-6]-S1 to S24 |
| S1 to S24 | 2112854-18-7 | [2112854-18-7]-S1 to S24 |
| S1 to S24 | 2271144-14-8 | [2271144-14-8]-S1 to S24 |
| S1 to S24 | 2412830-46-5 | [2412830-46-5]-S1 to S24 |
| S1 to S24 | 1430055-58-5 | [1430055-58-5]-S1 to S24 |
| S1 to S24 | 1539315-06-4 | [1539315-06-4]-S1 to S24 |
| S1 to S24 | 1680182-91-5 | [1680182-91-5]-S1 to S24 |
| S1 to S24 | 1314563-44-4 | [1314563-44-4]-S1 to S24 |
| S1 to S24 | 400607-01-4 | [400607-01-4]-S1 to S24 |
| S1 to S24 | 351464-66-9 | [351464-66-9]-S1 to S24 |
| S1 to S24 | 2098774-28-6 | [2098774-28-6]-S1 to S24 |
| S1 to S24 | 73275-00-0 | [73275-00-0]-S1 to S24 |
| S1 to S24 | 400607-05-8 | [400607-05-8]-S1 to S24 |
| S1 to S24 | 400607-16-1 | [400607-16-1]-S1 to S24 |
| S1 to S24 | 844679-02-3 | [844679-02-3]-S1 to S24 |
| S1 to S24 | 1357911-99-9 | [1357911-99-9]-S1 to S24 |
| S1 to S24 | 1359950-74-5 | [1359950-74-5]-S1 to S24 |
| S1 to S24 | 1421232-43-0 | [1421232-43-0]-S1 to S24 |
| S1 to S24 | 148873-91-0 | [148873-91-0]-S1 to S24 |
| S1 to S24 | 1304130-05-9 | [1304130-05-9]-S1 to S24 |
| S1 to S24 | 1314564-37-8 | [1314564-37-8]-S1 to S24 |
| S1 to S24 | 1449401-77-7 | [1449401-77-7]-S1 to S24 |
| S1 to S24 | 2095370-49-1 | [2095370-49-1]-S1 to S24 |
| S1 to S24 | 122447-72-7 | [122447-72-7]-S1 to S24 |
| S1 to S24 | 845457-53-6 | [845457-53-6]-S1 to S24 |
| S1 to S24 | 1182175-15-0 | [1182175-15-0]-S1 to S24 |
| S1 to S24 | 1263295-29-9 | [1263295-29-9]-S1 to S24 |
| S1 to S24 | 1314565-30-4 | [1314565-30-4]-S1 to S24 |
| S1 to S24 | 2271144-10-4 | [2271144-10-4]-S1 to S24 |
| S1 to S24 | 2411311-91-4 | [2411311-91-4]-S1 to S24 |
| S1 to S24 | 2036123-44-9 | [2036123-44-9]-S1 to S24 |
| S1 to S24 | 1314653-28-5 | [1314653-28-5]-S1 to S24 |
| S1 to S24 | 1314564-45-8 | [1314564-45-8]-S1 to S24 |
| S1 to S24 | 1314653-29-6 | [1314653-29-6]-S1 to S24 |
| S1 to S24 | 888950-02-5 | [888950-02-5]-S1 to S24 |
| S1 to S24 | 1314563-60-4 | [1314563-60-4]-S1 to S24 |
| S1 to S24 | 944801-21-2 | [944801-21-2]-S1 to S24 |
| S1 to S24 | 944801-28-9 | [944801-28-9]-S1 to S24 |
| S1 to S24 | 1092391-01-6 | [1092391-01-6]-S1 to S24 |
| S1 to S24 | 1277187-32-2 | [1277187-32-2]-S1 to S24 |
| S1 to S24 | 1314653-16-1 | [1314653-16-1]-S1 to S24 |
| S1 to S24 | 1314653-64-9 | [1314653-64-9]-S1 to S24 |
| S1 to S24 | 1359876-41-7 | [1359876-41-7]-S1 to S24 |
| S1 to S24 | 1492925-89-9 | [1492925-89-9]-S1 to S24 |
| S1 to S24 | 1623149-25-6 | [1623149-25-6]-S1 to S24 |
| S1 to S24 | 2420556-10-9 | [2420556-10-9]-S1 to S24 |
| S1 to S24 | 2097255-54-2 | [2097255-54-2]-S1 to S24 |
| S1 to S24 | 1673542-07-8 | [1673542-07-8]-S1 to S24 |
| S1 to S24 | 2056912-06-0 | [2056912-06-0]-S1 to S24 |
| S1 to S24 | 1273319-86-0 | [1273319-86-0]-S1 to S24 |
| S1 to S24 | 1314562-66-7 | [1314562-66-7]-S1 to S24 |
| S1 to S24 | 1314562-79-2 | [1314562-79-2]-S1 to S24 |
| S1 to S24 | 474688-74-9 | [474688-74-9]-S1 to S24 |
| S1 to S24 | 1492925-90-2 | [1492925-90-2]-S1 to S24 |
| S1 to S24 | 1492925-96-8 | [1492925-96-8]-S1 to S24 |
| S1 to S24 | 1381927-19-0 | [1381927-19-0]-S1 to S24 |
| S1 to S24 | 1898202-64-6 | [1898202-64-6]-S1 to S24 |
| S1 to S24 | 1314562-72-5 | [1314562-72-5]-S1 to S24 |
| S1 to S24 | 1314564-59-4 | [1314564-59-4]-S1 to S24 |
| S1 to S24 | 1572298-39-5 | [1572298-39-5]-S1 to S24 |
| S1 to S24 | 1314563-82-0 | [1314563-82-0]-S1 to S24 |
| S1 to S24 | 522616-11-1 | [522616-11-1]-S1 to S24 |
| S1 to S24 | 1012311-18-0 | [1012311-18-0]-S1 to S24 |
| S1 to S24 | 1062555-52-5 | [1062555-52-5]-S1 to S24 |
| S1 to S24 | 1062556-01-7 | [1062556-01-7]-S1 to S24 |
| S1 to S24 | 1188095-54-6 | [1188095-54-6]-S1 to S24 |
| S1 to S24 | 1197361-53-0 | [1197361-53-0]-S1 to S24 |
| S1 to S24 | 1204238-03-8 | [1204238-03-8]-S1 to S24 |
| S1 to S24 | 1314653-19-4 | [1314653-19-4]-S1 to S24 |
| S1 to S24 | 2089692-32-8 | [2089692-32-8]-S1 to S24 |
| S1 to S24 | 2219351-13-8 | [2219351-13-8]-S1 to S24 |
| S1 to S24 | 2413696-91-8 | [2413696-91-8]-S1 to S24 |
| S1 to S24 | 2423091-14-7 | [2423091-14-7]-S1 to S24 |
| S1 to S24 | 2423091-19-2 | [2423091-19-2]-S1 to S24 |
| S1 to S24 | 1403665-09-7 | [1403665-09-7]-S1 to S24 |
| S1 to S24 | 910864-19-6 | [910864-19-6]-S1 to S24 |
| S1 to S24 | 1314653-11-6 | [1314653-11-6]-S1 to S24 |
| S1 to S24 | 2097263-62-0 | [2097263-62-0]-S1 to S24 |
| S1 to S24 | 1314557-45-3 | [1314557-45-3]-S1 to S24 |
| S1 to S24 | 1445948-69-5 | [1445948-69-5]-S1 to S24 |
| S1 to S24 | 2446780-52-3 | [2446780-52-3]-S1 to S24 |
| S1 to S24 | 2095111-26-3 | [2095111-26-3]-S1 to S24 |
| S1 to S24 | 400607-65-0 | [400607-65-0]-S1 to S24 |
| S1 to S24 | 1062556-05-1 | [1062556-05-1]-S1 to S24 |
| S1 to S24 | 1172087-81-8 | [1172087-81-8]-S1 to S24 |
| S1 to S24 | 2446780-51-2 | [2446780-51-2]-S1 to S24 |
| S1 to S24 | 2309274-31-3 | [2309274-31-3]-S1 to S24 |
| S1 to S24 | 2319594-91-5 | [2319594-91-5]-S1 to S24 |
| S1 to S24 | 2194590-65-1 | [2194590-65-1]-S1 to S24 |
| S1 to S24 | 2271144-11-5 | [2271144-11-5]-S1 to S24 |
| S1 to S24 | 1384281-78-0 | [1384281-78-0]-S1 to S24 |
| S1 to S24 | 866609-90-7 | [866609-90-7]-S1 to S24 |
| S1 to S24 | 949925-54-6 | [949925-54-6]-S1 to S24 |
| S1 to S24 | 949925-67-1 | [949925-67-1]-S1 to S24 |
| S1 to S24 | 910244-27-8 | [910244-27-8]-S1 to S24 |
| S1 to S24 | 522616-09-7 | [522616-09-7]-S1 to S24 |
| S1 to S24 | 2093406-34-7 | [2093406-34-7]-S1 to S24 |
| S1 to S24 | 2096373-43-0 | [2096373-43-0]-S1 to S24 |
| S1 to S24 | 2096373-45-2 | [2096373-45-2]-S1 to S24 |
| S1 to S24 | 2096373-46-3 | [2096373-46-3]-S1 to S24 |
| S1 to S24 | 2411319-74-7 | [2411319-74-7]-S1 to S24 |
| S1 to S24 | 2414932-91-3 | [2414932-91-3]-S1 to S24 |
| S1 to S24 | 1062556-10-8 | [1062556-10-8]-S1 to S24 |
| S1 to S24 | 1062556-22-2 | [1062556-22-2]-S1 to S24 |
| S1 to S24 | 1172093-53-6 | [1172093-53-6]-S1 to S24 |
| S1 to S24 | 2121653-62-9 | [2121653-62-9]-S1 to S24 |
| S1 to S24 | 1616658-45-7 | [1616658-45-7]-S1 to S24 |
| S1 to S24 | 121848-75-7 | [121848-75-7]-S1 to S24 |
| S1 to S24 | 523-27-3 | [523-27-3]-S1 to S24 |

Bromophenanthrenes

| | | |
|---|---|---|
| S1 to S24 | 1132778-00-7 | [1132778-00-7]-S1 to S24 |
| S1 to S24 | 205581-26-2 | [205581-26-2]-S1 to S24 |
| S1 to S24 | 2294929-90-9 | [2294929-90-9]-S1 to S24 |
| S1 to S24 | 15810-15-8 | [15810-15-8]-S1 to S24 |
| S1 to S24 | 1266612-80-9 | [1266612-80-9]-S1 to S24 |
| S1 to S24 | 2448364-61-0 | [2448364-61-0]-S1 to S24 |
| S1 to S24 | 1448787-83-4 | [1448787-83-4]-S1 to S24 |
| S1 to S24 | 1345860-66-3 | [1345860-66-3]-S1 to S24 |
| S1 to S24 | 1306616-35-2 | [1306616-35-2]-S1 to S24 |
| S1 to S24 | 2414209-05-3 | [2414209-05-3]-S1 to S24 |
| S1 to S24 | 877128-08-0 | [877128-08-0]-S1 to S24 |
| S1 to S24 | 2085735-37-9 | [2085735-37-9]-S1 to S24 |
| S1 to S24 | 2414209-06-4 | [2414209-06-4]-S1 to S24 |
| S1 to S24 | 19462-79-4 | [19462-79-4]-S1 to S24 |

Bromotriphenylenes

| | | |
|---|---|---|
| S1 to S24 | 1616514-40-9 | [1616514-40-9]-S1 to S24 |
| S1 to S24 | 1616514-39-6 | [1616514-39-6]-S1 to S24 |
| S1 to S24 | 1616514-38-5 | [1616514-38-5]-S1 to S24 |
| S1 to S24 | 1616514-37-4 | [1616514-37-4]-S1 to S24 |
| S1 to S24 | 1616514-36-3 | [1616514-36-3]-S1 to S24 |
| S1 to S24 | 1616514-35-2 | [1616514-35-2]-S1 to S24 |
| S1 to S24 | 1616514-34-1 | [1616514-34-1]-S1 to S24 |
| S1 to S24 | 1616514-33-0 | [1616514-33-0]-S1 to S24 |
| S1 to S24 | 74897-21-5 | [74897-21-5]-S1 to S24 |
| S1 to S24 | 19111-87-6 | [19111-87-6]-S1 to S24 |
| S1 to S24 | 24253-52-9 | [24253-52-9]-S1 to S24 |
| S1 to S24 | 1616514-01-2 | [1616514-01-2]-S1 to S24 |
| S1 to S24 | 1616514-02-3 | [1616514-02-3]-S1 to S24 |
| S1 to S24 | 1616514-03-4 | [1616514-03-4]-S1 to S24 |
| S1 to S24 | 1616514-14-7 | [1616514-14-7]-S1 to S24 |
| S1 to S24 | 1616514-08-9 | [1616514-08-9]-S1 to S24 |
| S1 to S24 | 1616514-17-0 | [1616514-17-0]-S1 to S24 |
| S1 to S24 | 1616514-20-5 | [1616514-20-5]-S1 to S24 |

Bromopyrenes

| | | |
|---|---|---|
| S1 to S24 | 1732-26-9 | [1732-26-9]-S1 to S24 |
| S1 to S24 | 1640101-48-9 | [1640101-48-9]-S1 to S24 |
| S1 to S24 | 1640101-46-7 | [1640101-46-7]-S1 to S24 |
| S1 to S24 | 1640101-40-1 | [1640101-40-1]-S1 to S24 |
| S1 to S24 | 1640101-39-8 | [1640101-39-8]-S1 to S24 |
| S1 to S24 | 1640101-38-7 | [1640101-38-7]-S1 to S24 |
| S1 to S24 | 1640101-37-6 | [1640101-37-6]-S1 to S24 |
| S1 to S24 | 1640101-34-3 | [1640101-34-3]-S1 to S24 |
| S1 to S24 | 1640101-31-0 | [1640101-31-0]-S1 to S24 |
| S1 to S24 | 1640101-30-9 | [1640101-30-9]-S1 to S24 |
| S1 to S24 | 1448535-13-4 | [1448535-13-4]-S1 to S24 |
| S1 to S24 | 1448535-12-3 | [1448535-12-3]-S1 to S24 |
| S1 to S24 | 1448535-11-2 | [1448535-11-2]-S1 to S24 |
| S1 to S24 | 1448535-09-8 | [1448535-09-8]-S1 to S24 |
| S1 to S24 | 2454312-40-2 | [2454312-40-2]-S1 to S24 |
| S1 to S24 | 2454312-39-9 | [2454312-39-9]-S1 to S24 |
| S1 to S24 | 2454312-38-8 | [2454312-38-8]-S1 to S24 |
| S1 to S24 | 2162125-87-1 | [2162125-87-1]-S1 to S24 |
| S1 to S24 | 2162125-86-0 | [2162125-86-0]-S1 to S24 |
| S1 to S24 | 2096506-47-5 | [2096506-47-5]-S1 to S24 |
| S1 to S24 | 2095803-36-2 | [2095803-36-2]-S1 to S24 |
| S1 to S24 | 2095184-23-7 | [2095184-23-7]-S1 to S24 |
| S1 to S24 | 1714-29-0 | [1714-29-0]-S1 to S24 |
| S1 to S24 | 294881-47-3 | [294881-47-3]-S1 to S24 |
| S1 to S24 | 2351281-21-3 | [2351281-21-3]-S1 to S24 |
| S1 to S24 | 929099-50-3 | [929099-50-3]-S1 to S24 |
| S1 to S24 | 870774-30-4 | [870774-30-4]-S1 to S24 |
| S1 to S24 | 1622011-24-8 | [1622011-24-8]-S1 to S24 |
| S1 to S24 | 1198003-30-3 | [1198003-30-3]-S1 to S24 |
| S1 to S24 | 1198003-40-5 | [1198003-40-5]-S1 to S24 |
| S1 to S24 | 1672689-09-6 | [1672689-09-6]-S1 to S24 |
| S1 to S24 | 1808074-54-5 | [1808074-54-5]-S1 to S24 |
| S1 to S24 | 1416893-92-9 | [1416893-92-9]-S1 to S24 |
| S1 to S24 | 1622010-97-2 | [1622010-97-2]-S1 to S24 |
| S1 to S24 | 2162125-86-0 | [2162125-86-0]-S1 to S24 |
| S1 to S24 | 870774-28-0 | [870774-28-0]-S1 to S24 |
| S1 to S24 | 1672689-04-1 | [1672689-04-1]-S1 to S24 |
| S1 to S24 | 38037-54-6 | [38037-54-6]-S1 to S24 |
| S1 to S24 | 1808074-55-6 | [1808074-55-6]-S1 to S24 |
| S1 to S24 | 1808074-52-3 | [1808074-52-3]-S1 to S24 |
| S1 to S24 | 1942086-15-4 | [1942086-15-4]-S1 to S24 |
| S1 to S24 | 1942086-04-5 | [1942086-04-5]-S1 to S24 |
| S1 to S24 | 1942085-93-9 | [1942085-93-9]-S1 to S24 |
| S1 to S24 | 1714-27-8 | [1714-27-8]-S1 to S24 |
| S1 to S24 | 1610364-51-6 | [1610364-51-6]-S1 to S24 |
| S1 to S24 | 2307445-47-0 | [2307445-47-0]-S1 to S24 |
| S1 to S24 | 1672689-11-0 | [1672689-11-0]-S1 to S24 |
| S1 to S24 | 1607017-04-8 | [1607017-04-8]-S1 to S24 |
| S1 to S24 | 2413555-98-1 | [2413555-98-1]-S1 to S24 |

Bromochrysenes

| | | |
|---|---|---|
| S1 to S24 | 2446780-44-3 | [2446780-44-3]-S1 to S24 |
| S1 to S24 | 1763545-11-3 | [1763545-11-3]-S1 to S24 |
| S1 to S24 | 345219-30-9 | [345219-30-9]-S1 to S24 |
| S1 to S24 | 2417805-34-4 | [2417805-34-4]-S1 to S24 |
| S1 to S24 | 1172095-96-3 | [1172095-96-3]-S1 to S24 |
| S1 to S24 | 1579998-22-3 | [1579998-22-3]-S1 to S24 |
| S1 to S24 | 7397-93-5 | [7397-93-5]-S1 to S24 |
| S1 to S24 | 21248-00-0 | [21248-00-0]-S1 to S24 |
| S1 to S24 | 122048-53-7 | [122048-53-7]-S1 to S24 |
| S1 to S24 | 1096686-64-4 | [1096686-64-4]-S1 to S24 |
| S1 to S24 | 1231774-99-4 | [1231774-99-4]-S1 to S24 |
| S1 to S24 | 1100078-86-1 | [1100078-86-1]-S1 to S24 |
| S1 to S24 | 1673534-11-3 | [1673534-11-3]-S1 to S24 |
| S1 to S24 | 1579998-22-3 | [1579998-22-3]-S1 to S24 |
| S1 to S24 | 1875101-96-4 | [1875101-96-4]-S1 to S24 |
| S1 to S24 | 76670-38-7 | [76670-38-7]-S1 to S24 |
| S1 to S24 | 55120-48-4 | [55120-48-4]-S1 to S24 |
| S1 to S24 | 1261081-96-2 | [1261081-96-2]-S1 to S24 |
| S1 to S24 | 56158-60-2 | [56158-60-2]-S1 to S24 |
| S1 to S24 | 1265909-78-1 | [1265909-78-1]-S1 to S24 |
| S1 to S24 | 1231774-99-4 | [1231774-99-4]-S1 to S24 |
| S1 to S24 | 76670-41-2 | [76670-41-2]-S1 to S24 |

Bromophenalenes

| | | |
|---|---|---|
| S1 to S24 | 13438-50-1 | [13438-50-1]-S1 to S24 |
| S1 to S24 | 138816-09-8 | [138816-09-8]-S1 to S24 |
| S1 to S24 | 120362-70-1 | [120362-70-1]-S1 to S24 |
| S1 to S24 | 862508-20-1 | [862508-20-1]-S1 to S24 |
| S1 to S24 | 370098-12-7 | [370098-12-7]-S1 to S24 |

Bromoperylenes

| | | |
|---|---|---|
| S1 to S24 | 2246953-20-6 | [2246953-20-6]-S1 to S24 |
| S1 to S24 | 23683-68-3 | [23683-68-3]-S1 to S24 |
| S1 to S24 | 109465-97-6 | [109465-97-6]-S1 to S24 |
| S1 to S24 | 138206-23-2 | [138206-23-2]-S1 to S24 |

The expression 11314563-82-01-S1 to S24″ represents 24 different products that are each obtained by reacting compound CAS No. 1314563-82-0 with the boronic esters S1 to S24 specified in the Examples. The same applies to the other products specified in the above table.

It is possible by these methods, if necessary followed by purification, for example recrystallization or sublimation, to obtain the compounds of the invention in high purity, preferably more than 99% (determined by means of ¹H NMR and/or HPLC).

The compounds of the invention or the compounds usable in accordance with the invention may also be mixed with a polymer. It is likewise possible to incorporate these compounds covalently into a polymer. This is especially possible with compounds substituted by reactive leaving groups such as bromine, iodine, chlorine, boronic acid or boronic ester, or by reactive polymerizable groups such as olefins or oxetanes. These may find use as monomers for production of corresponding oligomers, dendrimers or polymers. The oligomerization or polymerization is preferably effected via the halogen functionality or the boronic acid functionality or via the polymerizable group. It is additionally possible to crosslink the polymers via groups of this kind. The compounds and polymers of the invention may be used in the form of a crosslinked or uncrosslinked layer.

The invention therefore further provides oligomers, polymers or dendrimers containing one or more of the above-detailed structures of the formulae (I), (II), (III), (IV), (V), (VI) and preferred embodiments of these formulae or compounds of the invention, wherein one or more bonds of the compounds of the invention or of the structures of the formulae (I), (II), (III), (IV), (V), (VI) and preferred embodiments of that formula to the polymer, oligomer or dendrimer are present. According to the linkage of the structures of the formulae (I), (II), (III), (IV), (V), (VI) and preferred embodiments of this formula or of the compounds, these therefore form a side chain of the oligomer or polymer or are bonded within the main chain. The polymers, oligomers or dendrimers may be conjugated, partly conjugated or nonconjugated. The oligomers or polymers may be linear, branched or dendritic. For the repeat units of the compounds of the invention in oligomers, dendrimers and polymers, the same preferences apply as described above.

For preparation of the oligomers or polymers, the monomers of the invention are homopolymerized or copolymerized with further monomers. Preference is given to copolymers wherein the units of formulae (I), (II), (III), (IV), (V), (VI) or the preferred embodiments recited above and hereinafter are present to an extent of 0.01 to 99.9 mol %, preferably 5 to 90 mol %, more preferably 20 to 80 mol %. Suitable and preferred comonomers which form the polymer base skeleton are chosen from fluorenes (for example according to EP 842208 or WO 2000/022026), spirobifluorenes (for example according to EP 707020, EP 894107 or WO 2006/061181), paraphenylenes (for example according to WO 92/18552), carbazoles (for example according to WO 2004/070772 or WO 2004/113468), thiophenes (for example according to EP 1028136), dihydrophenanthrenes (for example according to WO 2005/014689), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers may contain still further units, for example hole transport units, especially those based on triarylamines, and/or electron transport units.

Additionally of particular interest are compounds of the invention which feature a high glass transition temperature. In this connection, preference is given especially to compounds of the invention comprising structures of the formulae (I), (II), (III), (IV), (V), (VI) or the preferred embodiments recited above and hereinafter which have a glass transition temperature of at least 70° C., more preferably of at least 110° C., even more preferably of at least 125° C. and especially preferably of at least 150° C., determined in accordance with DIN 51005 (2005-08 version).

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. If the further compound comprises a solvent, this mixture is referred to herein as formulation. The further compound may alternatively be at least one further organic or inorganic compound which is likewise used in the electronic device, for example an emitter and/or a matrix material, where these compounds differ from the compounds of the invention. Suitable emitters and matrix materials are listed at the back in connection with the organic electroluminescent device. The further compound may also be polymeric.

The present invention therefore still further provides a composition comprising a compound of the invention and at least one further organofunctional material. Functional materials are generally the organic or inorganic materials introduced between the anode and cathode. Preferably, the organofunctional material is selected from the group consisting of fluorescent emitters, phosphorescent emitters, emitters that exhibit TADF (thermally activated delayed fluorescence), host materials, electron transport materials, electron injection materials, hole conductor materials, hole injection materials, electron blocker materials, hole blocker materials, wide bandgap materials and n-dopants.

The present invention still further provides an electronic device comprising at least one compound of the invention. An electronic device in the context of the present invention is a device comprising at least one layer comprising at least one organic compound. The component may also comprise inorganic materials or else layers formed entirely from inorganic materials.

The electronic device is preferably selected from the group consisting of organic electroluminescent devices (OLEDs, sOLED, PLEDs, LECs, etc.), preferably organic light-emitting diodes (OLEDs), organic light-emitting diodes based on small molecules (sOLEDs), organic light-emitting diodes based on polymers (PLEDs), light-emitting electrochemical cells (LECs), organic laser diodes (O-laser), organic plasmon-emitting devices (D. M. Koller et al., Nature Photonics 2008, 1-4), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs) and organic electrical sensors, preferably organic electroluminescent devices (OLEDs, sOLED, PLEDs, LECs, etc.), more preferably organic light-emitting diodes (OLEDs), organic light-emitting diodes based on small molecules (sOLEDs), organic light-emitting diodes based on polymers (PLEDs), especially phosphorescent OLEDs.

A preferred embodiment of an electronic device comprises at least one, preferably exactly one, antideposition layer. The effect of an antideposition layer is that layers applied subsequently to said layer are formed or deposited to a limited degree, preferably not at all. Accordingly, an antideposition layer is preferably not complete or continuous, but rather preferably has a structure. By virtue of this structure, substances applied subsequently, for example metals, can come into contact with layers applied beforehand. An antideposition layer serves, for example, for production of auxiliary electrodes detailed above and hereinafter that bring about a reduction in the resistance of an electronic device.

The antideposition layer may be produced, for example, by means of a shadowmask with mask openings. What is especially advantageous here is that the mask thus used can easily be cleaned and reused. Solvents suitable for this purpose are set out above, and so reference is made thereto, and NMP may be used with preference. The solvent may optionally be used at elevated temperature.

A preferred electronic device comprises at least one compound, the use of which is defined above, a compound comprising at least one structure of the formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7), or an oligomer, polymer or dendrimer based on these compounds or a composition comprising at least one of these compounds, where the electronic device comprises an antideposition layer, where the compound, the use of which is defined above, the compound comprising at least one structure of the formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7) or an oligomer, polymer or dendrimer based on these compounds or a composition comprising at least one of these compounds is present in said antideposition layer.

The antideposition layer preferably consists of one or more compounds, the use of which is defined above, of one or more of the compounds comprising at least one structure of the formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7), or one or more of the oligomers, polymers or dendrimers based on these compounds or a composition comprising at least one of these compounds.

The antideposition layer more preferably consists of one or more compounds, the use of which is defined above, or of one or more of the compounds comprising at least one structure of the formulae (I), (I-1) to (I-7), (Ia-1) to (Ia-7), (Ib-1) to (Ib-7), (Ic-1) to (Ic-7), (II), (II-1) to (II-7), (IIa-1) to (IIa-7), (IIb-1) to (IIb-7), (IIc-1) to (IIc-7), (III), (III-1) to (III-7), (IIIa-1) to (IIIa-7), (IIIb-1) to (IIIb-7), (IIIc-1) to (IIIc-7), (IV), (IV-1) to (IV-7), (IVa-1) to (IVa-7), (IVb-1) to (IVb-7), (IVc-1) to (IVc-7), (V), (V-1) to (V-7), (Va-1) to (Va-7), (Vb-1) to (Vb-7), (Vc-1) to (Vc-7), (VI), (VI-1) to (VI-7), (VIa-1) to (VIa-7), (VIb-1) to (VIb-7) and (VIc-1) to (VIc-7).

In a preferred embodiment, the antideposition layer may be obtained at a deposition rate preferably within a range from 0.1 to 100 angstrOms/second (A/s), more preferably in the range from 1 to 50 A/s and especially preferably in the range from 2 to 20 A/s. The measurement is typically effected with a tooled (calibrated) crystal oscillator.

An electronic device comprises cathode, anode and at least one functional layer. An electronic device of the invention, as well as these layers, preferably comprises an antideposition layer as described in detail above and hereinafter. The antideposition layer especially serves to produce an electrically conductive structure, preferably auxiliary electrode. It may preferably be the case that the antideposition layer serves to produce an auxiliary cathode. In a preferred embodiment, the antideposition layer may be provided between emission layer and cathode.

Electrically conductive substances that serve to produce electrically conductive units, for example, especially auxiliary electrodes, may in principle include all materials that are used for production of an anode or cathode. These materials are preferably applied by evaporation methods, and so preference is given to using metals, metal alloys or semimetals.

Preferred metals, metal alloys or semimetals are notable for good evaporability and high conductivity. The following are among those suitable here: alkali metals, especially Li, Na, K; alkaline earth metals, especially Be, Mg, Ca, Sr, Ba; metals of main group 3, especially Al, Ga, In; metals or metalloids of main group 4, especially Si, Ge, Sn, Bi; transition metals, preferably Cu, Ag, Au, Zn; lanthanides, preferably Yb. These metals may be used individually or as an alloy of 2, 3, 4 or more components. These alloys may be obtained by methods including evaporation or coevaporation of the mixture at the eutectic point, such that these alloys are obtained directly as a structured layer in the production of the electronic device.

Preferred materials, especially metals or metal alloys, that may be used for production of preferred cathodes are notable for a work function which is preferably in the range from 1.7 to 5.5 eV, more preferably in the range from 2.0 to 5.0 eV, especially preferably in the range from 2.5 to 4.5 eV.

In a preferred embodiment, the electrically conductive structure, preferably the auxiliary electrode, may be obtained at a deposition rate preferably within a range from 0.1 to 100 angstrOms/second (A/s), more preferably in the range from 1 to 50 A/s and especially preferably in the range from 2 to 20 A/s. The measurement is typically effected with a tooled (calibrated) crystal oscillator.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

A preferred mixture of an emitter and a matrix material contains between 99% and 1% by volume, preferably between 98% and 10% by volume, more preferably between 97% and 60% by volume and especially between 95% and 80% by volume of matrix material, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

Suitable matrix materials are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

In addition, the co-host used may be a compound that does not take part in charge transport to a significant degree, if at all, as described, for example, in WO 2010/108579. Especially suitable as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

In a preferred configuration, an emitter is preferably used in combination with one or more phosphorescent materials (triplet emitters) and/or a compound which is a TADF (thermally activated delayed fluorescence) host material. Preference is given here to forming a hyperfluorescence and/or hyperphosphorescence system.

WO 2015/091716 A1 and WO 2016/193243 A1 disclose OLEDs containing both a phosphorescent compound and a fluorescent emitter in the emission layer, where the energy is transferred from the phosphorescent compound to the fluorescent emitter (hyperphosphorescence). In this context, the phosphorescent compound accordingly behaves as a host material. As the person skilled in the art knows, host materials have higher singlet and triplet energies as compared to the emitters in order that the energy from the host material can also be transferred to the emitter with maximum efficiency. The systems disclosed in the prior art have exactly such an energy relation.

Phosphorescence in the context of this invention is understood to mean luminescence from an excited state having higher spin multiplicity, i.e. a spin state>1, especially from an excited triplet state. In the context of this application, all luminescent complexes with transition metals or lanthanides, especially all iridium, platinum and copper complexes, shall be regarded as phosphorescent compounds.

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186, WO 2018/001990, WO 2018/019687, WO 2018/019688, WO 2018/041769, WO 2018/054798, WO 2018/069196, WO 2018/069197, WO 2018/069273, WO 2018/178001, WO 2018/177981, WO 2019/020538, WO 2019/115423, WO 2019/158453 and WO 2019/179909. In general, all phosphorescent complexes as used for phosphorescent electroluminescent devices according to the prior art and as known to those skilled in the art in the field of organic electroluminescence are suitable, and the person skilled in the art will be able to use further phosphorescent complexes without exercising inventive skill.

The process referred to as thermally activated delayed fluorescence (TADF) is described, for example, by B. H. Uoyama et al., Nature 2012, Vol. 492, 234. In order to enable this process, a comparatively small singlet-triplet separation ΔE(S₁−T₁) of less than about 2000 cm⁻¹, for example, is needed in the emitter. In order to open up the T₁→S₁ transition which is spin-forbidden in principle, as well as the emitter, it is possible to provide a further compound in the matrix that has strong spin-orbit coupling, such that intersystem crossing is enabled via the spatial proximity and the interaction which is thus possible between the molecules, or the spin-orbit coupling is generated by means of a metal atom present in the emitter.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981. What should be noted here is that the antideposition layer is preferably not complete, such that the electrodes are in direct contact with the further layers via applied metal.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the compounds usable in accordance with the invention or the compounds of the invention or the preferred embodiments recited above.

Additionally preferred is an organic electroluminescent device, characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

Formulations for application of a compound of formula (I), (II), (III), (IV), (V), (VI) or the preferred embodiments thereof that are set out above are novel. The present invention therefore further provides a formulation comprising at least one solvent and a compound of formula (I) or the preferred embodiments thereof that are set out above.

In addition, hybrid methods are possible, in which, for example, one or more layers are applied from solution and one or more further layers are applied by vapour deposition.

These methods are known in general terms to those skilled in the art and can be applied by those skilled in the art without exercising inventive skill to organic electroluminescent devices comprising the compounds of the invention.

The compounds of the invention and the organic electroluminescent devices of the invention have the particular feature of an improved lifetime over the prior art. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

The electronic devices of the invention, especially organic electroluminescent devices, are notable for one or more of the following surprising advantages over the prior art:
1. The compounds usable in accordance with the invention or compounds of formula (I), (II), (III), (IV), (V), (VI), or the preferred embodiments that are set out above and hereinafter, may be applied by gas deposition methods at very different evaporation rates. It is possible thereby to produce preferred electronic devices in a very simple, reliable and inexpensive manner.
2. The compounds usable in accordance with the invention or compounds of formula (I), (II), (III), (IV), (V), (VI), or the preferred embodiments that are set out above and hereinafter, may be applied to very different layers via gas deposition methods, and in this case show excellent structuring capacity for different metals/metal alloys. It is possible thereby to produce preferred electronic devices with very different structure in a very simple, reliable and inexpensive manner.
3. The compounds usable in accordance with the invention or compounds of formula (I), (II), (III), (IV), (V), (VI), or the preferred embodiments that are set out above and hereinafter, are notable for excellent solubility in many solvents. As a result, it is possible to clean the shadowmasks to be used with preference for structuring easily and inexpensively. In this connection, it should be stated that the shadowmasks used to date for structuring have to be produced individually for every electronic device and are correspondingly costly. If these masks are used to structure evaporated metal, these masks will become unusable after a short time since deposited metal leads to a reduction in size or to closure of the openings provided in the mask. This metal deposited on the mask cannot be removed from the mask. By contrast, proportions of compounds usable in accordance with the invention or compounds of formula (I), (II), (III), (IV), (V), (VI), or the preferred embodiments that have been set out above and hereinafter, that have been deposited on a mask, on account of high solubility in solvents, can be dissolved quickly, reliably and inexpensively in small amounts of solvents and hence removed from the mask. This enables cleaning and reuse of these masks in a particularly simple manner.
4. With compounds usable in accordance with the invention or compounds of formula (I), (II), (III), (IV), (V), (VI), or the preferred embodiments that have been set out above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. Compounds usable in accordance with the invention or compounds of formula (I), (II), (III), (IV), (V), (VI), or the preferred embodiments that have been set out above and hereinafter, exhibit an excellent glass film formation.
6. Electronic devices, especially organic electroluminescent devices comprising compounds usable in accordance with the invention or compounds of formula (I), (II), (III), (IV), (V), (VI), or the preferred embodiments that have been set out above and hereinafter, may have very narrow emission bands having very low FWHM (Full Width Half Maximum) values, and enable particularly colour-pure emission, recognizable by the low CIE y values. It should be stated here that the compounds usable in accordance with the invention or compounds of formula (I), (II), (III), (IV), (V), (VI), or the preferred embodiments that have been set out above and hereinafter, do not have any adverse effects on the emission bands.
7. Electronic devices, especially organic electroluminescent devices, comprising compounds usable in accordance with the invention or compounds of formula (I), (II), (III), (IV), (V), (VI), or the preferred embodiments that have been set out above and hereinafter, especially for structuring of at least one functional layer, have very good lifetime. It should be stated here that the compounds usable in accordance with the invention or compounds of formula (I), (II), (III), (IV), (V), (VI), or the preferred embodiments that have been set out above and hereinafter, do not have any adverse effects on lifetime. Accordingly, devices of the invention may have low roll-off, i.e. a small drop in power efficiency of the device at high luminances.
8. Electronic devices, especially organic electroluminescent devices, comprising compounds usable in accordance with the invention or compounds of formula (I), (II), (III), (IV), (V), (VI), or the preferred embodiments that have been set out above and hereinafter, for structuring of at least one functional layer may have excellent efficiency. It should be stated here that the compounds usable in accordance with the invention or compounds of formula (I), (II), (III), (IV), (V), (VI), or the preferred embodiments that have been set out above and hereinafter, do not have any adverse effects on efficiency. Moreover, the compounds usable in accordance with the invention or compounds of formula (I), or the preferred embodiments set out above and hereinafter, contribute indirectly to a low operating voltage in electronic devices via the creation of an electrically conductive structure.
9. The compounds usable in accordance with the invention or compounds of formula (I), (II), (III), (IV), (V), (VI), or the preferred embodiments that are set out above and hereinafter, show high stability and lifetime.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### WORKING EXAMPLES

The syntheses which follow, unless stated otherwise, are conducted under a protective gas atmosphere in dried solvents. The solvents and reagents can be purchased, for example, from Sigma-ALDRICH or ABCR. The respective figures in square brackets or the numbers quoted for individual compounds relate to the CAS numbers of the compounds known from the literature. In the case of compounds that can have multiple enantiomeric, diastereomeric or tautomeric forms, one form is shown in a representative manner.

### Synthesis of the Compounds

### Example 1

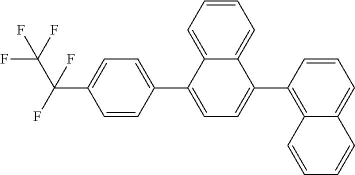 [SMILES: FC(F)(F)C(F)(F)c1ccc(-c2ccc(-c3cccc4ccccc34)c3ccccc23)cc1]

A mixture of 32.2 g (100 mmol) of 4,4,5,5-tetramethyl-2-[4-(1,1,2,2,2-pentafluoroethyl)phenyl]-1,3,2-dioxaborolane [2088974-50-7] (boronic ester 51), 33.3 g (100 mmol) of 4-bromo-1,1′-binaphthalene [49610-33-5], 31.8 g (300 mmol) of sodium carbonate [497-19-8], 1.48 g (2 mmol) of bis(tricyclohexylphosphine)palladium(II) chloride [29934-17-6], 5 drops of hydrazine hydrate [7803-57-8], 300 ml of toluene, 100 ml of isopropanol and 300 ml of water is stirred at 80° C. for 16 h. After cooling, the solids are filtered off with suction, and the filtrate is washed twice with 200 ml each time of water and twice with 100 ml each time of methanol, and dried under reduced pressure. The solids are taken up in 300 ml of dichloromethane and filtered through a silica gel bed in the form of a DCM slurry, 200 ml of methanol is added to the filtrate, and the mixture is concentrated to a volume of about 100 ml under reduced pressure. The crystallized product is filtered off with suction and dried under reduced pressure. Purification is effected via hot extraction crystallization three times from acetonitrile, or by chromatography on silica gel and subsequent fractional sublimation. Yield: 30.6 g (68 mmol), 68%; purity: >99.5% by HPLC.

The following compounds can be prepared analogously:

| Ex.. | Reactants | Product | Yield |
|---|---|---|---|
| 2 | 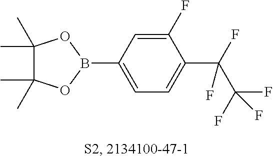 [SMILES: CC1(C)OB(c2ccc(C(F)(F)C(F)(F)F)c(F)c2)OC1(C)C] | 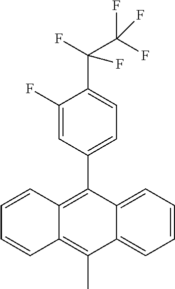 [SMILES: Cc1c2ccccc2c(-c2ccc(C(F)(F)C(F)(F)F)c(F)c2)c2ccccc12] | 60% |
| | 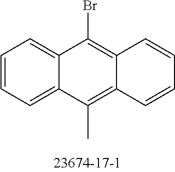 [SMILES: Cc1c2ccccc2c(Br)c2ccccc12] | | |
| 3 | 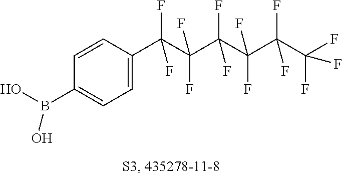 [SMILES: OB(O)c1ccc(C(F)(F)C(F)(F)C(F)(F)C(F)(F)C(F)(F)C(F)(F)F)cc1] | 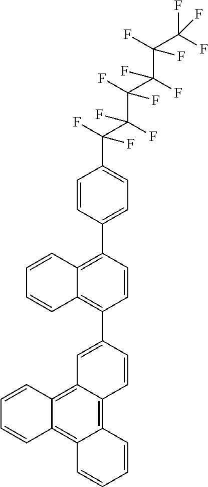 [SMILES: FC(F)(F)C(F)(F)C(F)(F)C(F)(F)C(F)(F)C(F)(F)c1ccc(-c2ccc(-c3ccc4c5ccccc5c5ccccc5c4c3)c3ccccc23)cc1] | 63% |
| | 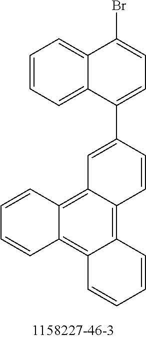 [SMILES: Brc1ccc(-c2ccc3c4ccccc4c4ccccc4c3c2)c2ccccc12] | | |
| 4 | 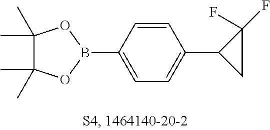 [SMILES: CC1(C)OB(c2ccc(C3CC3(F)F)cc2)OC1(C)C] | 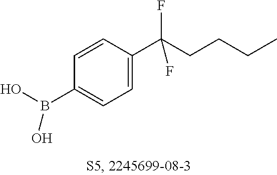 [SMILES: CCCCC(F)(F)c1ccc(B(O)O)cc1] | 49% |
| | 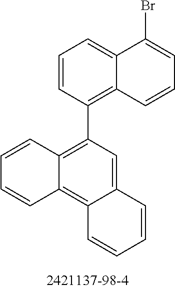 [SMILES: Brc1cccc2c(-c3cc4ccccc4c4ccccc34)cccc12] | | |
| 5 | 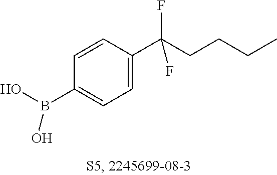 [SMILES: CCCCC(F)(F)c1ccc(B(O)O)cc1] | 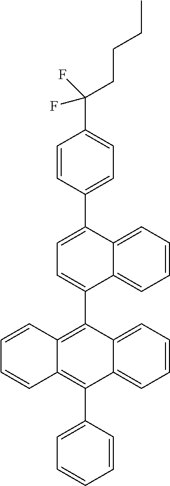 [SMILES: CCCCC(F)(F)c1ccc(-c2ccc(-c3c4ccccc4c(-c4ccccc4)c4ccccc34)c3ccccc23)cc1] | 70% |
| | 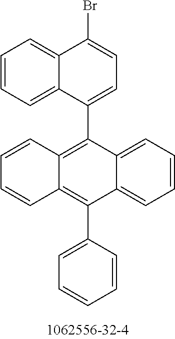 [SMILES: Brc1ccc(-c2c3ccccc3c(-c3ccccc3)c3ccccc23)c2ccccc12] | | |
| 6 | 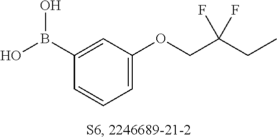 [SMILES: CCC(F)(F)COc1cccc(B(O)O)c1] | 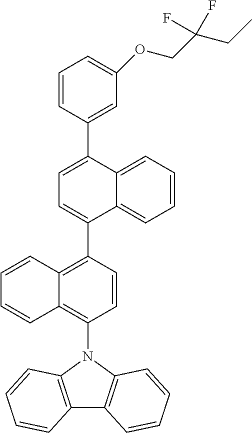 [SMILES: CCC(F)(F)COc1cccc(-c2ccc(-c3ccc(-n4c5ccccc5c5ccccc54)c4ccccc34)c3ccccc23)c1] | 58% |
| | 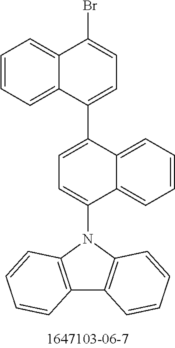 [SMILES: Brc1ccc(-c2ccc(-n3c4ccccc4c4ccccc43)c3ccccc23)c2ccccc12] | | |
| 7 | 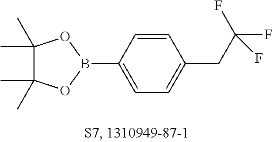 [SMILES: CC1(C)OB(c2ccc(CC(F)(F)F)cc2)OC1(C)C] | 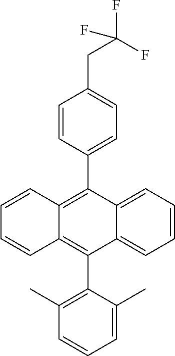 [SMILES: Cc1cccc(C)c1-c1c2ccccc2c(-c2ccc(CC(F)(F)F)cc2)c2ccccc12] | 65% |
| | 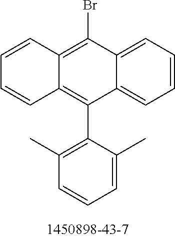 [SMILES: Cc1cccc(C)c1-c1c2ccccc2c(Br)c2ccccc12] | | |
| 8 | 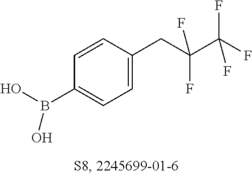 [SMILES: OB(O)c1ccc(CC(F)(F)C(F)(F)F)cc1] | 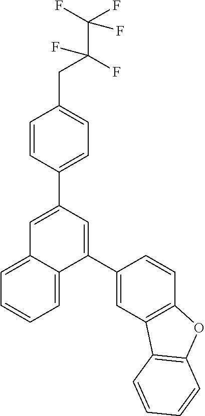 [SMILES: FC(F)(F)C(F)(F)Cc1ccc(-c2cc(-c3ccc4oc5ccccc5c4c3)c3ccccc3c2)cc1] | 66% |
| | 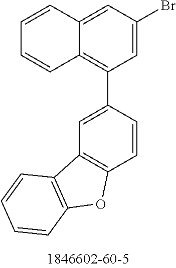 [SMILES: Brc1cc(-c2ccc3oc4ccccc4c3c2)c2ccccc2c1] | | |
| 9 | 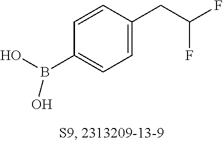 [SMILES: OB(O)c1ccc(CC(F)F)cc1] | 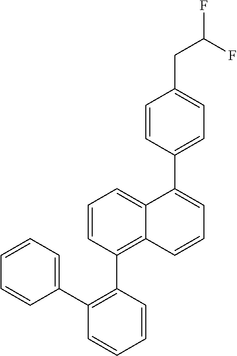 [SMILES: FC(F)Cc1ccc(-c2cccc3c(-c4ccccc4-c4ccccc4)cccc23)cc1] | 71% |
| | 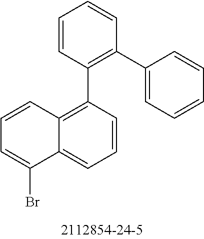 [SMILES: Brc1cccc2c(-c3ccccc3-c3ccccc3)cccc12] | | |
| 10 | 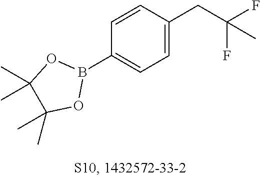 [SMILES: CC(F)(F)Cc1ccc(B2OC(C)(C)C(C)(C)O2)cc1] | 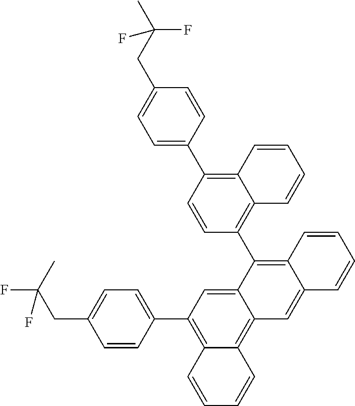 [SMILES: CC(F)(F)Cc1ccc(-c2ccc(-c3c4ccccc4cc4c3cc(-c3ccc(CC(C)(F)F)cc3)c3ccccc34)c3ccccc23)cc1] | 70% |
| | 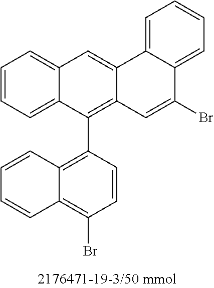 [SMILES: Brc1ccc(-c2c3ccccc3cc3c2cc(Br)c2ccccc23)c2ccccc12] | | |
| 11 | 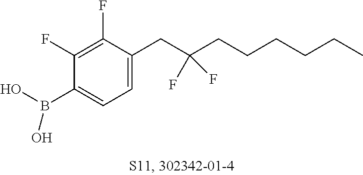 [SMILES: CCCCCCC(F)(F)Cc1ccc(B(O)O)c(F)c1F] | 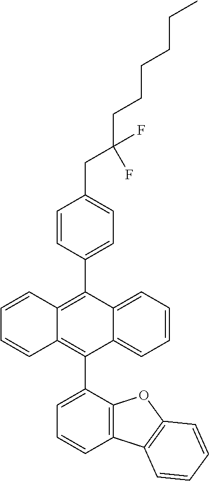 [SMILES: CCCCCCC(F)(F)Cc1ccc(-c2c3ccccc3c(-c3cccc4c3oc3ccccc34)c3ccccc23)cc1] | 61% |
| | 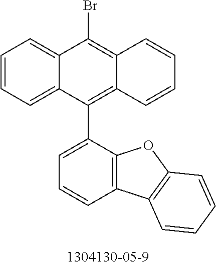 [SMILES: Brc1c2ccccc2c(-c2cccc3c2oc2ccccc23)c2ccccc12] | | |
| 12 | 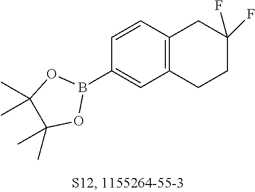 [SMILES: CC1(C)OB(c2ccc3c(c2)CCC(F)(F)C3)OC1(C)C] | 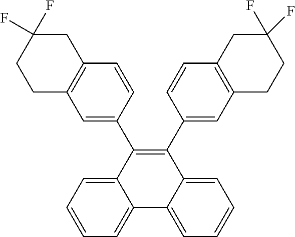 [SMILES: FC1(F)CCc2cc(-c3c(-c4ccc5c(c4)CCC(F)(F)C5)c4ccccc4c4ccccc34)ccc2C1] | 76% |
| | 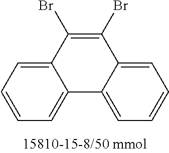 [SMILES: Brc1c(Br)c2ccccc2c2ccccc12] | | |
| 13 | 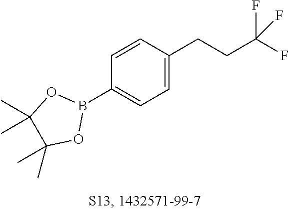 [SMILES: CC1(C)OB(c2ccc(CCC(F)(F)F)cc2)OC1(C)C] | 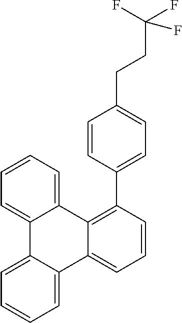 [SMILES: FC(F)(F)CCc1ccc(-c2cccc3c4ccccc4c4ccccc4c23)cc1] | 73% |
| | 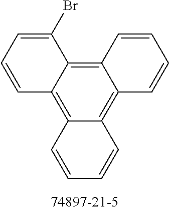 [SMILES: Brc1cccc2c3ccccc3c3ccccc3c12] | | |
| 14 | 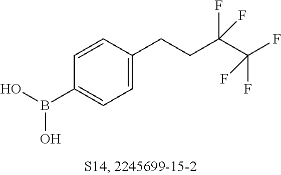 [SMILES: OB(O)c1ccc(CCC(F)(F)C(F)(F)F)cc1] | 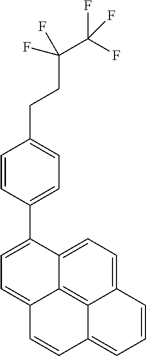 [SMILES: FC(F)(F)C(F)(F)CCc1ccc(-c2ccc3ccc4cccc5ccc2c3c45)cc1] | 69% |
| | 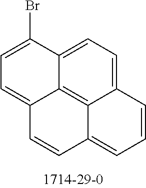 [SMILES: Brc1ccc2ccc3cccc4ccc1c2c34] | | |
| 15 | 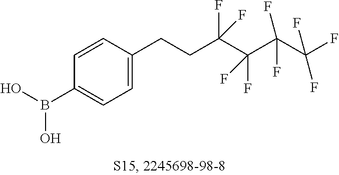 [SMILES: OB(O)c1ccc(CCC(F)(F)C(F)(F)C(F)(F)C(F)(F)F)cc1] | 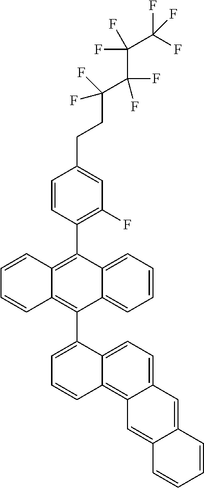 [SMILES: Fc1cc(CCC(F)(F)C(F)(F)C(F)(F)C(F)(F)F)ccc1-c1c2ccccc2c(-c2cccc3c2ccc2cc4ccccc4cc23)c2ccccc12] | 66% |
| | 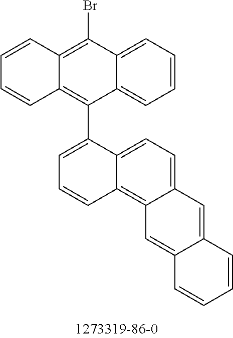 [SMILES: Brc1c2ccccc2c(-c2cccc3c2ccc2cc4ccccc4cc23)c2ccccc12] | | |
| 16 | 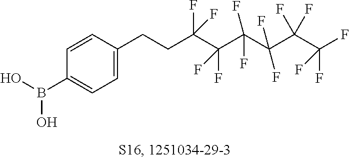 [SMILES: OB(O)c1ccc(CCC(F)(F)C(F)(F)C(F)(F)C(F)(F)C(F)(F)C(F)(F)F)cc1] | 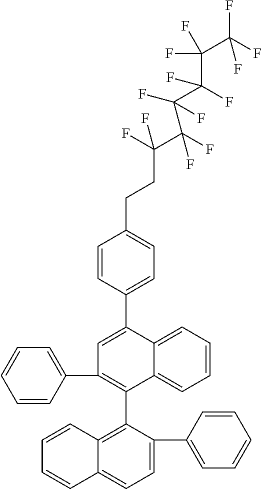 [SMILES: FC(F)(F)C(F)(F)C(F)(F)C(F)(F)C(F)(F)C(F)(F)CCc1ccc(-c2cc(-c3ccccc3)c(-c3c(-c4ccccc4)ccc4ccccc34)c3ccccc23)cc1] | 58% |
| | 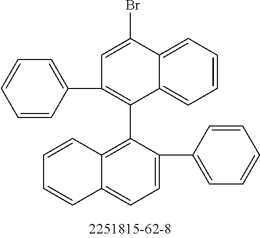 [SMILES: Brc1cc(-c2ccccc2)c(-c2c(-c3ccccc3)ccc3ccccc23)c2ccccc12] | | |
| 17 | 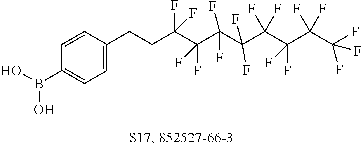 [SMILES: OB(O)c1ccc(CCC(F)(F)C(F)(F)C(F)(F)C(F)(F)C(F)(F)C(F)(F)C(F)(F)C(F)(F)F)cc1] | 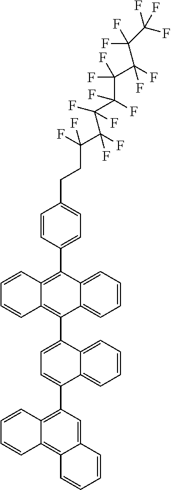 [SMILES: FC(F)(F)C(F)(F)C(F)(F)C(F)(F)C(F)(F)C(F)(F)C(F)(F)C(F)(F)CCc1ccc(-c2c3ccccc3c(-c3ccc(-c4cc5ccccc5c5ccccc45)c4ccccc34)c3ccccc23)cc1] | 54% |
| | 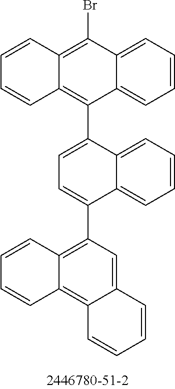 [SMILES: Brc1c2ccccc2c(-c2ccc(-c3cc4ccccc4c4ccccc34)c3ccccc23)c2ccccc12] | | |
| 18 | 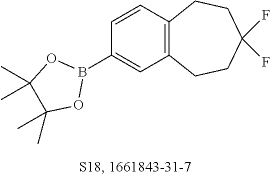 [SMILES: CC1(C)OB(c2ccc3c(c2)CCC(F)(F)CC3)OC1(C)C] | 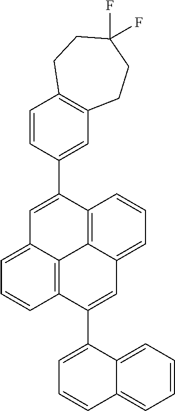 [SMILES: FC1(F)CCc2ccc(-c3cc4cccc5c(-c6cccc7ccccc67)cc6cccc3c6c45)cc2CC1] | 75% |
| | 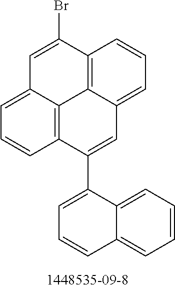 [SMILES: Brc1cc2cccc3c(-c4cccc5ccccc45)cc4cccc1c4c23] | | |
| 19 | 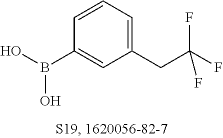 [SMILES: OB(O)c1cccc(CC(F)(F)F)c1] | 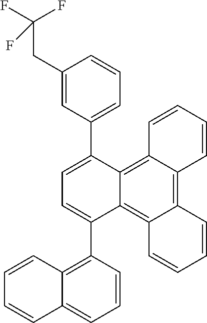 [SMILES: FC(F)(F)Cc1cccc(-c2ccc(-c3cccc4ccccc34)c3c4ccccc4c4ccccc4c23)c1] | 68% |
| | 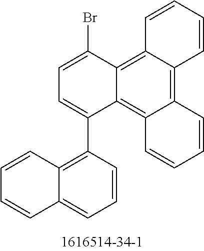 [SMILES: Brc1ccc(-c2cccc3ccccc23)c2c3ccccc3c3ccccc3c12] | | |
| 20 | 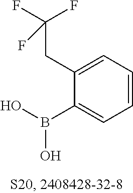 [SMILES: OB(O)c1ccccc1CC(F)(F)F] | 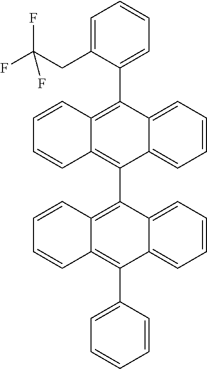 [SMILES: FC(F)(F)Cc1ccccc1-c1c2ccccc2c(-c2c3ccccc3c(-c3ccccc3)c3ccccc23)c2ccccc12] | 63% |
| | 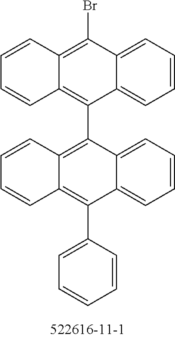 [SMILES: Brc1c2ccccc2c(-c2c3ccccc3c(-c3ccccc3)c3ccccc23)c2ccccc12] | | |
| 21 | 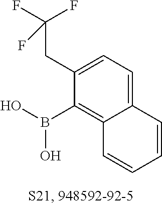 [SMILES: OB(O)c1c(CC(F)(F)F)ccc2ccccc12] | 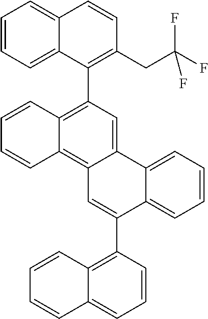 [SMILES: FC(F)(F)Cc1ccc2ccccc2c1-c1cc2c3ccccc3c(-c3cccc4ccccc34)cc2c2ccccc12] | 65% |
| | 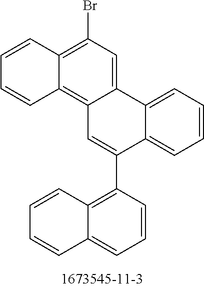 [SMILES: Brc1cc2c3ccccc3c(-c3cccc4ccccc34)cc2c2ccccc12] | | |
| 22 | 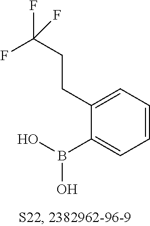 [SMILES: OB(O)c1ccccc1CCC(F)(F)F] | 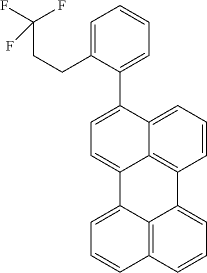 [SMILES: FC(F)(F)CCc1ccccc1-c1ccc2c3cccc4cccc(c5cccc1c52)c43] | 68% |
| | 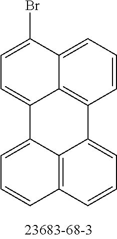 [SMILES: Brc1ccc2c3cccc4cccc(c5cccc1c52)c43] | | |
| 23 | 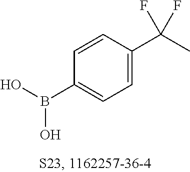 | 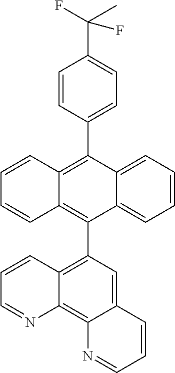 [SMILES: CC(F)(F)c1ccc(-c2c3ccccc3c(-c3cc4cccnc4c4ncccc34)c3ccccc23)cc1] | 63% |
| | 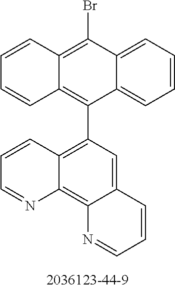 [SMILES: Brc1c2ccccc2c(-c2cc3cccnc3c3ncccc23)c2ccccc12] | | |
| 24 | 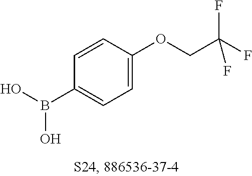 [SMILES: OB(O)c1ccc(OCC(F)(F)F)cc1] | 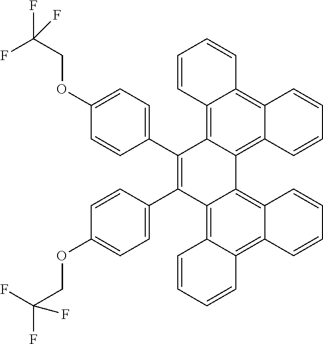 [SMILES: FC(F)(F)COc1ccc(-c2c(-c3ccc(OCC(F)(F)F)cc3)c3c4ccccc4c4ccccc4c3c3c4ccccc4c4ccccc4c23)cc1] | 76% |
| | 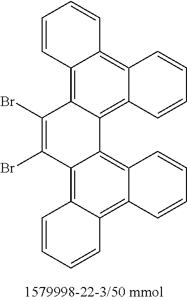 [SMILES: Brc1c(Br)c2c3ccccc3c3ccccc3c2c2c3ccccc3c3ccccc3c12] | | |

### Structuring of Metal Layers:

The examples that follow present the results for structuring of metals and metal mixtures (alloys).

For this purpose, suitable components—as described below—are first produced and then subjected to a measurement of transmittance differential. In the regions in which the compounds of the invention have prevented metal deposition, i.e. structuring has been effected, transmittance is high (>90%).

Cleaned substrates (quartz glass plates, 40×40 mm, cleaning in Miele laboratory glass cleaner, Merck Extran detergent) are pre-treated with UV ozone for 25 minutes (PR-100 UV ozone generator, from UVP). The full area is subjected to vapour deposition under high vacuum (˜10⁻⁵˜10⁻⁷ mbar) of a layer of the electron conductor ETM1, 2-(4-(9,10-di(naphthalen-2-yl)anthracen-2-yl)phenyl)phenyl-1H-benzo[D]imidazole [561064-11-7], thickness 30 nm. Alternatively, it is possible to apply other electron conductors or mixtures of electron conductors that are applied by coevaporation, and other organic functional materials (see table). The layer thicknesses are monitored here by means of a referenced (tooled) crystal oscillator as usual in the prior art in OLED construction.

A shadowmask is used to shadow two quarters, namely region 1=transmittance reference 1 and region 2 of the substrate area. The unshadowed regions 3=transmittance reference 2 and region 4 are each subjected to vapour deposition of layers of the compounds of the invention (for thickness see table). Then regions 1 and 3 are shadowed and 2 and 4 are exposed, and a metal or metal mixture (alloy) is applied by vapour deposition (for deposition rate and thickness see table). The metal mixtures are produced by coevaporation from two separate sources.

The metal deposition is characterized by means of a relative transmittance measurement with light of wavelength 500-550 nm. The transmittance of region 1=transmittance reference 1 serves to correct the transmittance of region 2, i.e. of the glass and the ETM1 layer, and is set to transmittance=100%. The transmittance of region 3=transmittance reference 2 serves to correct the transmittance of region 4, i.e. of the glass, the ETM1 and the layer of the compound of the invention, and is set to transmittance=100%. Then transmittance in regions 2 and 4 is measured and each set relative to references 1 and 3.

In regions in which a metal layer has been deposited, transmittance will be very low or zero; in regions in which very little or no metal layer has been deposited, transmittance will be >90% or more.

TABLE 1

| | Compound of | Transmittance | Transmittance |
|---|---|---|---|
| | the invention | [%] | [%] |
| Ex. | Thickness | Region 2 | Region 4 |
| ETM1 | | | |
| Magnesium, 2 A/s for 1000 s = 200 nm | | | |
| TRef.1 | Ref.1/30 nm | 0 | 98 |
| TRef.2 | Ref.2/30 nm | 0 | 98 |
| TRef.3 | Ref.3/30 nm | 0 | 99 |
| TRef.4 | Ref.1/10 nm | 0 | 45 |
| TRef.5 | Ref.2/10 nm | 0 | 67 |
| TRef.6 | Ref.3/10 nm | 0 | 51 |
| T1 | 1/30 nm | 0 | 97 |
| T2 | 2/30 nm | 0 | 98 |
| T3 | 3/30 nm | 0 | 100 |
| T4 | 1/10 nm | 0 | 90 |
| T5 | 2/10 nm | 0 | 92 |
| T6 | 3/10 nm | 0 | 99 |
| ETM1 | | | |
| Magnesium, 10 A/s for 200 s = 200 nm | | | |
| TRef.1 | Ref.1/30 nm | 0 | 8 |
| TRef.2 | Ref.2/30 nm | 0 | 55 |
| TRef.3 | Ref.3/30 nm | 0 | 38 |
| T100 | 1/30 nm | 0 | 88 |
| T101 | 2/30 nm | 0 | 96 |
| T102 | 3/30 nm | 0 | 99 |
| T103 | 4/30 nm | 0 | 95 |
| T104 | 5/30 nm | 0 | 97 |
| T105 | 6/30 nm | 0 | 98 |
| T106 | 7/30 nm | 0 | 94 |
| T107 | 8/30 nm | 0 | 96 |
| T108 | 9/30 nm | 0 | 95 |
| T109 | 10/30 nm | 0 | 95 |
| T110 | 11/30 nm | 0 | 99 |
| T111 | 12/30 nm | 0 | 96 |
| T112 | 13/30 nm | 0 | 95 |
| T113 | 14/30 nm | 0 | 94 |
| T114 | 15/20 nm | 0 | 97 |
| T115 | 16/30 nm | 0 | 100 |
| T116 | 17/30 nm | 0 | 100 |
| T117 | 18/30 nm | 0 | 100 |
| T118 | 19/30 nm | 0 | 96 |
| T119 | 20/20 nm | 0 | 97 |
| T120 | 21/30 nm | 0 | 92 |
| T121 | 22/30 nm | 0 | 94 |
| T122 | 23/30 nm | 0 | 93 |
| T123 | 24/20 nm | 0 | 100 |
| ETM2 | | | |
| Aluminium, 10 A/s for 200 s = 200 nm | | | |
| T200 | 13/30 nm | 0 | 96 |
| T201 | [1616514-34-1]- | 0 | 97 |
| | S6/30 nm | | |
| T202 | [2351281-21-3]- | 0 | 95 |
| | S12/30 nm | | |
| ETM3 | | | |
| Aluminium, 10 A/s for 200 s = 200 nm | | | |
| T300 | [1062556-32-4]- | 0 | 96 |
| | S2/20 nm | | |
| T301 | [400607-04-7]- | 0 | 99 |
| | S7/20 nm | | |
| ETM4 (50% by wt.)/ETM8 (50% by wt.) | | | |
| Aluminium, 10 A/s for 200 s = 200 nm | | | |
| T400 | 3/20 nm | 0 | 99 |
| T401 | [845457-53-6]- | 0 | 97 |
| | S8/20 nm | | |
| T402 | [1182175-15-0]- | 0 | 96 |
| | S12/20 nm | | |
| ETM5 (50% by wt.)/ETM8 (50% by wt.) | | | |
| Magnesium (10% by wt.)/silver (90% | | | |
| by wt.), 2 A/s for 1000 s = 200 nm | | | |
| T500 | [944801-21-2]- | 0 | 90 |
| | S3/10 nm | | |
| T501 | [1273319-86-0]- | 0 | 92 |
| | S13/10 nm | | |
| ETM6 | | | |
| Magnesium, 10 A/s for 200 s = 200 nm | | | |
| T600 | [15810-15-8]- | 0 | 97 |
| | S14/30 nm | | |
| T601 | [1732-26-9]- | 0 | 98 |
| | S16/30 nm | | |
| ETM7 | | | |
| Silver, 10 A/s for 200 s = 200 nm | | | |
| T700 | [1714-29-0]- | 0 | 94 |
| | S19/15 nm | | |
| T701 | [109465-97-6]- | 0 | 91 |
| | S15/10 nm | | |

Compounds [1616514-34-1]-S6, [2351281-21-3]-S12, [1062556-32-4]-S2, [400607-04-7]-S7, [845457-53-6]-S8, [1182175-15-0]-S12, [944801-21-2]-S3, [1273319-86-0]-S13, [15810-15-8]-S14, [1732-26-9]-S16, [1714-29-0]-S19, [109465-97-6]-S15 are obtained in yields of about 50-90% by reacting the compounds having CAS numbers [1616514-34-1], [2351281-21-3], [1062556-32-4], [400607-04-7], [845457-53-6], [1182175-15-0], [944801-21-2], [1273319-86-0], [15810-15-8], [1732-26-9], [1714-29-0], [109465-97-6], etc. with the above-detailed synthons S2, S3, S6, S7, S8, S12, S13, S14, S15, S16, S19 etc., and analogously to the above-detailed method of preparing compounds 1 to 24, where the regiochemistry of the C—C coupling is fixed unambiguously by the position of the aryl bromide and arylboronic acid coupling partners.

If the aryl bromides are di-, tri-, tetrabromides etc., the stoichiometry is adjusted correspondingly such that all Br functions react under C—C coupling.

The expression [1616514-341]-S6″ represents the product that is obtained by reacting compound CAS No. 1616514-341 with the aforementioned boronic ester S6. The same applies to the other products specified in the above table.

TABLE 2
Structural formulae of the materials used

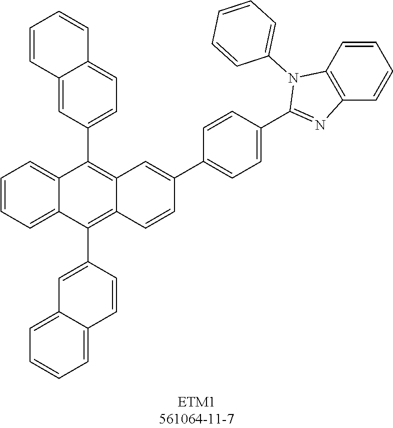 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

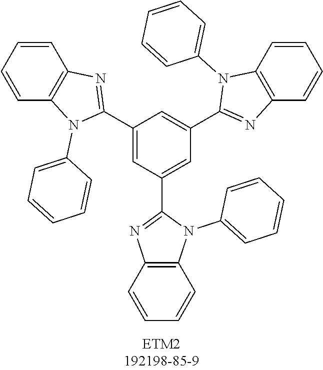 [SMILES: c1ccc(-n2c(-c3cc(-c4nc5ccccc5n4-c4ccccc4)cc(-c4nc5ccccc5n4-c4ccccc4)c3)nc3ccccc32)cc1]

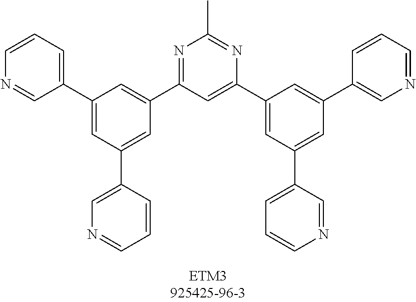 [SMILES: Cc1nc(-c2cc(-c3cccnc3)cc(-c3cccnc3)c2)cc(-c2cc(-c3cccnc3)cc(-c3cccnc3)c2)n1]

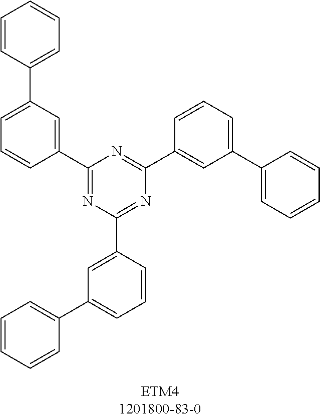 [SMILES: c1ccc(-c2cccc(-c3nc(-c4cccc(-c5ccccc5)c4)nc(-c4cccc(-c5ccccc5)c4)n3)c2)cc1]

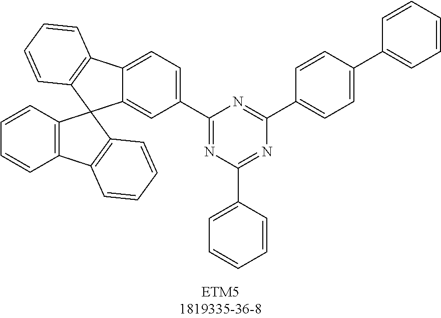 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

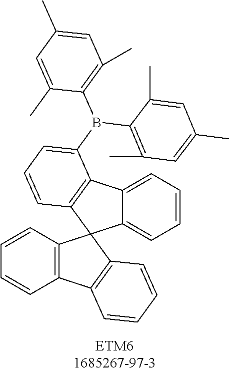 [SMILES: Cc1cc(C)c(B(c2cccc3c2-c2ccccc2C32c3ccccc3-c3ccccc32)c2c(C)cc(C)cc2C)c(C)c1]

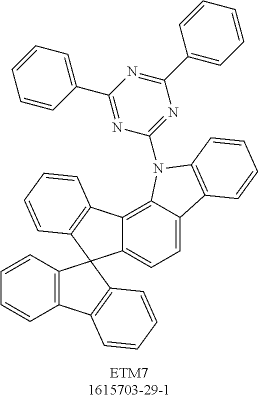 [SMILES: c1ccc(-c2nc(-c3ccccc3)nc(-n3c4ccccc4c4ccc5c(c43)-c3ccccc3C53c4ccccc4-c4ccccc43)n2)cc1]

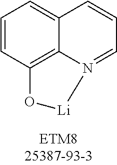

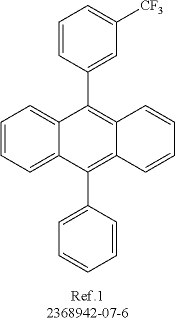 [SMILES: FC(F)(F)c1cccc(-c2c3ccccc3c(-c3ccccc3)c3ccccc23)c1]

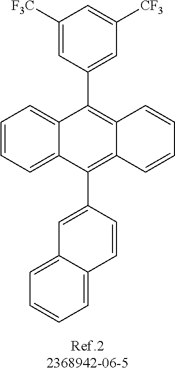 [SMILES: FC(F)(F)c1cc(-c2c3ccccc3c(-c3ccc4ccccc4c3)c3ccccc23)cc(C(F)(F)F)c1]

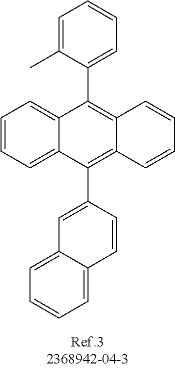 [SMILES: Cc1ccccc1-c1c2ccccc2c(-c2ccc3ccccc3c2)c2ccccc12]

1.-27. (canceled)

28. A method comprising structuring at least one functional layer of an organic electronic device, with a compound, characterized in that the compound comprises at least one fluorinated alkyl radical having at least two carbon atoms.

29. The method according to claim 28, characterized in that the fluorinated alkyl radical comprises at least 2 fluorine atoms.

30. The method according to claim 28, characterized in that the fluorinated alkyl radical has a numerical ratio of fluorine atoms to carbon atoms of at least 0.5.

31. The method according to claim 28, characterized in that the fluorinated alkyl radical has a block structure where some of the carbon atoms have bonds to at least two hydrogen atoms and some of carbon atoms have bonds to at least two fluorine atoms, where the fluorinated alkyl radical has a structure of the formulae FA-1 to FA-16 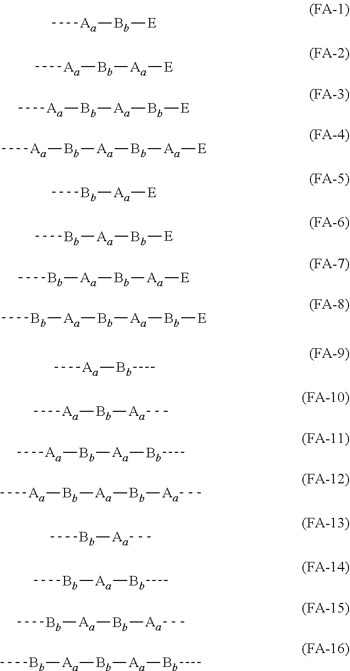
where the dotted line represents the site of attachment of the fluorinated alkyl radical, and in addition:
A is a group of the formula —(CₓH₂ₓ)—, —(CₓHₓDₓ)—, —(CₓD₂ₓ)—, where x is an integer in the range from 1 to 6 where A is;
B is a group of the formula —(C_{y}F_{2y})—, —(C_{y}F_{y}H_{y})—, —(C_{y}F_{y}D_{y})—, where y is an integer in the range from 1 to 6;
E is selected from H, D or F, preferably F;
a is an integer in the range from 1 to 6;
b is an integer in the range from 1 to 6;
where the structures of the formulae (FA-9) to (FA-16) may form a ring, and are connected at two sites to further groups in the compound.

32. The method according to claim 28, characterized in that the compound comprises at least one structuring element of the formula (SE-I), (SE-II) and/or (SE-III) 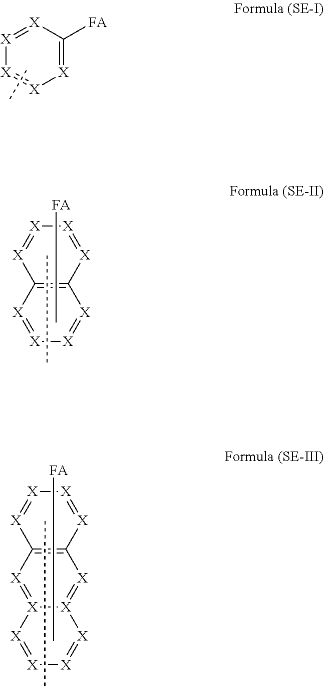
where the FA group is a fluorinated alkyl radical which has at least two carbon atoms and may be substituted by one or more R radicals, the dotted bond represents the bonding site, and in addition:
X is CR, N or C if a group binds to X;
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more R¹ radicals together may form a ring system; at the same time, one or more R¹ radicals may form a ring system with a further part of the compound;
Ar″ is the same or different at each instance and is an aromatic or heteroaromatic ring system which has 5 to 30 aromatic ring atoms and may be substituted by one or more R² radicals; at the same time, it is possible for two Ar″ radicals bonded to the same carbon atom, silicon atom, nitrogen atom, phosphorus atom or boron atom also to be joined together via a bridge by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more substituents R² together may form a ring system.

33. The method according to claim 32, characterized in that the FA group of the structuring element of formula (SE-I), (SE-II) and/or (SE-III) comprises, at least one of the structures of the formulae (FA-1) to (FA-16) 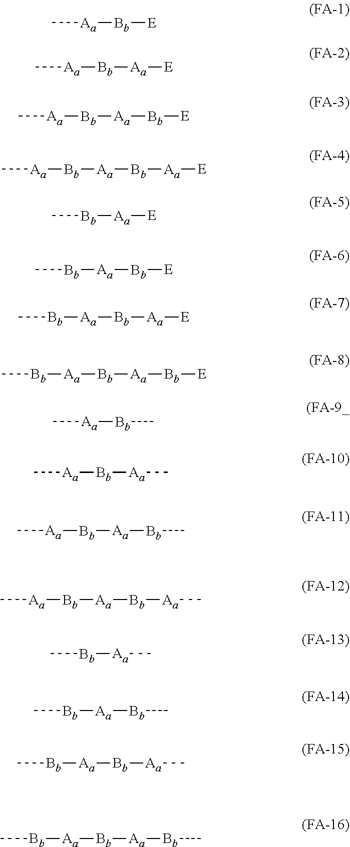
where the dotted line represents the site of attachment of the fluorinated alkyl radical, and in addition:
A is a group of the formula —(CₓH₂ₓ)—, —(CₓHₓDₓ)—, —(CₓD₂ₓ)—, where x is an integer in the range from 1 to 6;
B is a group of the formula —(C_{y}F_{2y})—, —(C_{y}F_{y}H_{y})—, —(C_{y}F_{y}D_{y})—, where y is an integer in the range from 1 to 6;
E is selected from H, D or F, preferably F;
a is an integer in the range from 1 to 6
b is an integer in the range from 1 to 6
where the structures of the formulae (FA-9) to (FA-16) may form a ring, and are connected at two sites to further groups in the compound.

34. The method according to claim 32, characterized in that the structuring element of the formula (SE-I), (SE-II) and/or (SE-III) can be represented by a formula (SE-1) to (SE-21) 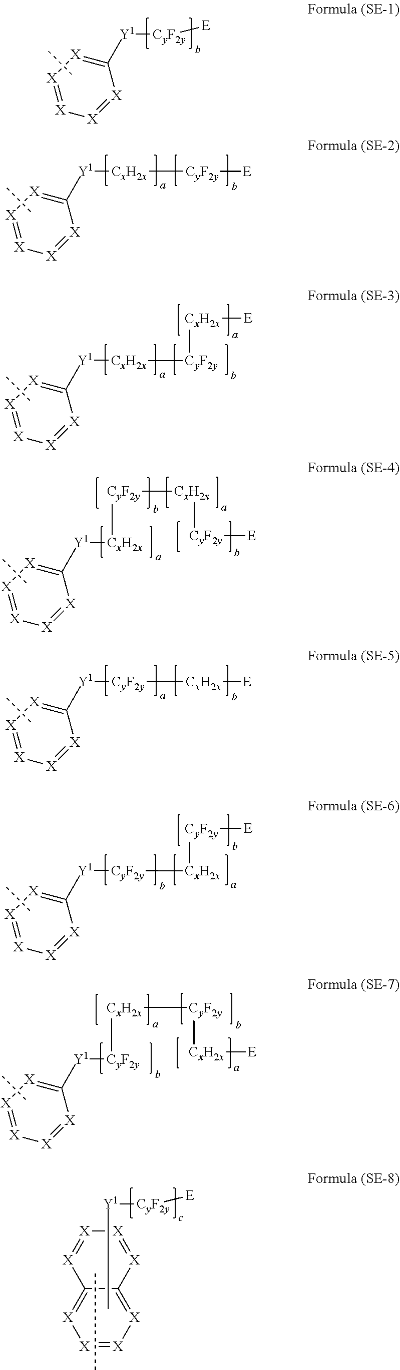 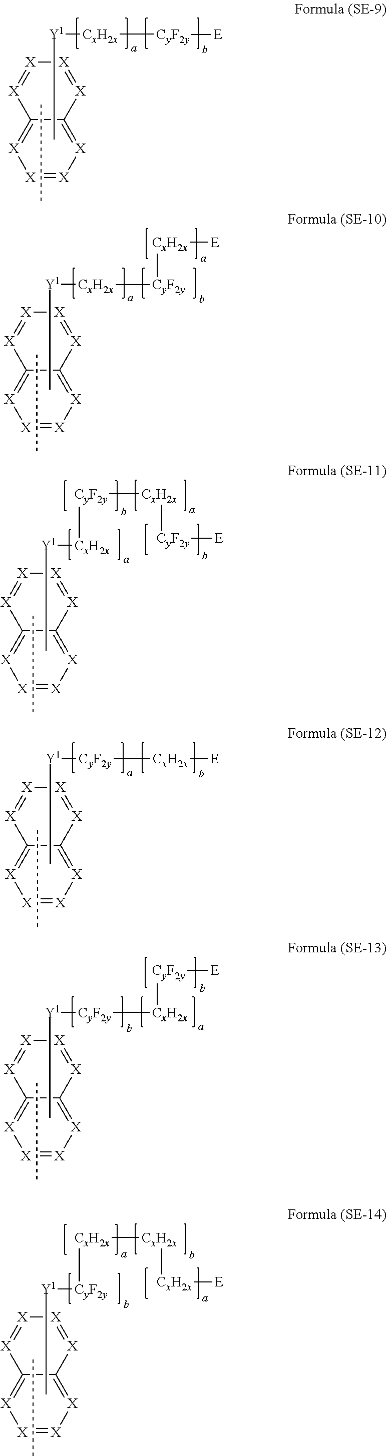 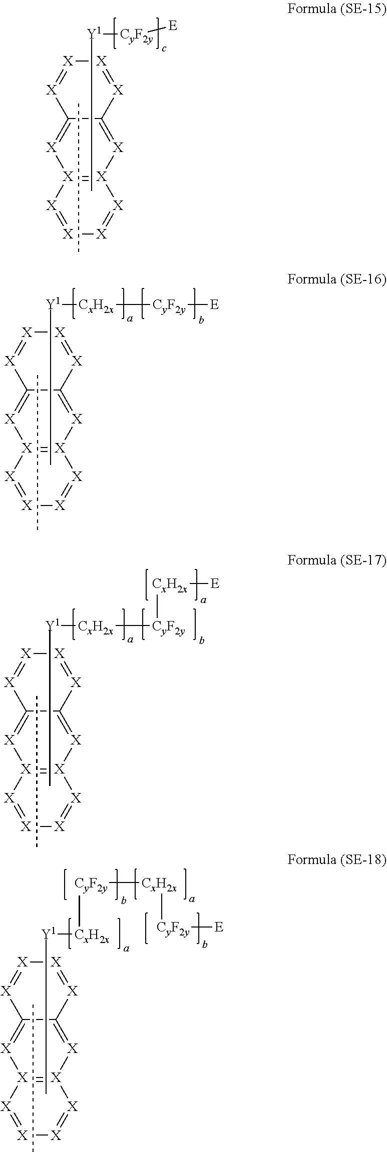 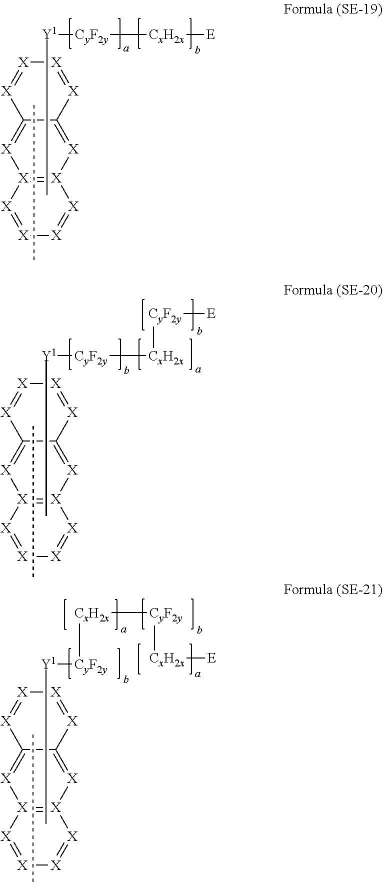
where the symbol X has the definition given in claim 32, and the further symbols are as follows:
E is selected from H, D or F;
Y¹ is the same or different at each instance and is a bond, O, S, NR³ or C(═O);
R³ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, the R³ radical may form a ring system together with an adjacent R or R¹ radical, where the R² and Ar″ radicals have the definition given in claim 32;
the index a is the same or different at each instance and is 1, 2, 3, 4, 5 or 6;
the index b is the same or different at each instance and is 1, 2, 3, 4, 5 or 6;
the index c is 2, 3, 4, 5, 6, 7, 8, 9 or 10;
the index x is the same or different at each instance and is 1, 2, 3, 4, 5 or 6;
the index y is the same or different at each instance and is 1, 2, 3, 4, 5 or 6.

35. The method according to claim 32, characterized in that the structuring element of the formula (SE-I), (SE-II) and/or (SE-III) can be represented by a formula (SE-1a) to (SE-21a) 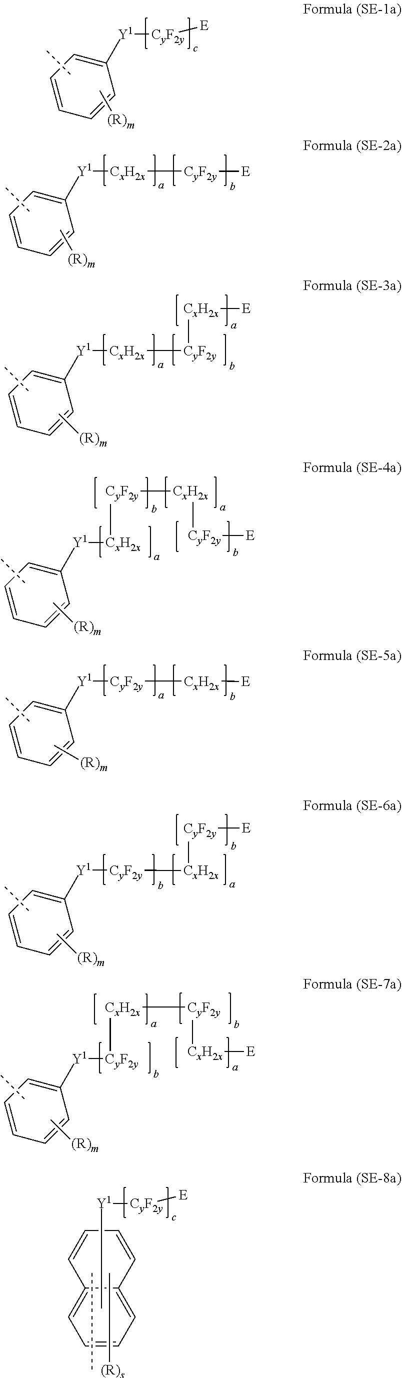 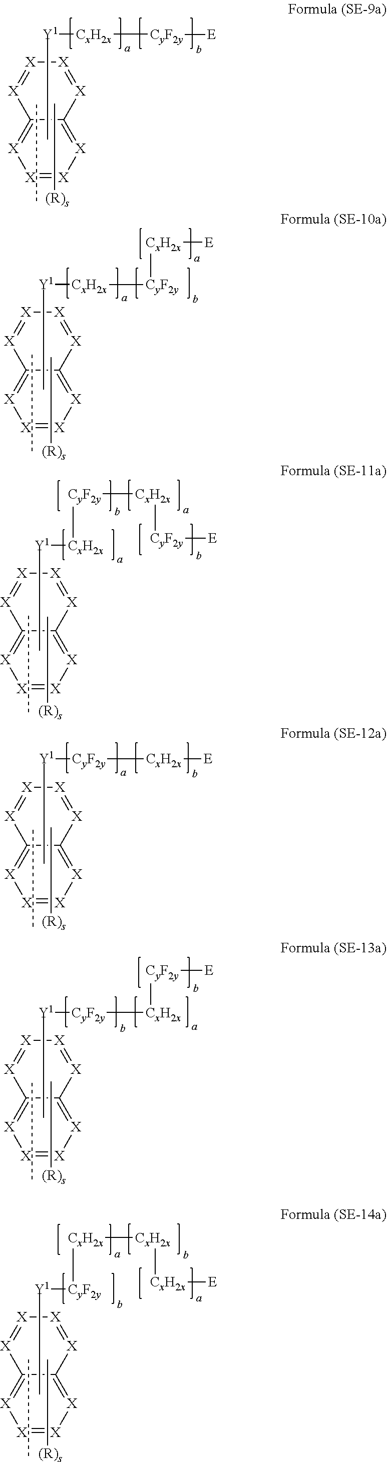 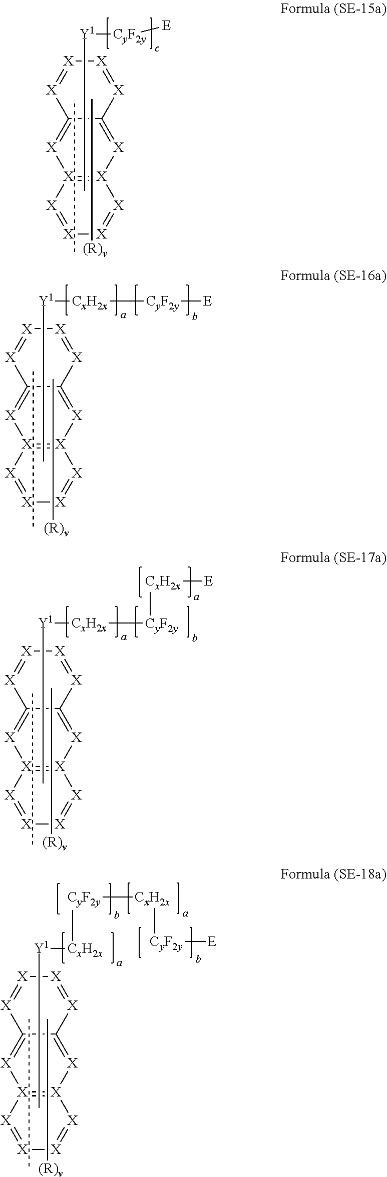 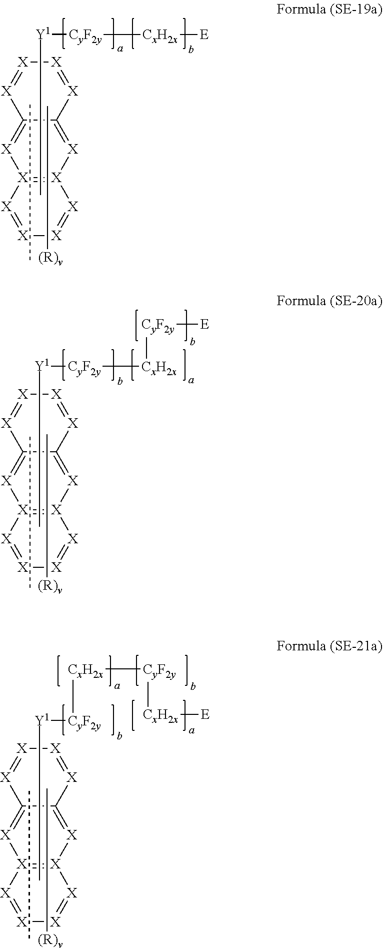
where the symbols are as follows:
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
E is selected from H, D or F;
Y¹ is the same or different at each instance and is a bond, O, S, NR³ or C(═O),
the index a is the same or different at each instance and is 1, 2, 3, 4, 5 or 6;
the index b is the same or different at each instance and is 1, 2, 3, 4, 5 or 6;
the index c is 2, 3, 4, 5, 6, 7, 8, 9 or 10;
the index x is the same or different at each instance and is 1, 2, 3, 4, 5 or 6;
the index y is the same or different at each instance and is 1, 2, 3, 4, 5 or 6;
m is 0, 1, 2, 3 or 4, preferably 0, 1 or 2;
s is 0, 1, 2, 3, 4, 5 or 6, preferably 0, 1, 2, 3 or 4;
v is 0, 1, 2, 3, 4, 5, 6, 7 or 8, preferably 0, 1, 2, 3 or 4.

36. The method according to claim 28, characterized in that the compound comprises at least one aromatic or heteroaromatic ring system having at least two fused aromatic or heteroaromatic rings.

37. The method according to claim 36, characterized in that the aromatic or heteroaromatic ring system having two, fused aromatic or heteroaromatic rings is selected from the groups of the formulae (Ar′-1) to (Ar′-18) 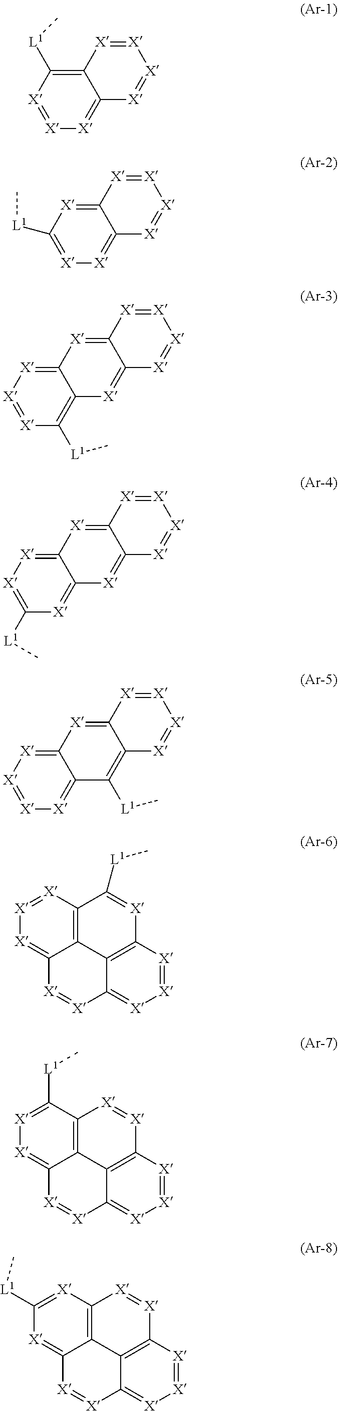 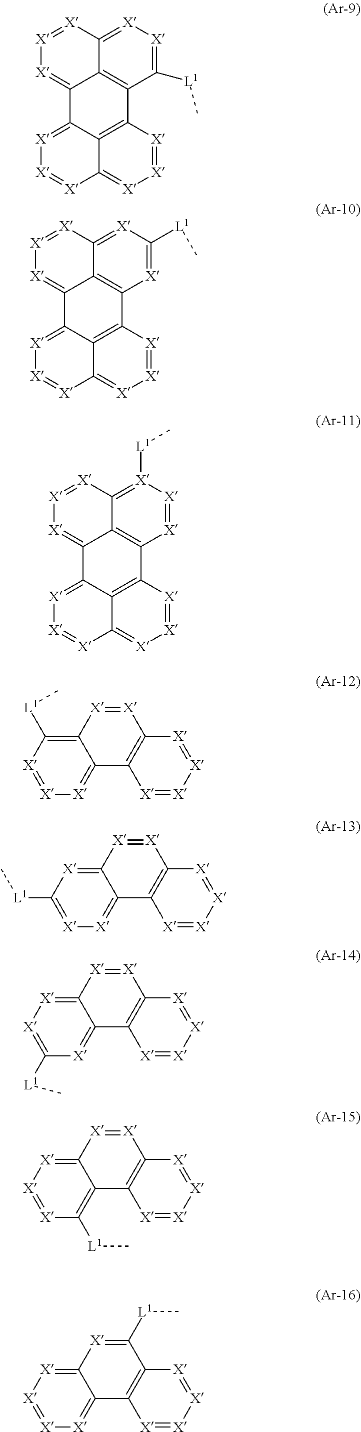 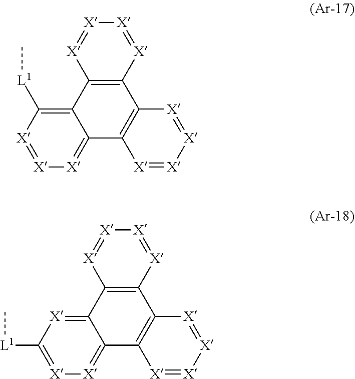
where X′ is N or CR^{a}, L¹ represents a bond or an aromatic or heteroaromatic ring system which has 5 to 40, aromatic ring atoms and may be substituted by one or more R¹ radicals, where the dotted bond marks the position of attachment, and in addition:
R^{a} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R^{a} radicals may also form a ring system with one another or a further group, for example with one or more of the R or R¹ radicals, where the symbols R¹ and Ar′ are defined as follows:
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound.

38. A compound comprising at least one structure of the formula (I), suitable for the method according to claim 28, 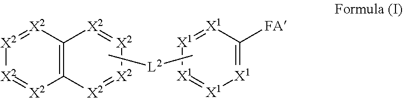
where the FA′ group is a fluorinated alkyl radical which has at least two carbon atoms and may be substituted by one or more R radicals, and the symbols are as follows:
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R′, P(═O)(Ar′)₂, P(═O)(R¹)₂, P(Ar′)₂, P(R¹)₂, S(═O)Ar′, S(═O)R′, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
X¹ is CR^{b} if the L² group binds to X¹, or N or C;
X² is CR^{c} if the L² group binds to X², or N or C;
L² is a linking group, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
R^{b} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R′, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R^{b} radicals may also form a ring system with one another or a further group, for example with one or more of the R^{c} radicals, where the symbols R¹ and Ar′ are defined as follows:
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O) NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
R^{c} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R^{c} radicals may also form a ring system with one another or a further group, for example with one or more of the R^{b} radicals, where the symbols R¹ and Ar′ are defined as follows:
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound.

39. A compound comprising at least one structure of the formula (I-1) to (I-7), suitable for the method according to claim 28, 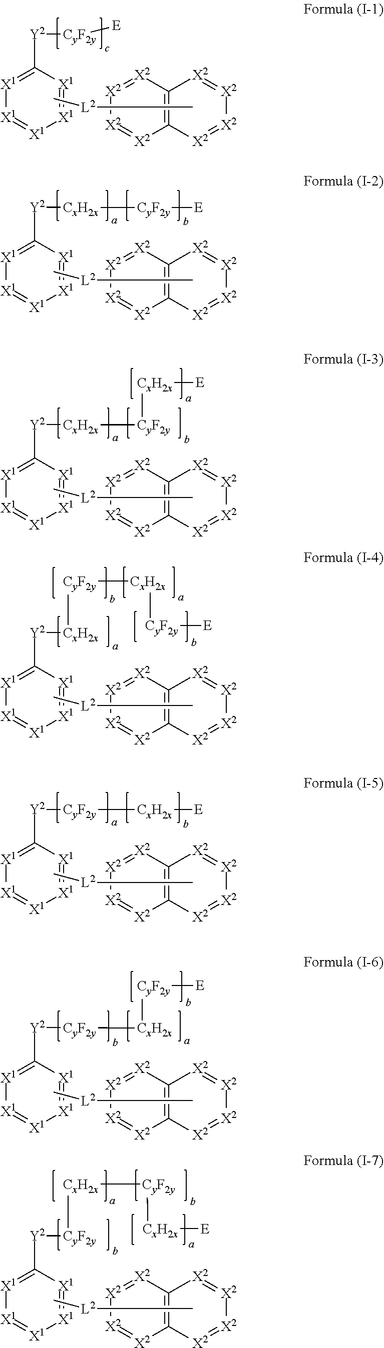
where the symbols are as follows:
X¹ is CR^{b} if the L² group binds to X¹, or N or C;
X² is CR^{c} if the L² group binds to X², or N or C;
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
E is selected from H, D or F;
Y² is the same or different at each instance and is a bond, O, S, NR⁴ or C(═O);
R⁴ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, the R⁴ radical may form a ring system with a further part of the compound, where the symbol R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, adjacent substituents R² together may form a ring system;
a is the same or different at each instance and is 1, 2, 3, 4, 5 or 6;
b is the same or different at each instance and is 1, 2, 3, 4, 5 or 6;
c is 2, 3, 4, 5, 6, 7, 8, 9 or 10, preferably 2, 3, 4, 5 or 6;
x is the same or different at each instance and is 1, 2, 3, 4, 5 or 6;
y is the same or different at each instance and is 1, 2, 3, 4, 5 or 6.

40. A compound comprising at least one structure of the formula (II), preferably a compound of formula (II), suitable for the method according to claim 28, 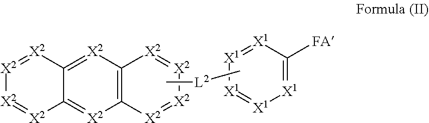
where the FA′ group is a fluorinated alkyl radical which has at least two carbon atoms and may be substituted by one or more R radicals, where the symbols are as follows:
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, —C≡C—, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
X¹ is CR^{b} if the L² group binds to X¹, or N or C;
X² is CR^{c} if the L² group binds to X², or N or C;
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound.

41. A compound comprising at least one structure of the formulae (II-1) to (II-7), suitable for the method according to claim 28, 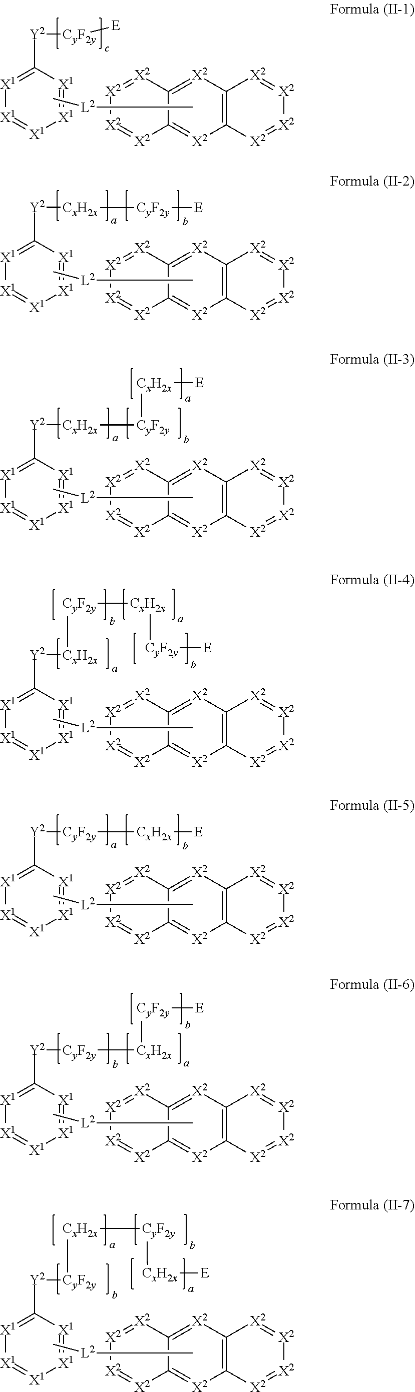
where the symbols are as follows:
X¹ is CR^{b} if the L² group binds to X¹, or N or C;
X² is CR^{c} if the L² group binds to X², or N or C;
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
E is selected from H, D or F;
Y² is the same or different at each instance and is a bond, O, S, NR⁴ or C(═O);
a is the same or different at each instance and is 1, 2, 3, 4, 5 or 6;
b is the same or different at each instance and is 1, 2, 3, 4, 5 or 6;
c is 2, 3, 4, 5, 6, 7, 8, 9 or 10, preferably 2, 3, 4, 5 or 6;
x is the same or different at each instance and is 1, 2, 3, 4, 5 or 6;
y is the same or different at each instance and is 1, 2, 3, 4, 5 or 6.

42. A compound comprising at least one structure of the formula (III), suitable for the method according to claim 28, 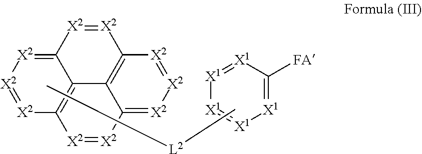
where the FA′ group is a fluorinated alkyl radical which has at least two carbon atoms and may be substituted by one or more R radicals, where the symbols are as follows:
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
X¹ is CR^{b} if the L² group binds to X¹, or N or C;
X² is CR^{c} if the L² group binds to X², or N or C.

43. A compound comprising at least one structure of the formulae (III-1) to (III-7), suitable for the method according to claim 28, 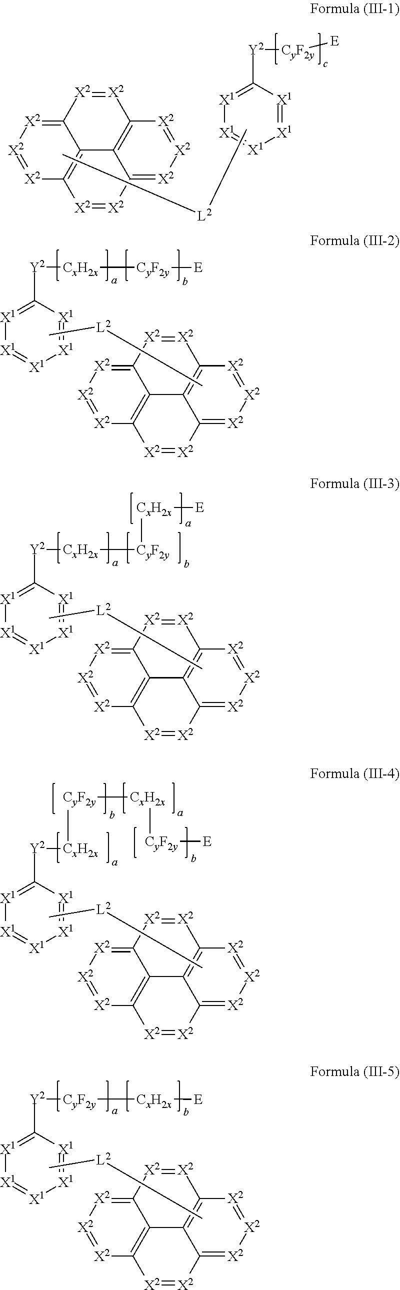 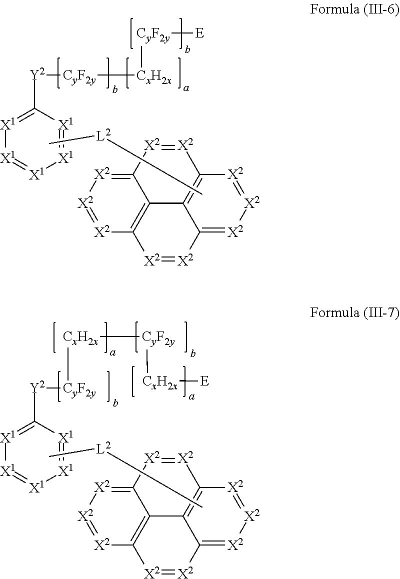
where the symbols are as follows:
L² is a linking group, a bond or an aromatic or heteroaromatic ring system which has 5 to 40 aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
X¹ is CR^{b} if the L² group binds to X¹, or N or C;
X² is CR^{c} if the L² group binds to X², or N or C;
E is selected from H, D or F;
Y² is the same or different at each instance and is a bond, O, S, NR⁴ or C(═O);
a is the same or different at each instance and is 1, 2, 3, 4, 5 or 6;
b is the same or different at each instance and is 1, 2, 3, 4, 5 or 6;
c is 2, 3, 4, 5, 6, 7, 8, 9 or 10, preferably 2, 3, 4, 5 or 6;
x is the same or different at each instance and is 1, 2, 3, 4, 5 or 6;
y is the same or different at each instance and is 1, 2, 3, 4, 5 or 6.

44. A compound comprising at least one structure of the formula (IV), suitable for the method according to claim 28, 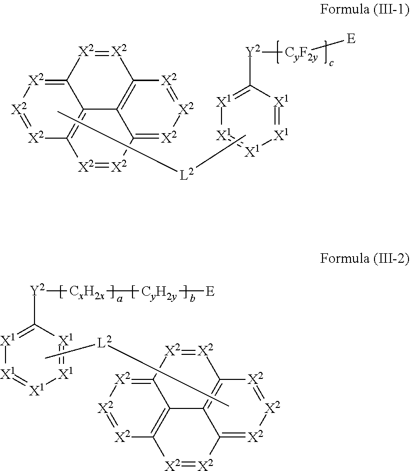
where the FA′ group is a fluorinated alkyl radical which has at least two carbon atoms and may be substituted by one or more R radicals, where the symbols are as follows:
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
X¹ is CR^{b} if the L² group binds to X¹, or N or C;
X² is CR^{c} if the L² group binds to X², or N or C.

45. A compound comprising at least one structure of the formulae (IV-1) to (IV-7), suitable for the method according to claim 28, 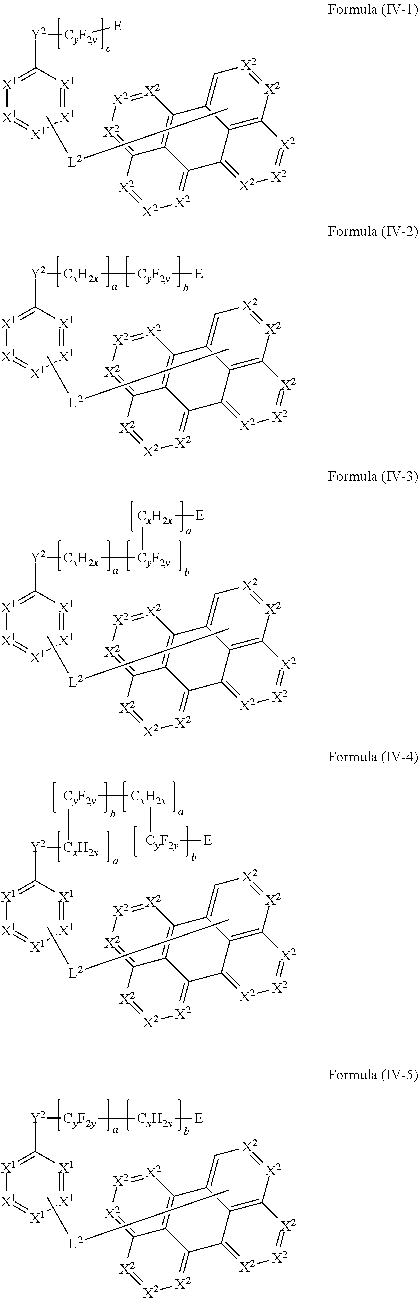 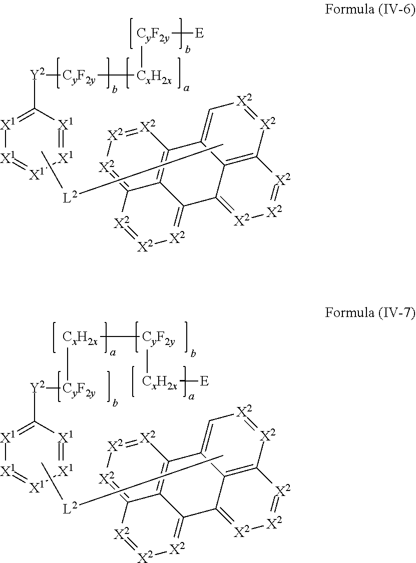
where the symbols are as follows:
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
X¹ is CR^{b} if the L² group binds to X¹, or N or C;
X² is CR^{c} if the L² group binds to X², or N or C;
E is selected from H, D or F, H or F;
Y² is the same or different at each instance and is a bond, O, S, NR⁴ or C(═O);
a is the same or different at each instance and is 1, 2, 3, 4, 5 or 6;
b is the same or different at each instance and is 1, 2, 3, 4, 5 or 6;
c is 2, 3, 4, 5, 6, 7, 8, 9 or 10, preferably 2, 3, 4, 5 or 6;
x is the same or different at each instance and is 1, 2, 3, 4, 5 or 6;
y is the same or different at each instance and is 1, 2, 3, 4, 5 or 6.

46. A compound comprising at least one structure of the formula (V), preferably a compound of formula (V), suitable for use according to claim 28, 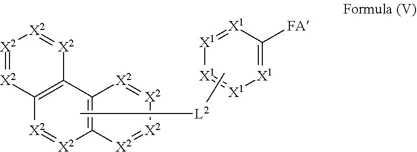
where the FA′ group is a fluorinated alkyl radical which has at least two carbon atoms and may be substituted by one or more R radicals, but is preferably unsubstituted, where the symbols are as follows:
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
X¹ is CR^{b} if the L² group binds to X¹, or N or C;
X² is CR^{c} if the L² group binds to X², or N or C.

47. A compound comprising at least one structure of the formulae (V-1) to (V-7), suitable for the method according to claim 28, 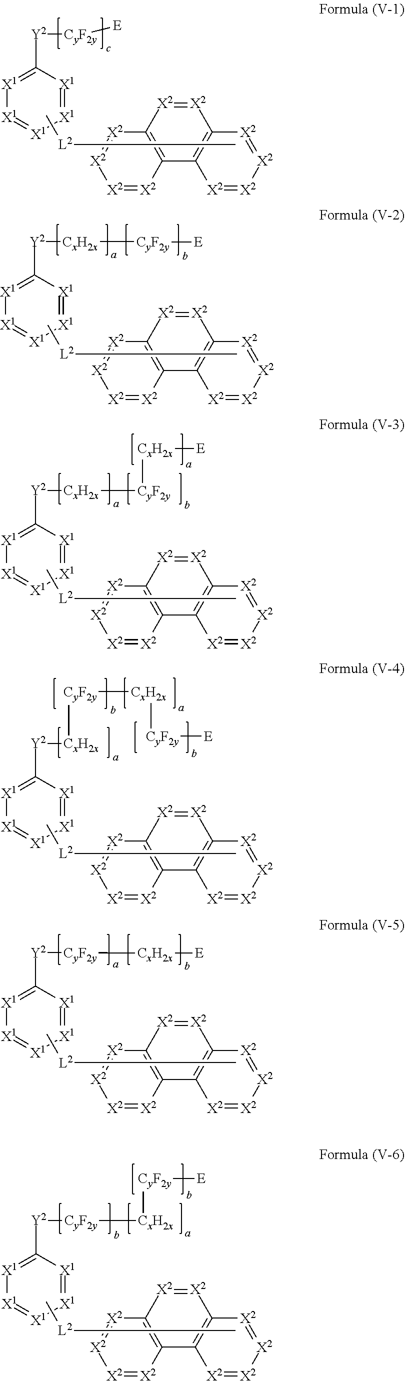 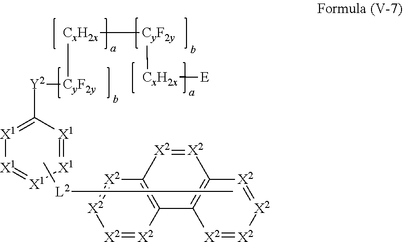
where the symbols are as follows:
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²)², —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
X¹ is CR^{b} if the L² group binds to X¹, or N or C;
X² is CR^{c} if the L² group binds to X², or N or C;
E is selected from H, D or F;
Y² is the same or different at each instance and is a bond, O, S, NR⁴ or C(═O);
a is the same or different at each instance and is 1, 2, 3, 4, 5 or 6;
b is the same or different at each instance and is 1, 2, 3, 4, 5 or 6;
c is 2, 3, 4, 5, 6, 7, 8, 9 or 10;
x is the same or different at each instance and is 1, 2, 3, 4, 5 or 6;
y is the same or different at each instance and is 1, 2, 3, 4, 5 or 6.

48. A compound comprising at least one structure of the formula (VI), suitable for use according to claim 28, 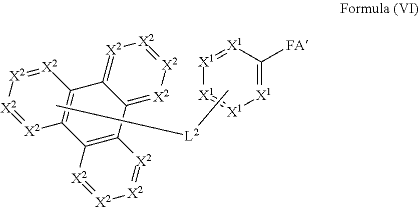
where the FA′ group is a fluorinated alkyl radical which has at least two carbon atoms and may be substituted by one or more R radicals, where the symbols are as follows:
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system with one another or a further group;
L² is a linking group, a bond or an aromatic or heteroaromatic ring system which has 5 to 40, aromatic ring atoms and may be substituted by one or more R¹ radicals, where the symbol R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more adjacent R¹ radicals together may form a ring system; at the same time, one or more R¹ radicals may form a ring system with a further part of the compound;
X¹ is CR^{b} if the L² group binds to X¹, or N or C;
X² is CR^{c} if the L² group binds to X², or N or C.
